SEQUENCE LISTING (126 sequences)

SEQ ID NO: 1 - DNA, 705 bp, synthetic construct
  misc_feature 1..705: note = Description of sequence: light chain protein nucleotide sequence; Clone: 2-3B-G8
  source 1..705: mol_type = other DNA
atggattttcaagtgcagattttcagcttcctgctaatcagtgcctcagtcataatgtccagaggagaaaatgttctcacccagtctccagcaatcatgtctgcatctccaggggaaaaggtcaccatgacctgcagtgccagctcaagtgtaagttacatgcactggtaccagcagaagtcaagcacctcccccaaactctggatttatgacacatccaaactggcttctggagtccctgctcgcttcagtggcagtgggtctgggacctcttactctctcacaatcagcagcatggaggctgaagatgttgccacttattactgttttcaggggagtgggtacccactcacgttcggctcggggacaaagttggaaataaaacgggctgatgctgcaccaactgtatacatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagactgcacctacagcatgagcagcaccctcacgttgaccaaggacgagtatgaacgccataacagctatacctgtgaggccactcacaagacatcaacttcacccattgtcaagagcttcaacaggaatgagtgt

SEQ ID NO: 2 - protein, 235 aa, synthetic construct
  REGION 1..235: note = Description of sequence: light chain protein amino acid sequence; Clone: 2-3B-G8
MDFQVQIFSFLLISASVIMSRGENVLTQSPAIMSASPGEKVTMTCSASSSVSYMHWYQQKSSTSPKLWIYDTSKLASGVPARFSGSGSGTSYSLTISSMEAEDVATYYCFQGSGYPLTFGSGTKLEIKRADAAPTVYIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

SEQ ID NO: 3 - DNA, 384 bp, synthetic construct
  misc_feature 1..384: note = Description of sequence: light chain variable domain nucleotide sequence; Clone: 2-3B-G8
  source 1..384: mol_type = other DNA
atggattttcaagtgcagattttcagcttcctgctaatcagtgcctcagtcataatgtccagaggagaaaatgttctcacccagtctccagcaatcatgtctgcatctccaggggaaaaggtcaccatgacctgcagtgccagctcaagtgtaagttacatgcactggtaccagcagaagtcaagcacctcccccaaactctggatttatgacacatccaaactggcttctggagtccctgctcgcttcagtggcagtgggtctgggacctcttactctctcacaatcagcagcatggaggctgaagatgttgccacttattactgttttcaggggagtgggtacccactcacgttcggctcggggacaaagttggaaataaaa

SEQ ID NO: 4 - protein, 128 aa, synthetic construct
  REGION 1..128: note = Description of sequence: light chain variable domain amino acid sequence; Clone: 2-3B-G8
MDFQVQIFSFLLISASVIMSRGENVLTQSPAIMSASPGEKVTMTCSASSSVSYMHWYQQKSSTSPKLWIYDTSKLASGVPARFSGSGSGTSYSLTISSMEAEDVATYYCFQGSGYPLTFGSGTKLEIK

SEQ ID NO: 5 - DNA, 1212 bp, synthetic construct
  misc_feature 1..1212: note = Description of sequence: heavy chain protein nucleotide sequence; Clone: 2-3B-G8
  source 1..1212: mol_type = other DNA
atggaactctggggacctggcagtaccgatgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaactggacgagacctgtgctgaggcccaggacggggagctggacgggctctggacgaccatcaccatcttcatcagcctcttcctgctcagcgtgtgctacagcgctgctgtcacactcttcaaggtaaagtggatcttctcctcggtggtggagctgaagcagacactggttcctgaatacaagaacatgattgggcaagcgccc

SEQ ID NO: 6 - protein, 404 aa, synthetic construct
  REGION 1..404: note = Description of sequence: heavy chain protein amino acid sequence; Clone: 2-3B-G8
MELWGPGSTDAAKTTAPSVYPLAPVCGDTTGSSVTLGCLVKGYFPEPVTLTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQLDETCAEAQDGELDGLWTTITIFISLFLLSVCYSAAVTLFKVKWIFSSVVELKQTLVPEYKNMIGQAP

SEQ ID NO: 7 - DNA, 33 bp, synthetic construct
  misc_feature 1..33: note = Description of sequence: heavy chain variable domain nucleotide sequence; Clone: 2-3B-G8
  source 1..33: mol_type = other DNA
atggaactctggggacctggcagtaccgatgca

SEQ ID NO: 8 - protein, 11 aa, synthetic construct
  REGION 1..11: note = Description of sequence: heavy chain variable domain amino acid sequence; Clone: 2-3B-G8
MELWGPGSTDA

SEQ ID NO: 9 - protein, 8 aa, synthetic construct
  REGION 1..8: note = Description of sequence: CDRL1 amino acid sequence; Clone: 2-3B-G8
SASSSVSY

SEQ ID NO: 10 - protein, 7 aa, synthetic construct
  REGION 1..7: note = Description of sequence: CDRL2 amino acid sequence; Clone: 2-3B-G8
DTSKLAS

SEQ ID NO: 11 - protein, 9 aa, synthetic construct
  REGION 1..9: note = Description of sequence: CDRL3 amino acid sequence; Clone: 2-3B-G8
FQGSGYPLT

SEQ ID NO: 12 - protein, 235 aa, synthetic construct
  REGION 1..235: note = Description of sequence: light chain protein humanized amino acid sequence; Clone: 2-3B-G8
MDFQVQIFSFLLISASVIMSRGENVLTQSPAIMSASPGEKVTMTCSASSSVSYMHWYQQKSSTSPKLWIYDTSKLASGVPARFSGSGSGTSYSLTISSMEAEDVATYYCFQGSGYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 341 aa, synthetic construct
  REGION 1..341: note = Description of sequence: heavy chain protein humanized amino acid sequence; Clone: 2-3B-G8
MELWGPGSTDAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, synthetic construct
  REGION 1..214: note = Description of sequence: light chain protein humanized amino acid sequence; Clone: 2-3B-G8
MQVVLTQSPGIMSASPGEKVTITCSASSSVSYMYWFQQKPGTSPKLWIYDTSKLASGVPARFRGSGSGTSYSLTISRMEAEDAATYYCFQGSGYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 107 aa, synthetic construct
  REGION 1..107: note = Description of sequence: light chain variable domain humanized amino acid sequence; Clone: 2-3B-G8
MQVVLTQSPGIMSASPGEKVTITCSASSSVSYMYWFQQKPGTSPKLWIYDTSKLASGVPARFRGSGSGTSYSLTISRMEAEDAATYYCFQGSGYPLTFGGGTKLEIK

SEQ ID NO: 16 - protein, 8 aa, synthetic construct
  REGION 1..8: note = Description of sequence: CDRL1 humanized amino acid sequence; Clone: 2-3B-G8
SASSSVSY

SEQ ID NO: 17 - protein, 7 aa, synthetic construct
  REGION 1..7: note = Description of sequence: CDRL2 humanized amino acid sequence; Clone: 2-3B-G8
DTSKLAS

SEQ ID NO: 18 - protein, 9 aa, synthetic construct
  REGION 1..9: note = Description of sequence: CDRL3 humanized amino acid sequence; Clone: 2-3B-G8
FQGSGYPLT

SEQ ID NO: 19 - DNA, 705 bp, synthetic construct
  misc_feature 1..705: note = Description of sequence: light chain protein nucleotide sequence; Clone: 1-4B-B10
  source 1..705: mol_type = other DNA
atggattttcaagtgcagattttcagcttcctgctaatcagtgcctcagtcataatgtccagaggagaaaatgttctcacccagtctccagcaatcatgtctgcatctccaggggaaaaggtcaccatgacctgcagtgccagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgatgaccaaggacgagtatgaacgacataacagctatacctgtgaggccactcacaagacatcaacttcacccattgtcaagagcttcaacaggaatgagtgt

SEQ ID NO: 20 - protein, 235 aa, synthetic construct
  REGION 1..235: note = Description of sequence: light chain protein amino acid sequence; Clone: 1-4B-B10
MDFQVQIFSFLLISASVIMSRGENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIKRADAAPTVSIFPPSSEQLTSGGASVVCFLNNFYPKDINVKWKIDGSERQNGVLNSWTDQDSKDSTYSMSSTLTMTKDEYERHNSYTCEATHKTSTSPIVKSFNRNEC

SEQ ID NO: 21 - DNA, 384 bp, synthetic construct
  misc_feature 1..384: note = Description of sequence: light chain variable domain nucleotide sequence; Clone: 1-4B-B10
  source 1..384: mol_type = other DNA
atggattttcaagtgcagattttcagcttcctgctaatcagtgcctcagtcataatgtccagaggagaaaatgttctcacccagtctccagcaatcatgtctgcatctccaggggaaaaggtcaccatgacctgcagtgccagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaa

SEQ ID NO: 22 - protein, 128 aa, synthetic construct
  REGION 1..128: note = Description of sequence: light chain variable domain amino acid sequence; Clone: 1-4B-B10
MDFQVQIFSFLLISASVIMSRGENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIK

SEQ ID NO: 23 - DNA, 1239 bp, synthetic construct
  misc_feature 1..1239: note = Description of sequence: heavy chain protein nucleotide sequence; Clone: 1-4B-B10
  source 1..1239: mol_type = other DNA
atgtgccgacttcaatttgtgcttcagtgttgtccagattgtgtgcagccatatggcccagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaactggacgagacctgtgctgaggcccaggacggggagctggacgggctctggacgaccatcaccatcttcatcagcctcttcctgctcagcgtgtgctacagcgctgctgtcacactcttcaaggtaaagtggatcttctcctcggtggtggagctgaagcagacactggttcctgaatacaagaacatgattgggcaagcgccc

SEQ ID NO: 24 - protein, 413 aa, synthetic construct
  REGION 1..413: note = Description of sequence: heavy chain protein amino acid sequence; Clone: 1-4B-B10
MCRLQFVLQCCPDCVQPYGPAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQLDETCAEAQDGELDGLWTTITIFISLFLLSVCYSAAVTLFKVKWIFSSVVELKQTLVPEYKNMIGQAP

SEQ ID NO: 25 - protein, 5 aa, synthetic construct
  REGION 1..5: note = Description of sequence: CDRL1 amino acid sequence; Clone: 1-4B-B10
SSVSY

SEQ ID NO: 26 - skipped

SEQ ID NO: 27 - protein, 9 aa, synthetic construct
  REGION 1..9: note = Description of sequence: CDRL3 amino acid sequence; Clone: 1-4B-B10
FQGSGYPLT

SEQ ID NO: 28 - protein, 235 aa, synthetic construct
  REGION 1..235: note = Description of sequence: light chain protein humanized amino acid sequence; Clone: 1-4B-B10
MDFQVQIFSFLLISASVIMSRGENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 756 bp, synthetic construct
  misc_feature 1..756: note = Description of sequence: light chain protein nucleotide sequence; Clone: 1-3C-F11
  source 1..756: mol_type = other DNA
atgagaagagaagtaattagccagggaacaaaattcaaatacacaatggattttctggtgcagattttcagcttcttgctaatcagtgcctcagttgcaatgtccagaggagaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttacccactaacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 30 - protein, 252 aa, synthetic construct
  REGION 1..252: note = Description of sequence: light chain protein amino acid sequence; Clone: 1-3C-F11
MRREVISQGTKFKYTMDFLVQIFSFLLISASVAMSRGENVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - DNA, 435 bp, synthetic construct
  misc_feature 1..435: note = Description of sequence: light chain variable domain nucleotide sequence; Clone: 1-3C-F11
  source 1..435: mol_type = other DNA
atgagaagagaagtaattagccagggaacaaaattcaaatacacaatggattttctggtgcagattttcagcttcttgctaatcagtgcctcagttgcaatgtccagaggagaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttacccactaacgttcggaggggggaccaagctggaaataaaa

SEQ ID NO: 32 - protein, 145 aa, synthetic construct
  REGION 1..145: note = Description of sequence: light chain variable domain amino acid sequence; Clone: 1-3C-F11
MRREVISQGTKFKYTMDFLVQIFSFLLISASVAMSRGENVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGGGTKLEIK

SEQ ID NO: 33 - DNA, 1116 bp, synthetic construct
  misc_feature 1..1116: note = Description of sequence: heavy chain protein nucleotide sequence; Clone: 1-3C-F11
  source 1..1116: mol_type = other DNA
atggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctgggctgcaactggacgagacctgtgctgaggcccaggacggggagctggacgggctctggacgaccatcaccatcttcatcagcctcttcctgctcagcgtgtgctacagcgcctctgtcacactcttcaaggtaaagtggatcttctcctcggtggtggagctgaagcagacactggttcctgaatacaagaacatgattgggcaagcgccc

SEQ ID NO: 34 - protein, 372 aa, synthetic construct
  REGION 1..372: note = Description of sequence: heavy chain protein amino acid sequence; Clone: 1-3C-F11
MVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGLQLDETCAEAQDGELDGLWTTITIFISLFLLSVCYSASVTLFKVKWIFSSVVELKQTLVPEYKNMIGQAP

SEQ ID NO: 35 - protein, 7 aa, synthetic construct
  REGION 1..7: note = Description of sequence: CDRL1 amino acid sequence; Clone: 1-3C-F11
SSVSSSY

SEQ ID NO: 36 - skipped

SEQ ID NO: 37 - protein, 9 aa, synthetic construct
  REGION 1..9: note = Description of sequence: CDRL3 amino acid sequence; Clone: 1-3C-F11
QQYSGYPLT

SEQ ID NO: 38 - protein, 252 aa, synthetic construct
  REGION 1..252: note = Description of sequence: light chain protein humanized amino acid sequence; Clone: 1-3C-F11
MRREVISQGTKFKYTMDFLVQIFSFLLISASVAMSRGENVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 21 bp, synthetic construct
  misc_feature 1..21: note = Description of sequence: Primer sequence
  source 1..21: mol_type = other DNA
agcgcagtttgggatacagca

SEQ ID NO: 40 - DNA, 27 bp, synthetic construct
  misc_feature 1..27: note = Description of sequence: Primer sequence
  source 1..27: mol_type = other DNA
ctttaatgtgcccttatatacttcacc

SEQ ID NO: 41 - DNA, 25 bp, synthetic construct
  misc_feature 1..25: note = Description of sequence: TaqMan probe
  source 1..25: mol_type = other DNA
  modified_base 1: mod_base = OTHER; note = nulceotide bound to dye, 6-FAM dye
  modified_base 9^10: mod_base = OTHER; note = nulceotides bound to quencher, ZEN
  modified_base 25: mod_base = OTHER; note = nucleotide bound to quencher, Iowa black quencher
tcagaaacagcctatgagggaaatt

SEQ ID NO: 42 - DNA, 21 bp, synthetic construct
  misc_feature 1..21: note = Description of sequence: Primer sequence
  source 1..21: mol_type = other DNA
ctcaaactcctgaccccgtga

SEQ ID NO: 43 - DNA, 21 bp, synthetic construct
  misc_feature 1..21: note = Description of sequence: Primer sequence
  source 1..21: mol_type = other DNA
gaacacaaacccttagggggc

SEQ ID NO: 44 - DNA, 23 bp, synthetic construct
  misc_feature 1..23: note = Description of sequence: TaqMan probe
  source 1..23: mol_type = other DNA
  modified_base 1: mod_base = OTHER; note = nulceotide bound to dye, 6-FAM dye
  modified_base 9^10: mod_base = OTHER; note = nulceotides bound to quencher, ZEN
  modified_base 23: mod_base = OTHER; note = nucleotide bound to quencher, Iowa black quencher
ccaccttctagctattgagtagc

SEQ ID NO: 45 - protein, 34 aa, synthetic construct
  REGION 1..34: note = Description of sequence: Primer, cDNA: PDGFRA, Vector: pET28-HisTAG-Delta-PDGFRAaa1-528
AAGCTTGAATTCATGGGGACTTCCCATCCGGCGT

SEQ ID NO: 46 - protein, 35 aa, synthetic construct
  REGION 1..35: note = Description of sequence: Primer, cDNA: PDGFRA, Vector: pET28-HisTAG-Delta-PDGFRAaa1-528
TCTAGAGCGGCCGCAGCCACCGTGAGTTCAGAACG

SEQ ID NO: 47 - protein, 52 aa, synthetic construct
  REGION 1..52: note = Description of sequence: Primer, cDNA: PDGFRA, Vector: pCMV13-HisTAG-Delta-PDGFRAaa1-528
AAGCTTGAATTCCATCATCATCATCATCACATGGGGACTTCCCATCCGGCGT

SEQ ID NO: 48 - protein, 35 aa, synthetic construct; identical to SEQ ID NO: 46
  REGION 1..35: note = Description of sequence: Primer, cDNA: PDGFRA, Vector: pCMV13-HisTAG-Delta-PDGFRAaa1-528

SEQ ID NO: 49 - protein, 33 aa, synthetic construct
  REGION 1..33: note = Description of sequence: Primer, cDNA: MET, Vector: pET28-HisTAG-Delta-METaa1-646
AAGCTTGAATTCATGAAGGCCCCCGCTGTGCTT

SEQ ID NO: 50 - protein, 35 aa, synthetic construct
  REGION 1..35: note = Description of sequence: Primer, cDNA: MET, Vector: pET28-HisTAG-Delta-METaa1-646
TCTAGAGCGGCCGCTGTCCCGTGGCCATTTGAAAT

SEQ ID NO: 51 - protein, 51 aa, synthetic construct
  REGION 1..51: note = Description of sequence: Primer, cDNA: MET, Vector: pCMV13-HisTAG-Delta-METaa1-646
AAGCTTGAATTCCATCATCATCATCATCACATGAAGGCCCCCGCTGTGCTT

SEQ ID NO: 52 - protein, 35 aa, synthetic construct; identical to SEQ ID NO: 50
  REGION 1..35: note = Description of sequence: Primer, cDNA: MET, Vector: pCMV13-HisTAG-Delta-METaa1-646

SEQ ID NO: 53 - protein, 33 aa, synthetic construct
  REGION 1..33: note = Description of sequence: Primer, cDNA: AXL, Vector: pET28-HisTAG-Delta-AXLaa1-451
AAGCTTGAATTCATGGCGTGGCGGTGCCCCAGG

SEQ ID NO: 54 - protein, 35 aa, synthetic construct
  REGION 1..35: note = Description of sequence: Primer, cDNA: AXL, Vector: pET28-HisTAG-Delta-AXLaa1-451
TCTAGAGCGGCCGCCCACCAGGGCCACGAGAAGGC

SEQ ID NO: 55 - protein, 51 aa, synthetic construct
  REGION 1..51: note = Description of sequence: Primer, cDNA: AXL, Vector: pCMV13-HisTAG-Delta-AXLaa1-451
AAGCTTGAATTCCATCATCATCATCATCACATGGCGTGGCGGTGCCCCAGG

SEQ ID NO: 56 - protein, 35 aa, synthetic construct; identical to SEQ ID NO: 54
  REGION 1..35: note = Description of sequence: Primer, cDNA: AXL, Vector: pCMV13-HisTAG-Delta-AXLaa1-451

SEQ ID NO: 57 - protein, 33 aa, synthetic construct
  REGION 1..33: note = Description of sequence: Primer, cDNA: CDH2, Vector: pET28-HisTAG-Delta-CDH2aa1-724
AAGCTTGAATTCATGTGCCGGATAGCGGGAGCG

SEQ ID NO: 58 - protein, 35 aa, synthetic construct
  REGION 1..35: note = Description of sequence: Primer, cDNA: CDH2, Vector: pET28-HisTAG-Delta-CDH2aa1-724
TCTAGAGCGGCCGCGGCACCGGTGCCAAGCCCCGC

SEQ ID NO: 59 - protein, 51 aa, synthetic construct
  REGION 1..51: note = Description of sequence: Primer, cDNA: CDH2, Vector: pCMV13-HisTAG-Delta-CDH2aa1-724
AAGCTTGAATTCCATCATCATCATCATCACATGTGCCGGATAGCGGGAGCG

SEQ ID NO: 60 - protein, 35 aa, synthetic construct; identical to SEQ ID NO: 58
  REGION 1..35: note = Description of sequence: Primer, cDNA: CDH2, Vector: pCMV13-HisTAG-Delta-CDH2aa1-724

SEQ ID NO: 61 - protein, 21 aa, synthetic construct
  REGION 1..21: note = Description of sequence: Peptide containing tyrosine position. Gene: PDGFR, Peptide No.: 1, Tyrosine position: Y59
CFGESEVSWQYPMSEEESSDV

SEQ ID NO: 62 - protein, 21 aa, synthetic construct
  REGION 1..21: note = Description of sequence: Peptide containing tyrosine position. Gene: PDGFR, Peptide No.: 2, Tyrosine position: Y98/Y101-2
SSASAAHTGLYTCYYNHTQTE

SEQ ID NO: 63 - protein, 21 aa, synthetic construct
  REGION 1..21: note = Description of sequence: Peptide containing tyrosine position. Gene: PDGFR, Peptide No.: 3, Tyrosine position: Y118/Y120
EENELEGRHIYIYVPDPDVAF

SEQ ID NO: 64 - protein, 21 aa, synthetic construct
  REGION 1..21: note = Description of sequence: Peptide containing tyrosine position. Gene: PDGFR, Peptide No.: 4, Tyrosine position: Y136
VAFVPLGMTDYLVIVEDDDSA

SEQ ID NO: 65 - protein, 21 aa, synthetic construct
  REGION 1..21: note = Description of sequence: Peptide containing tyrosine position. Gene: PDGFR, Peptide No.: 5, Tyrosine position: Y172
HNSEGVVPASYDSRQGFNGTF

SEQ ID NO: 66 - protein, 21 aa, synthetic construct
  REGION 1..21: note = Description of sequence: Peptide containing tyrosine position. Gene: PDGFR, Peptide No.: 6, Tyrosine position: Y187
GFNGTFTVGPYICEATVKGKK

SEQ ID NO: 67 - protein, 21 aa, synthetic construct
  REGION 1..21: note = Description of sequence: Peptide containing tyrosine position. Gene: PDGFR, Peptide No.: 7, Tyrosine position: Y206
KKFQTIPFNVYALKATSELDL

SEQ ID NO: 68 - protein, 21 aa, synthetic construct
  REGION 1..21: note = Description of sequence: Peptide containing tyrosine position. Gene: PDGFR, Peptide No.: 8, Tyrosine position: Y225
DLEMEALKTVYKSGETIVVTC

SEQ ID NO: 69 - protein, 21 aa, synthetic construct
  REGION 1..21: note = Description of sequence: Peptide containing tyrosine position. Gene: PDGFR, Peptide No.: 9, Tyrosine position: Y249
NNEVVDLQWTYPGEVKGKGIT

SEQ ID NO: 70 - protein, 21 aa, synthetic construct
  REGION 1..21: note = Description of sequence: Peptide containing tyrosine position. Gene: PDGFR, Peptide No.: 10, Tyrosine position: Y273
EIKVPSIKLVYTLTVPEATVK

SEQ ID NO: 71 - protein, 21 aa, synthetic construct
  REGION 1..21: note = Description of sequence: Peptide containing tyrosine position. Gene: PDGFR, Peptide No.: 11, Tyrosine position: Y288
PEATVKDSGDYECAARQATRE

SEQ ID NO: 72 - protein, 21 aa, synthetic construct
  REGION 1..21: note = Description of sequence: Peptide containing tyrosine position. Gene: PDGFR, Peptide No.: 12, Tyrosine position: Y342
VKHFVVEVRAYPPPRISWLKN

SEQ ID NO: 73 - protein, 21 aa, synthetic construct
  REGION 1..21: note = Description of sequence: Peptide containing tyrosine position. Gene: PDGFR, Peptide No.: 13, Tyrosine position: Y375
TDVEKIQEIRYRSKLKLIRAK

SEQ ID NO: 74 - protein, 21 aa, synthetic construct
  REGION 1..21: note = Description of sequence: Peptide containing tyrosine position. Gene: PDGFR, Peptide No.: 14, Tyrosine position: Y391
IRAKEEDSGHYTIVAQNEDAV

SEQ ID NO: 75 - protein, 21 aa, synthetic construct
  REGION 1..21: note = Description of sequence: Peptide containing tyrosine position. Gene: PDGFR, Peptide No.: 15, Tyrosine position: Y405
VAQNEDAVKSYTFELLTQVPS

SEQ ID NO: 76 - protein, 21 aa, synthetic construct
  REGION 1..21: note = Description of sequence: Peptide containing tyrosine position. Gene: MET, Peptide No.: 1, Tyrosine position: Y41
AKSEMNVNMKYQLPNFTAETP

SEQ ID NO: 77 - protein, 21 aa, synthetic construct
  REGION 1..21: note = Description of sequence: Peptide containing tyrosine position. Gene: MET, Peptide No.: 2, Tyrosine position: Y69/Y71
EHHIFLGATNYIYVLNEEDLQ

SEQ ID NO: 78 - protein, 21 aa, synthetic construct
  REGION 1..21: note = Description of sequence: Peptide containing tyrosine position. Gene: MET, Peptide No.: 3, Tyrosine position: Y84
NEEDLQKVAEYKTGPVLEHPD

SEQ ID NO: 79 - protein, 21 aa, synthetic construct
  REGION 1..21: note = Description of sequence: Peptide containing tyrosine position. Gene: MET, Peptide No.: 4, Tyrosine position: Y125/Y126
NINMALVVDTYYDDQLISCGS

SEQ ID NO: 80 - protein, 21 aa, synthetic construct
  REGION 1..21: note = Description of sequence: Peptide containing tyrosine position. Gene: MET, Peptide No.: 5, Tyrosine position: Y205
FFVGNTINSSYFPDHPLHSIS

SEQ ID NO: 81 - protein, 21 aa, synthetic construct
  REGION 1..21: note = Description of sequence: Peptide containing tyrosine position. Gene: MET, Peptide No.: 6, Tyrosine position: Y234
DGFMFLTDQSYIDVLPEFRDS

SEQ ID NO: 82 - protein, 21 aa, synthetic construct
  REGION 1..21: note = Description of sequence: Peptide containing tyrosine position. Gene: MET, Peptide No.: 7, Tyrosine position: Y245/Y249
IDVLPEFRDSYPIKYVHAFES

SEQ ID NO: 83 - protein, 21 aa, synthetic construct
  REGION 1..21: note = Description of sequence: Peptide containing tyrosine position. Gene: MET, Peptide No.: 8, Tyrosine position: Y260
VHAFESNNFIYFLTVQRETLD

SEQ ID NO: 84 - protein, 21 aa, synthetic construct
  REGION 1..21: note = Description of sequence: Peptide containing tyrosine position. Gene: MET, Peptide No.: 9, Tyrosine position: Y291
FCSINSGLHSYMEMPLECILT

SEQ ID NO: 85 - protein, 21 aa, synthetic construct
  REGION 1..21: note = Description of sequence: Peptide containing tyrosine position. Gene: MET, Peptide No.: 10, Tyrosine position: Y321
KEVFNILQAAYVSKPGAQLAR

SEQ ID NO: 86 - protein, 21 aa, synthetic construct
  REGION 1..21: note = Description of sequence: Peptide containing tyrosine position. Gene: MET, Peptide No.: 11, Tyrosine position: Y369
RSAMCAFPIKYVNDFFNKIVN

SEQ ID NO: 87 - protein, 21 aa, synthetic construct
  REGION 1..21: note = Description of sequence: Peptide containing tyrosine position. Gene: MET, Peptide No.: 12, Tyrosine position: Y390
KNNVRCLQHFYGPNHEHCFNR

SEQ ID NO: 88 - protein, 21 aa, synthetic construct
  REGION 1..21: note = Description of sequence: Peptide containing tyrosine position. Gene: MET, Peptide No.: 13, Tyrosine position: Y416
SSGCEARRDEYRTEFTTALQR

SEQ ID NO: 89 - protein, 21 aa, synthetic construct
  REGION 1..21: note = Description of sequence: Peptide containing tyrosine position. Gene: MET, Peptide No.: 14, Tyrosine position: Y501
IVEHTLNQNGYTLVITGKKIT

SEQ ID NO: 90 - protein, 21 aa, synthetic construct
  REGION 1..21: note = Description of sequence: Peptide containing tyrosine position. Gene: MET, Peptide No.: 15, Tyrosine position: Y566
WTQQICLPAIYKVFPNSAPLE

SEQ ID NO: 91 - protein, 25 aa, synthetic construct
  REGION 1..25: note = Description of sequence: Peptide containing tyrosine position. Gene: AXL, Peptide No.: 1, Tyrosine position: Y115
RITSLQLSDTGQYQCLVFLGHQTFV

SEQ ID NO: 92 - protein, 24 aa, synthetic construct
  REGION 1..24: note = Description of sequence: Peptide containing tyrosine position. Gene: AXL, Peptide No.: 2, Tyrosine position: Y132
QCLVFLGHQTFVSQPGYVGLEGLP

SEQ ID NO: 93 - protein, 25 aa, synthetic construct
  REGION 1..25: note = Description of sequence: Peptide containing tyrosine position. Gene: AXL, Peptide No.: 3, Tyrosine position: Y140
VGLEGLPYFLEEPEDRTVAANTPFN

SEQ ID NO: 94 - protein, 25 aa, synthetic construct
  REGION 1..25: note = Description of sequence: Peptide containing tyrosine position. Gene: AXL, Peptide No.: 4, Tyrosine position: Y253
LEVAWTPGLSGIYPLTHCTLQAVLS

SEQ ID NO: 95 - protein, 25 aa, synthetic construct
  REGION 1..25: note = Description of sequence: Peptide containing tyrosine position. Gene: AXL, Peptide No.: 5, Tyrosine position: Y367/Y371
RAPLQGTLLGYRLAYQGQDTPEVLM

SEQ ID NO: 96 - protein, 25 aa, synthetic construct
  REGION 1..25: note = Description of sequence: Peptide containing tyrosine position. Gene: AXL, Peptide No.: 6, Tyrosine position: Y409
GSVSNLTVCVAAYTAAGDGPWSLPV

SEQ ID NO: 97 - protein, 21 aa, synthetic construct
  REGION 1..21: note = Description of sequence: Peptide containing tyrosine position. Gene: CDH2, Peptide No.: 1, Tyrosine position: Y40
LCKTGFPEDVYSAVLSKDVHE

SEQ ID NO: 98 - protein, 21 aa, synthetic construct
  REGION 1..21: note = Description of sequence: Peptide containing tyrosine position. Gene: CDH2, Peptide No.: 2, Tyrosine position: Y70
SNCNGKRKVQYESSEPADFKV

SEQ ID NO: 99 - protein, 21 aa, synthetic construct
  REGION 1..21: note = Description of sequence: Peptide containing tyrosine position. Gene: CDH2, Peptide No.: 3, Tyrosine position: Y87
DFKVDEDGMVYAVRSFPLSSE

SEQ ID NO: 100 - protein, 21 aa, synthetic construct
  REGION 1..21: note = Description of sequence: Peptide containing tyrosine position. Gene: CDH2, Peptide No.: 4, Tyrosine position: Y104
LSSEHAKFLIYAQDKETQEKW

SEQ ID NO: 101 - protein, 21 aa, synthetic construct
  REGION 1..21: note = Description of sequence: Peptide containing tyrosine position. Gene: CDH2, Peptide No.: 5, Tyrosine position: Y194
SDRDKNLSLRYSVTGPGADQP

SEQ ID NO: 102 - protein, 21 aa, synthetic construct
  REGION 1..21: note = Description of sequence: Peptide containing tyrosine position. Gene: CDH2, Peptide No.: 6, Tyrosine position: Y284
TVPEGSKPGTYVMTVTAIDAD

SEQ ID NO: 103 - protein, 21 aa, synthetic construct
  REGION 1..21: note = Description of sequence: Peptide containing tyrosine position. Gene: CDH2, Peptide No.: 7, Tyrosine position: Y305
DPNALNGMLRYRIVSQAPSTP

SEQ ID NO: 104 - protein, 21 aa, synthetic construct
  REGION 1..21: note = Description of sequence: Peptide containing tyrosine position. Gene: CDH2, Peptide No.: 8, Tyrosine position: Y344
AGLDREKVQQYTLIIQATDME

SEQ ID NO: 105 - protein, 21 aa, synthetic construct
  REGION 1..21: note = Description of sequence: Peptide containing tyrosine position. Gene: CDH2, Peptide No.: 9, Tyrosine position: Y359
QATDMEGNPTYGLSNTATAVI

SEQ ID NO: 106 - protein, 21 aa, synthetic construct
  REGION 1..21: note = Description of sequence: Peptide containing tyrosine position. Gene: CDH2, Peptide No.: 10, Tyrosine position: Y387
NPPEFTAMTFYGEVPENRVDI

SEQ ID NO: 107 - protein, 21 aa, synthetic construct
  REGION 1..21: note = Description of sequence: Peptide containing tyrosine position. Gene: CDH2, Peptide No.: 11, Tyrosine position: Y419
QPHTPAWNAVYRISGGDPTGR

SEQ ID NO: 108 - protein, 21 aa, synthetic construct
  REGION 1..21: note = Description of sequence: Peptide containing tyrosine position. Gene: CDH2, Peptide No.: 12, Tyrosine position: Y495
VTVIDVNENPYFAPNPKIIRQ

SEQ ID NO: 109 - protein, 21 aa, synthetic construct
  REGION 1..21: note = Description of sequence: Peptide containing tyrosine position. Gene: CDH2, Peptide No.: 13, Tyrosine position: Y526/Y533
TTFTAQDPDRYMQQNIRYTKL

SEQ ID NO: 110 - protein, 21 aa, synthetic construct
  REGION 1..21: note = Description of sequence: Peptide containing tyrosine position. Gene: CDH2, Peptide No.: 14, Tyrosine position: Y526/Y533
PDRYMQQNIRYTKLSDPANWL

SEQ ID NO: 111 - protein, 21 aa, synthetic construct
  REGION 1..21: note = Description of sequence: Peptide containing tyrosine position. Gene: CDH2, Peptide No.: 15, Tyrosine position: Y570
RESPNVKNNIYNATFLASDNG

SEQ ID NO: 112 - protein, 21 aa, synthetic construct
  REGION 1..21: note = Description of sequence: Peptide containing tyrosine position. Gene: CDH2, Peptide No.: 16, Tyrosine position: Y593
PMSGTGTLQIYLLDINDNAPQ

SEQ ID NO: 113 - protein, 21 aa, synthetic construct
  REGION 1..21: note = Description of sequence: Peptide containing tyrosine position. Gene: CDH2, Peptide No.: 17, Tyrosine position: Y627
PNSINITALDYDIDPNAGPFA

SEQ ID NO: 114 - protein, 21 aa, synthetic construct
  REGION 1..21: note = Description of sequence: Peptide containing tyrosine position. Gene: CDH2, Peptide No.: 18, Tyrosine position: Y675
LKIKFLEAGIYEVPIIITDSG

SEQ ID NO: 115 - protein, 25 aa, synthetic construct
  REGION 1..25: note = Description of sequence: Phosophorated peptide: Peptide No.: 1, Position of phosphorylation: pY118/pY120
  MOD_RES 13: note = Phosophorylated amino acid
QTEENELEGRHIYIYVPDPDVAFVP

SEQ ID NO: 116 - protein, 25 aa, synthetic construct
  REGION 1..25: note = Description of sequence: Phosophorated peptide: Peptide No.: 2, Position of phosphorylation: pY118/pY120
  MOD_RES 13: note = Phosophorylated amino acid
QTEENELEGRHIYIYVPDPDVAFVP

SEQ ID NO: 117 - protein, 25 aa, synthetic construct
  REGION 1..25: note = Description of sequence: Phosophorated peptide: Peptide No.: 3, Position of phosphorylation: pY120
  MOD_RES 15: note = Phosophorylated amino acid
QTEENELEGRHIYIYVPDPDVAFVP

SEQ ID NO: 118 - protein, 23 aa, synthetic construct
  REGION 1..23: note = Description of sequence: Phosophorated peptide: Peptide No.: 4, Position of phosphorylation: pY288
  MOD_RES 11: note = Phosophorylated amino acid
PEATVKDSGDYECAARQATREVK

SEQ ID NO: 119 - protein, 23 aa, synthetic construct
  REGION 1..23: note = Description of sequence: Peptide: Peptide No.: 5, Position of potential phosphorylation: Y288
PEATVKDSGDYECAARQATREVK

SEQ ID NO: 120 - protein, 21 aa, synthetic construct
  REGION 1..21: note = Description of sequence: Phosophorated peptide: Peptide No.: 6, Position of phosphorylation: pY342
  MOD_RES 11: note = Phosophorylated amino acid
VKHFVVEVRAYPPPRISWLKN

SEQ ID NO: 121 - protein, 21 aa, synthetic construct
  REGION 1..21: note = Description of sequence: Peptide: Peptide No.: 7, Position of potential phosphorylation: Y342
VKHFVVEVRAYPPPRISWLKN

SEQ ID NO: 122 - protein, 21 aa, synthetic construct
  REGION 1..21: note = Description of sequence: Peptide: Peptide No.: 8, Position of potential phosphorylation: Y375
TDVEKIQEIRYRSKLKLIRAK

SEQ ID NO: 123 - protein, 21 aa, synthetic construct
  REGION 1..21: note = Description of sequence: Phosophorated peptide: Peptide No.: 9, Position of phosphorylation: pY375
  MOD_RES 11: note = Phosophorylated amino acid
TDVEKIQEIRYRSKLKLIRAK

SEQ ID NO: 124 - protein, 21 aa, synthetic construct
  REGION 1..21: note = Description of sequence: Phosophorated peptide: Peptide No.: 10, Position of phosphorylation: pY391
  MOD_RES 11: note = Phosophorylated amino acid
IRAKEEDSGHYTIVAQNEDAV

SEQ ID NO: 125 - protein, 21 aa, synthetic construct
  REGION 1..21: note = Description of sequence: Peptide: Peptide No.: 11, Position of potential phosphorylation: Y391
IRAKEEDSGHYTIVAQNEDAV

SEQ ID NO: 126 - protein, 25 aa, synthetic construct
  REGION 1..25: note = Description of sequence: Peptide: Peptide No.: 12, Position of potential phosphorylation: Y118/Y119
QTEENELEGRHIYIYVPDPDVAFVP

(19) United States
(12) Patent Application Publication
(10) Pub. No.: US 2026/0258142 A1
(43) Pub. Date: Sep. 3, 2026

(54) IMMUNO-TARGETING THE ECTOPIC PHOSPHORYLATION SITES OF PDGFRA GENERATED BY MAN2A1-FER FUSION IN HEPATOCELLULAR CARCINOMA

(71) Applicant: UNIVERSITY OF PITTSBURGH - OF THE COMMONWEALTH SYSTEM OF HIGHER EDUCATION, Pittsburgh, PA (US)

(72) Inventors: Jianhua LUO, Wexford, PA (US); Muhamuda KADER, Pittsburgh, PA (US); Yanping YU, Wexford, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/671,865

(22) Filed: May 8, 2026

Related U.S. Application Data
(63) Continuation of application No. PCT/US2024/054943, filed on Nov. 7, 2024.
(60) Provisional application No. 63/597,144, filed on Nov. 8, 2023.

(51) Int. Cl. C07K 16/28; A61K 9/51; A61K 38/00; A61P 35/00

(52) U.S. Cl. CPC C07K 16/2863; A61K 9/5123; A61K 38/00; A61P 35/00; C07K 2317/21; C07K 2317/24; C07K 2317/54; C07K 2317/55; C07K 2317/565

(57) ABSTRACT

The presently disclosed subject matter relates to antibodies and antigen-binding fragments that bind specifically to phosphorylated PDGFRA, and methods of treating cancer expressing MAN2A1-FER which ectopically phosphorylates PDGFRA. MAN2A1-FER can be expressed in liver cancer, prostate cancer, brain cancer, glioblastoma multiforme, breast cancer, lung cancer, non-small cell lung cancer, colon cancer, and renal cell carcinoma.

### CROSS-REFERENCE TO RELATED APPLICATIONS

This application is a continuation of International Patent Application No. PCT/US2024/054943, filed Nov. 7, 2024, which claims priority to U.S. Provisional Application No. 63/597,144, filed Nov. 8, 2023, the content of both of which are incorporated by reference in their entireties.

### GRANT INFORMATION

This invention was made with government support under CA229262, and DK120531 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 6, 2024, is entitled “0723961038_ST26.xml”, and is 183,770 bytes in size.

### INTRODUCTION

The presently disclosed subject matter relates to antibodies and antigen-binding fragments that bind specifically to phosphorylated PDGFRA. The presently disclosed subject matter further relates to methods of treating cancer expressing MAN2A1-FER which ectopically phosphorylates PDGFRA.

### BACKGROUND

Liver cancer is one of the most lethal human malignancies. Worldwide, 830,200 people died from primary liver cancers in 2020. It is expected the mortality of primary liver cancer will rise by more than 55% by 2040. Among the primary liver cancers, hepatocellular carcinoma accounted for 90% of the liver cancer cases. Currently, the main approach in treating primary liver cancer is surgical interventions, either through liver transplantation or surgical resection. However, these options are only available to early-stage liver cancer patients. Most liver cancers have insidious clinical courses. Many patients are at the advanced stages of liver cancer at the time of diagnosis. Even though extensive progress was made in cancer treatment through molecular targeting and immune modification, few effective treatments were developed for late-stage liver cancers.

FER tyrosine kinase is a downstream signaling molecule for several growth factor receptors and plays a role in cell-cell adhesion. In previous studies, it was observed that the C-terminus of FER kinase fuses with the N-terminus of Mannosidase alpha class 2A member 1 (MAN2A1) and generates a chimera protein called MAN2A1-FER. The fusion genes were detected in 15% to 80% of hepatocellular carcinoma patients. The fusion protein retained the kinase domain of FER and displayed 3.8-fold kinase activity of the native FER protein. The introduction of MAN2A1-FER increased cancer cell proliferation in vitro and promoted cancer growth, invasion, and metastasis in xenografted liver cancer animal models. When coupled with somatic deletion of Pten, MAN2A1-FER expression induced spontaneous liver cancer in mice. Despite the obvious oncogenic activity of MAN2A1-FER, the mechanism of its tumor-promoting activity remains unclear. MAN2A1-FER chimera protein is translocated to the Golgi apparatus and ectopically phosphorylates the N-terminus of epidermal growth factor receptor (EGFR). There is need to investigate the role of MAN2A1-FER in ectopic phosphorylation of membrane proteins and activation of signaling cascades, and to identify monoclonal antibodies for targeting ectopic phosphorylation sites.

### SUMMARY OF THE INVENTION

The presently disclosed subject matter provides an antibody or antigen-binding fragment, wherein the antibody or antigen-binding fragment specifically binds to at least one phosphotyrosine in the extracellular domain of PDGFRA. In certain embodiments the heavy/light chain protein sequences comprise amino acid sequences that are at least 95% identical to the amino acid sequences selected from the group consisting of SEQ ID NO.: 6/SEQ ID NO.: 2, SEQ ID NO.: 13/SEQ ID NO.: 12, SEQ ID NO.: 13/SEQ ID NO.: 14, SEQ ID NO.: 24/SEQ ID NO.: 20, SEQ ID NO.: 24/SEQ ID NO.: 28, SEQ ID NO.: 34/SEQ ID NO.: 30, and SEQ ID NO.: 34/SEQ ID NO.: 38. In certain embodiments, the heavy/light chain protein sequences comprise amino acid sequences selected from the group consisting of SEQ ID NO.: 6/SEQ ID NO.: 2, SEQ ID NO.: 13/SEQ ID NO.: 12, SEQ ID NO.: 13/SEQ ID NO.: 14, SEQ ID NO.: 24/SEQ ID NO.: 20, SEQ ID NO.: 24/SEQ ID NO.: 28, SEQ ID NO.: 34/SEQ ID NO.: 30, and SEQ ID NO.: 34/SEQ ID NO.: 38. In certain embodiments, the heavy/light chain variable domain sequences comprise amino acid sequences that are at least 95% identical to the amino acid sequences selected from the group consisting of SEQ ID NO.: 8/SEQ ID NO.: 4 and SEQ ID NO.: 8/SEQ ID NO.: 15. In certain embodiments, the heavy/light chain variable domain sequences comprise amino acid sequences selected from the group consisting of SEQ ID NO.: 8/SEQ ID NO.: 4 and SEQ ID NO.: 8/SEQ ID NO.: 15. In certain embodiments, the light chain CDRs comprise amino acid sequences that are at least 95% identical to SEQ ID NO.: 9, SEQ ID NO.: 10, and SEQ ID NO.: 11; the light chain CDRs comprise amino acid sequences that are at least 95% identical to SEQ ID NO.: 16, SEQ ID NO.: 17, and SEQ ID NO.: 18; the light chain CDRs comprise amino acid sequences that are at least 95% identical to SEQ ID NO.: 25, SEQ ID NO.: 26, and SEQ ID NO.: 27; or the light chain CDRs comprise amino acid sequences that are at least 95% identical to SEQ ID NO.: 35, SEQ ID NO.: 36, and SEQ ID NO.: 37. In certain embodiments, the light chain CDRs comprise amino acid sequences that are identical to SEQ ID NO.: 9, SEQ ID NO.: 10, and SEQ ID NO.: 11; the light chain CDRs comprise amino acid sequences that are identical to SEQ ID NO.: 16, SEQ ID NO.: 17, and SEQ ID NO.: 18; the light chain CDRs comprise amino acid sequences that are identical to SEQ ID NO.: 25, SEQ ID NO.: 26, and SEQ ID NO.: 27; or the light chain CDRs comprise amino acid sequences that are identical to SEQ ID NO.: 35, SEQ ID NO.: 36, and SEQ ID NO.: 37.

In certain embodiments, the antibody or antigen-binding fragment comprises a heavy chain variable domain sequence that is truncated. In certain embodiments, the antibody or antigen-binding fragment comprises a heavy chain variable domain sequence that is truncated to remove one or more heavy chain complementarity determining regions. In certain embodiments, the antibody or antigen-binding fragment comprises a heavy chain variable domain sequence that is truncated to remove all three heavy chain complementarity determining regions. In certain embodiments, the antibody or antigen-binding fragment comprises one, two, or three heavy chain complementarity determining regions. In certain embodiments, the antibody or antigen-binding fragment does not have heavy chain complementarity determining regions.

In certain embodiments, the antibody or antigen-binding fragment specifically binds to at least one phosphotyrosine selected from the list consisting of pY118/pY120, pY118, pY120, pY342, pY375, and pY391. In certain embodiments, the antibody or antigen-binding fragment specifically binds to pY118/pY120. In certain embodiments, the antibody or antigen-binding fragment does not bind to pY118, pY120, pY342, pY375, or pY391. In certain embodiments, the antibody or antigen-binding fragment does not bind to unphosphorylated extracellular domain of PDGFRA. In certain embodiments, the antigen-binding fragment is a Fv, Fab, Fab′, or F(ab′)₂. In certain embodiments, the antibody or antigen-binding fragment is conjugated to an anti-cancer agent.

The presently disclosed subject matter provides a method of treating cancer, comprising administering a therapeutically effective amount of the antibody or antigen-binding fragment disclosed herein, to a subject in need thereof. In certain embodiments, the cancer expresses MAN2A1-FER. In certain embodiments, the cancer is selected from the group consisting of liver cancer, prostate cancer, brain cancer, glioblastoma multiforme, breast cancer, lung cancer, non-small cell lung cancer, colon cancer, and renal cell carcinoma. In certain embodiments, the method further comprises determining the presence of MAN2A1-FER in a sample obtained from the subject. In certain embodiments, the sample is selected from the group consisting of cells in culture, cell supernatants, cell lysates, serum, blood plasma, blood, plasma, stool, urine, lymphatic fluid, cerebrospinal fluid, ascites, ductal lavage, saliva, fresh tissue, frozen tissue, preserved tissue, biopsy, or aspirate, cerebral spinal fluid, amniotic fluid, peritoneal fluid, interstitial fluid, and combinations thereof.

The presently disclosed subject matter provides a method of treating cancer in a subject in need thereof, comprising obtaining a sample from the subject; determining whether a subject is at increased risk of manifesting progressive cancer comprising determining whether the sample contains a fusion gene by contacting the sample with the antibody or antigen-binding fragment disclosed herein; determining that the patient is at increased risk of progressive cancer when the sample contains the fusion gene; and treating the patient that is at increased risk of progressive cancer. In certain embodiments, the patient comprises administering a therapeutically effective amount of the antibody or antigen-binding fragment of disclosed herein. In certain embodiments, the method further comprises performing molecular imaging of the subject using a tracer compound that is conjugated to the antibody or antigen-binding fragment disclosed herein. In certain embodiments, the method further comprises performing frequent monitoring for recurrence or metastasis by ultrasound imaging, CT imaging, MRI imaging, PET scan, a radiotherapy, a chemotherapy, and/or an antibody. In certain embodiments, the fusion gene is MAN2A1-FER. In certain embodiments, the sample is selected from the group consisting of cells in culture, cell supernatants, cell lysates, serum, blood plasma, blood, plasma, stool, urine, lymphatic fluid, cerebrospinal fluid, ascites, ductal lavage, saliva, fresh tissue, frozen tissue, preserved tissue, biopsy, or aspirate, cerebral spinal fluid, amniotic fluid, peritoneal fluid, interstitial fluid, and combinations thereof.

The presently disclosed subject matter provides a kit comprising means for detecting the presence of MAN2A1-FER, comprising the antibody or antigen-binding fragment disclosed herein. In certain embodiments, the antibody or antigen-binding fragment does not bind to pY118, pY120, pY342, pY375, or pY391. In certain embodiments, the antibody or antigen-binding fragment specifically binds to pY118/pY120. In certain embodiments, the antibody or antigen-binding fragment does not bind to unphosphorylated extracellular domain of PDGFRA. In certain embodiments, the antigen-binding fragment is a Fv, Fab, Fab′, or F(ab′)₂.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIGS. 1A-1C show frequent expression of MAN2A1-FER in liver cancer and other cancer cell lines. FIG. 1A shows detection of MAN2A1-FER fusion transcripts in cell lines of HCC, Breast cancer (Brst Ca), colon cancer (Cca), renal cell carcinoma (RCC), glioblastoma multiforme (GBM), non-small cell lung cancer (NSCLC) and prostate cancer (Pca). FIG. 1B shows detection of the breakpoint sequence in the genomes of cancer cell lines as of FIG. 1A. FIG. 1C shows detection of MAN2A1-FER fusion protein in the cell lines by rabbit polyclonal antibodies specific for the C-terminus of FER. C denotes control cells negative for MAN2A1-FER.

FIGS. 2A-2F show that MAN2A1-FER phosphorylated the extracellular domains of EGFR, PDGFR, MET, AXL, and CDH2 in vitro and in vivo. FIG. 2A shows in vitro kinase assays of GST-FER and GST-MAN2A1-FER on substrates Poly E:Y (1:4), HisTAG-ΔEGFR^{aa1-650}, His TAG-ΔPDGFRA^{aa1-528}, HisTAG-ΔCDH2^{aa1-724}, HisTAG-ΔAXL^{aa1-451}, or HisTAG-ΔMET^{aa1-646}. FIG. 2B shows kinase assays of GST-MAN2A1-FER on tyrosine residue containing peptides (Table 3) corresponding to PDGFRA extracellular domain. FIG. 2C shows kinase assays of GST-MAN2A1-FER on tyrosine residue containing peptides (Table 3) corresponding to AXL extracellular domain. FIG. 2D shows kinase assays of GST-MAN2A1-FER on tyrosine residue containing peptides (Table 3) corresponding to CDH2 extracellular domain. FIG. 2E shows kinase assays of GST-MAN2A1-FER on tyrosine residue containing peptides (Table 3) corresponding to MET extracellular domain. FIG. 2F shows MAN2A1-FER phosphorylated the extracellular domains of CDH2, MET, AXL, and PDGFR in vivo. The His TAG-ΔPDGFRA^{aa1-528}, HisTAG-ΔCDH2^{aa1-724}, HisTAG-ΔAXL^{aa1-451} or HisTAG-ΔMET^{aa1-646} proteins were expressed through pCMV13 recombination constructs in HUH7 or HUH7ko cells where MAN2A1-FER was disrupted. The recombinant proteins were purified by HisTAG column. The proteins were then immunoblotted with antibodies specific for the recombinant proteins (top) and the phosphotyrosine (bottom).

FIGS. 3A-3B show that MANA1-FER activated the signaling cascades of PDGFR, EMT, AXL, and CDH2. FIG. 3A shows immunoblotting of indicated proteins and phospho-proteins in HEP3B cells transformed with pCNDA4-MAN2A1-FER-FLAG/pCDNA6TO and induced with or without tetracycline. FIG. 3B shows immunoblotting of indicated proteins and phospho-proteins in HUH7 cells or HUH7ko cells where MAN2A1-FER expression was disrupted by CRISPR-cas9 editing.

FIGS. 4A-4D show binding epitopes and binding affinity of antibodies from hybridoma clones 2-3B-G8, 1-3C-F11, 2-3C-C5, 2-6B-D8, and 1-4B-B10. FIG. 4A shows antibodies from clones 2-3B-G8, 1-3C-F11, 2-3C-C5, 2-6B-D8, and 1-4B-B10 bound phosphorylated HisTAG-ΔPDGFRA^{aa1-528} from HUH7 but not the unphosphorylated counterparts from E. coli or HUH7ko. FIG. 4B shows antibodies from clones 2-3B-G8, 1-3C-F11, 2-3C-C5, 2-6B-D8, and 1-4B-B10 bound to phosphorylated HisTAG-ΔPDGFRA^{aa1-528} with high affinity. Binding assays between the indicated antibodies and the phosphorylated HisTAG-ΔPDGFRA^{aa1-528} from HUH7 were performed. The dissociation constant (Kd) was indicated for each antibody. FIG. 4C shows antibodies from clones 2-3B-G8, 1-3C-F11, 2-3C-C5, 2-6B-D8, and 1-4B-B10 bound to specific phosphorylated tyrosine in the extracellular domain of PDGFRA. Immune dot blot analyses were performed on the tyrosine-phosphorylated peptides and their corresponding unphosphorylated controls (Table 4) with the indicated antibodies. FIG. 4D shows mapping of the antibody epitopes of 2-3B-G8, 1-3C-F11, 2-3C-C5, 2-6B-D8, and 1-4B-B10 in vivo. The protein extract of HUH7 transformed with pCMV13-HisTAG-ΔPDGFRA^{aa1-528} was partially digested with benzoic acid. The digested HisTAG-ΔPDGFRA^{aa1-528} fragments were purified by HisTAG column. Immunoblotting was performed after the protein fragments were resolved in 15% SDS-PAGE. HisTAG-ΔPDGFRA^{aa1-528} protein was indicated by blue ball (HisTAG) and bar (ΔPDGFRA^{aa1-528}). The benzoic acid cleavage (tryptophan) positions in HisTAG-ΔPDGFRA^{aa1-528} were indicated by red arrows. The specific antibody epitope was indicated by green vertical flipped Y.

FIG. 5 shows antibodies from hybridoma clones 1-3C-F11, 2-3C-C5, 2-6B-D8, and 1-4B-B10 bound MAN2A1-FER positive cells in vivo. Left: Images of immunofluorescence staining of HUH7 or HUH7ko cells where MAN2A1-FER was disrupted with IgG or antibodies from hybridoma clones 1-3C-F11 (F11, top), 2-3C-C5 (C5, second from the top), 1-4B-B10 (B10, third from the top), and 2-6B-D8 (D8, bottom). Right: Flow cytometry analyses of the immunofluorescence staining from the left.

FIGS. 6A-6E show that antibodies from hybridoma clone 2-3B-G8 inhibited PDGFRA activation, caused cell growth arrest, and induced cell death of cancer cells positive for MAN2A1-FER. FIG. 6A shows flow cytometry analyses of the immunofluorescence staining of antibody from hybridoma clone 2-3B-G8 (G8) or non-specific IgG. HEP3BMF(+) indicated HEP3B cells transformed with pCNDA4-MAN2A1-FER-FLAG/pCDNA6TO and induced with tetracycline, while HEP3BMF(−) was the uninduced control. FIG. 6B shows immunoblotting of protein extracts from HUH7 or HUH7ko cells treated with 10 ng/ml IgG or 2-3B-G8 (G8). Antibodies specific for FER, PDGFRA, phospho-PDGFRA (pY118/pY120), phospho-PDGFRA (pY1018), MEK, phospho-MEK(pS221), STAT3, phospho-STAT3(pY705), and GAPDH were applied. FIG. 6C shows that antibody from hybridoma clone 2-3B-G8 induced cell growth arrest in HUH7 cells but not HUH7ko cells. FIG. 6D shows scatter plot analyses of annexin V and propidium iodide staining of HUH7 or HUH7ko cells treated with 10 ng/ml IgG or 2-3B-G8 (G8). FIG. 6E shows that antibody from hybridoma clone 2-3B-G8 caused cell death of MAN2A1-FER positive cancers (HUH7, PC3, HepG2, LN-229, and SNU449) but had minimal impact on MAN2A1-FER negative HUH7ko cells.

FIGS. 7A-7F show the therapeutic effect of an antibody from hybridoma clone 2-3B-G8 on HEPG2 and HUH7 xenografted cancers. FIG. 7A shows that antibody from hybridoma clone 2-3B-G8 reduced the tumor burden of HEPG2 xenografted cancer in SCID mice. Mice were treated with 2-3B-G8-MMAE 14 days after HEPG2 xenografting. IgG-MMAE treatment is the placebo control. FIG. 7B shows that 2-3B-G8-MMAE eliminated the invasion from HEPG2 cancer. FIG. 7C shows that 2-3B-G8-MMAE reduced the mortality of the animals xenografted with HEPG2 cancer. FIG. 7D shows that 2-3B-G8-MMAE reduced the tumor burden of HUH7 but not HUH7ko cancer where MAN2A1-FER was disrupted. FIG. 7E shows that 2-3B-G8-MMAE reduced invasion of HUH7 but not HUH7ko cancers. FIG. 7F shows that 2-3B-G8-MMAE decreased mortality of HUH7 but not HUH7ko cancers.

FIGS. 8A-8D show MAN2A1-FER fusion expression and breakpoints in human cancer cell lines. FIG. 8A shows images of Tagman qRT-PCR of MAN2A1-FER and 0-actin on mRNA of human cancer cell lines. FIG. 8B shows images of Chromogram of Sanger's sequencing on the fusion juncture of MAN2A1-FER mRNA. FIG. 8C shows images of Tagman qPCR of MAN2A1-FER on genome DNA of human cancer cell lines. FIG. 8D shows images of Chromogram of Sanger's sequencing on the genome breakpoint juncture of MAN2A1-FER.

FIG. 9 shows the frequency of MAN2A1-FER expression in colon and breast cancer. The total number of cases of each type of cancer is indicated.

FIG. 10 shows validation of partial humanized 2-3B-G8 antibody specific for pY118/pY120. HRUH7 cells were stained without primary antibody (left), with non-specific IgG (middle); or with the partially humanized 2-3B-G8 antibody (right).

FIG. 11 shows validation of fully humanized 2-3B-G8 antibody specific for pY118/pY120. ELISA assays were performed on phospho-HisTAG-PDGFRAaa1-528 with non-specific IgG (IgG), or antibody specific for the N-terminus of human PDGFRA (anti-PDGFRA) or humanized G8 antibody specific for pY118/pY120 of human PDGFRA (H-G8). Triplicate experiments were performed. The optical densities and standard deviations of the ELISA assays are shown.

### DETAILED DESCRIPTION

The presently disclosed subject matter relates to antibodies and antigen-binding fragments that bind specifically to phosphorylated PDGFRA for use in treating cancer expressing MAN2A1-FER.

For purposes of clarity of disclosure and not by way of limitation, the detailed description is divided into the following subsections:
1. Definitions;
2. MAN2A1-FER Fusion Gene;
3. Antibodies and Antigen-Binding Fragments;
4. Diagnostic Methods and Methods of Treatment;
5. Pharmaceutical Compositions; and
6. Kits.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “fusion gene,” as used herein, refers to a nucleic acid or protein sequence which combines elements of the recited genes or their RNA transcripts in a manner not found in the wild type/normal nucleic acid or protein sequences. For example, but not by way of limitation, in a fusion gene in the form of genomic DNA, the relative positions of portions of the genomic sequences of the recited genes is altered relative to the wild type/normal sequence (for example, as reflected in the NCBI chromosomal positions or sequences set forth herein). In a fusion gene in the form of mRNA, portions of RNA transcripts arising from both component genes are present (not necessarily in the same register as the wild-type transcript and possibly including portions normally not present in the normal mature transcript). In non-limiting embodiments, such a portion of genomic DNA or mRNA can comprise at least about 10 consecutive nucleotides, or at least about 20 consecutive nucleotides, or at least about 30 consecutive nucleotides, or at least 40 consecutive nucleotides. In certain embodiments, such a portion of genomic DNA or mRNA can comprise up to about 10 consecutive nucleotides, up to about 50 consecutive nucleotides, up to about 100 consecutive nucleotides, up to about 200 consecutive nucleotides, up to about 300 consecutive nucleotides, up to about 400 consecutive nucleotides, up to about 500 consecutive nucleotides, up to about 600 consecutive nucleotides, up to about 700 consecutive nucleotides, up to about 800 consecutive nucleotides, up to about 900 consecutive nucleotides, up to about 1,000 consecutive nucleotides, up to about 1,500 consecutive nucleotides or up to about 2,000 consecutive nucleotides of the nucleotide sequence of a gene present in the fusion gene. In certain embodiments, such a portion of genomic DNA or mRNA can comprise no more than about 10 consecutive nucleotides, about 50 consecutive nucleotides, about 100 consecutive nucleotides, about 200 consecutive nucleotides, about 300 consecutive nucleotides, about 400 consecutive nucleotides, about 500 consecutive nucleotides, about 600 consecutive nucleotides, about 700 consecutive nucleotides, about 800 consecutive nucleotides, about 900 consecutive nucleotides, about 1,000 consecutive nucleotides, about 1,500 consecutive nucleotides or about 2,000 consecutive nucleotides of the nucleotide sequence of a gene present in the fusion gene. In certain embodiments, such a portion of genomic DNA or mRNA does not comprise the full wild type/normal nucleotide sequence of a gene present in the fusion gene. In a fusion gene in the form of a protein, portions of amino acid sequences arising from both component genes are present (not by way of limitation, at least about 5 consecutive amino acids or at least about 10 amino acids or at least about 20 amino acids or at least about 30 amino acids). In certain embodiments, such a portion of a fusion gene protein can comprise up to about 10 consecutive amino acids, up to about 20 consecutive amino acids, up to about 30 consecutive amino acids, up to about 40 consecutive amino acids, up to about 50 consecutive amino acids, up to about 60 consecutive amino acids, up to about 70 consecutive amino acids, up to about 80 consecutive amino acids, up to about 90 consecutive amino acids, up to about 100 consecutive amino acids, up to about 120 consecutive amino acids, up to about 140 consecutive amino acids, up to about 160 consecutive amino acids, up to about 180 consecutive amino acids, up to about 200 consecutive amino acids, up to about 220 consecutive amino acids, up to about 240 consecutive amino acids, up to about 260 consecutive amino acids, up to about 280 consecutive amino acids or up to about 300 consecutive amino acids of the amino acid sequence encoded by a gene present in the fusion gene. In certain embodiments, such a portion of a fusion gene protein can comprise no more than about 10 consecutive amino acids, about 20 consecutive amino acids, about 30 consecutive amino acids, about 40 consecutive amino acids, about 50 consecutive amino acids, about 60 consecutive amino acids, about 70 consecutive amino acids, about 80 consecutive amino acids, about 90 consecutive amino acids, about 100 consecutive amino acids, about 120 consecutive amino acids, about 140 consecutive amino acids, about 160 consecutive amino acids, about 180 consecutive amino acids, about 200 consecutive amino acids, about 220 consecutive amino acids, about 240 consecutive amino acids, about 260 consecutive amino acids, about 280 consecutive amino acids or about 300 consecutive amino acids of the amino acid sequence encoded by a gene present in the fusion gene. In certain embodiments, such a portion of a fusion gene protein does not comprise the full wild type/normal amino acid sequence encoded by a gene present in the fusion gene. In this paragraph, portions arising from both genes, transcripts or proteins do not refer to sequences which can happen to be identical in the wild type forms of both genes (that is to say, the portions are “unshared”). As such, a fusion gene represents, generally speaking, the splicing together or fusion of genomic elements not normally joined together. See WO 2015/103057 and WO 2016/01 1428, the contents of which are hereby incorporated by reference, for additional information regarding the disclosed fusion genes.

As used herein, the term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides, or sugars.

As used herein, the terms “pharmaceutically” or “pharmacologically acceptable” refer to molecular entities and compositions that do not produce adverse, allergic, or other untoward reactions when administered to an animal or a human.

As used herein, the term, “pharmaceutically acceptable carrier” includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

As used herein, the terms “effective amount” or “therapeutically effective amount” refer to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition. In certain embodiments, an effective amount can be formulated and/or administered in a plurality of doses, for example, as part of a dosing regimen.

As used herein, the term “treatment” refers to a therapeutic intervention that ameliorates a sign or symptom of a disease or pathological condition after it has begun to develop, or administering a compound or composition to a subject who does not exhibit signs of a disease or exhibits only early signs for the purpose of decreasing the risk of developing a pathology or condition, or diminishing the severity of a pathology or condition. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

As used herein, “preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

As used herein, the term “administered” or “administering” a drug, refers to any method of providing a compound or drug to a patient such that the compound or drug has its intended effect on the patient. For example, one method of administering is by an indirect mechanism using a medical device such as, but not limited to a catheter, spray gun, syringe etc. A second exemplary method of administering is by a direct mechanism such as, oral ingestion, transdermal patch, topical, inhalation, suppository etc.

The term “nanoparticle” as used herein, refers to any microscopic carrier to which a compound or drug can be attached. Nanoparticles generally refer to the general categories comprising liposomes, microparticles, microspheres, nanospheres, microcapsules, and nanocapsules. In certain embodiments, nanoparticles contemplated by this present disclosure are capable of formulations having controlled release properties.

As used herein, the term “PLGA” refers to mixtures of polymers or copolymers of lactic acid and glycolic acid. As used herein, lactide polymers are chemically equivalent to lactic acid polymer and glycolide polymers are chemically equivalent to glycolic acid polymers. In one embodiment, PLGA contemplates an alternating mixture of lactide and glycolide polymers, and is referred to as a poly(lactide-co-glycolide) polymer.

As used herein, the term “biocompatible” refers to any material does not elicit a substantial detrimental response in the host. There is always concern, when a foreign object is introduced into a living body, that the object will induce an immune reaction, such as an inflammatory response that will have negative effects on the host. In the context of this disclosed subject matter, biocompatibility is evaluated according to the application for which it was designed: for example; a bandage is regarded a biocompatible with the skin, whereas an implanted medical device is regarded as biocompatible with the internal tissues of the body. In certain embodiments, biocompatible materials include, but are not limited to, biodegradable and biostable materials.

As used herein, the term “biodegradable” refers to any material that can be acted upon biochemically by living cells or organisms, or processes thereof, including water, and broken down into lower molecular weight products such that the molecular structure has been altered.

As used herein, the term “polymer” refers to any unit-based chain of molecules. For example, such molecules can include but are not limited to gelatin, collagen, cellulose esters, dextran sulfate, pentosan polysulfate, chitin, saccharides, albumin, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

### 2. MAN2A1-FER Fusion Gene

The fusion gene MAN2A1-FER is a fusion between mannosidase, alpha, class 2A, member 1 (“MAN2A1”) and (fps/fes related) tyrosine kinase (“FER”). The human MAN2A1 gene is typically located on chromosome 5q21.3 and the human FER gene is typically located on chromosome 5q21. In certain embodiments, the MAN2A1 gene is the human gene having NCBI Gene ID NO: 4124, sequence chromosome 5; NC_000005.9 (109025156 . . . 109203429) or NC_000005.9 (109034137 . . . 109035578); and/or the FER gene is the human gene having NCBI Gene ID NO: 2241, sequence chromosome 5: NC_000005.9 (108083523 . . . 108523373).

### 3. Antibodies and Antigen-Binding Fragments

The presently disclosed subject matter relates to an antibody or antigen-binding fragment thereof that specifically binds to downstream targets of the MAN2A1-FER fusion gene. In certain embodiments, the antibody or antigen-binding fragment specifically binds to phosphorylated PDGFRA. The phrase “specifically binds” refers to binding of, for example, an antibody to an epitope or antigen or antigenic determinant in such a manner that binding can be displaced or competed with a second preparation of identical or similar epitope, antigen or antigenic determinant.

In certain embodiments, the antibody or antigen-binding fragment comprises a heavy chain protein sequence or a heavy chain variable domain sequence. In certain embodiments, the antibody or antigen-binding fragment comprises a light chain protein sequence or a light chain variable domain sequence. In certain embodiments, the antibody or antigen-binding fragment comprises a heavy chain protein sequence and a light chain protein sequence. In certain embodiments, the antibody or antigen-binding fragment comprises a heavy chain variable domain sequence and a light chain variable domain sequence. In certain embodiments, the antibody or antigen-binding fragment comprises one or more sequences listed in Table 1.

TABLE 1
Antibody or antigen-binding fragment sequences.

| Clone | Description | Sequence |
|---|---|---|
| 2-3B-G8 | light chain | ATGGATTTTCAAGTGCAGATTTTCAGCTTCCTGCTAA |
| | protein | TCAGTGCCTCAGTCATAATGTCCAGAGGAGAAAATG |
| | nucleotide | TTCTCACCCAGTCTCCAGCAATCATGTCTGCATCTC |
| | sequence | CAGGGGAAAAGGTCACCATGACCTGCAGTGCCAGC |
| | | TCAAGTGTAAGTTACATGCACTGGTACCAGCAGAAG |
| | | TCAAGCACCTCCCCCAAACTCTGGATTTATGACACA |
| | | TCCAAACTGGCTTCTGGAGTCCCTGCTCGCTTCAGT |
| | | GGCAGTGGGTCTGGGACCTCTTACTCTCTCACAATC |
| | | AGCAGCATGGAGGCTGAAGATGTTGCCACTTATTAC |
| | | TGTTTTCAGGGGAGTGGGTACCCACTCACGTTCGG |
| | | CTCGGGGACAAAGTTGGAAATAAAACGGGCTGATG |
| | | CTGCACCAACTGTATACATCTTCCCACCATCCAGTG |
| | | AGCAGTTAACATCTGGAGGTGCCTCAGTCGTGTGCT |
| | | TCTTGAACAACTTCTACCCCAAAGACATCAATGTCAA |
| | | GTGGAAGATTGATGGCAGTGAACGACAAAATGGCG |
| | | TCCTGAACAGTTGGACTGATCAGGACAGCAAAGACT |
| | | GCACCTACAGCATGAGCAGCACCCTCACGTTGACC |
| | | AAGGACGAGTATGAACGCCATAACAGCTATACCTGT |
| | | GAGGCCACTCACAAGACATCAACTTCACCCATTGTC |
| | | AAGAGCTTCAACAGGAATGAGTGT (SEQ ID NO.: 1) |
| 2-3B-G8 | light chain | MDFQVQIFSFLLISASVIMSRGENVLTQSPAIMSASPGE |
| | protein amino | KVTMTCSASSSVSYMHWYQQKSSTSPKLWIYDTSKLA |
| | acid sequence | SGVPARFSGSGSGTSYSLTISSMEAEDVATYYCFQGS |
| | | GYPLTFGSGTKLEIKRADAAPTVYIFPPSSEQLTSGGA |
| | | SVVCFLNNFYPKDINVKWKIDGSERQNGVLNSWTDQD |
| | | SKDCTYSMSSTLTLTKDEYERHNSYTCEATHKTSTSPI |
| | | VKSFNRNEC (SEQ ID NO.: 2) |
| 2-3B-G8 | light chain | ATGGATTTTCAAGTGCAGATTTTCAGCTTCCTGCTAA |
| | variable | TCAGTGCCTCAGTCATAATGTCCAGAGGAGAAAATG |
| | domain | TTCTCACCCAGTCTCCAGCAATCATGTCTGCATCTC |
| | nucleotide | CAGGGGAAAAGGTCACCATGACCTGCAGTGCCAGC |
| | sequence | TCAAGTGTAAGTTACATGCACTGGTACCAGCAGAAG |
| | | TCAAGCACCTCCCCCAAACTCTGGATTTATGACACA |
| | | TCCAAACTGGCTTCTGGAGTCCCTGCTCGCTTCAGT |
| | | GGCAGTGGGTCTGGGACCTCTTACTCTCTCACAATC |
| | | AGCAGCATGGAGGCTGAAGATGTTGCCACTTATTAC |
| | | TGTTTTCAGGGGAGTGGGTACCCACTCACGTTCGG |
| | | CTCGGGGACAAAGTTGGAAATAAAA (SEQ ID NO.: 3) |
| 2-3B-G8 | light chain | MDFQVQIFSFLLISASVIMSRGENVLTQSPAIMSASPGE |
| | variable | KVTMTCSASSSVSYMHWYQQKSSTSPKLWIYDTSKLA |
| | domain amino | SGVPARFSGSGSGTSYSLTISSMEAEDVATYYCFQGS |
| | acid sequence | GYPLTFGSGTKLEIK (SEQ ID NO.: 4) |
| 2-3B-G8 | heavy chain | ATGGAACTCTGGGGACCTGGCAGTACCGATGCAGC |
| | protein | CAAAACAACAGCCCCATCGGTCTATCCACTGGCCCC |
| | nucleotide | TGTGTGTGGAGATACAACTGGCTCCTCGGTGACTCT |
| | sequence | AGGATGCCTGGTCAAGGGTTATTTCCCTGAGCCAGT |
| | | GACCTTGACCTGGAACTCTGGATCCCTGTCCAGTG |
| | | GTGTGCACACCTTCCCAGCTGTCCTGCAGTCTGACC |
| | | TCTACACTCTGAGCAGCTCAGTGACTGTCCCCTCCA |
| | | GCACCTGGCCCAGCGAGACCGTCACCTGCAACGTT |
| | | GCCCACCCGGCCAGCAGCACCAAGGTGGACAAGAA |
| | | AATTGTGCCCAGGGATTGTGGTTGTAAGCCTTGCAT |
| | | ATGTACAGTCCCAGAAGTATCATCTGTCTTCATCTTC |
| | | CCCCCAAAGCCCAAGGATGTGCTCACCATTACTCTG |
| | | ACTCCTAAGGTCACGTGTGTTGTGGTAGACATCAGC |
| | | AAGGATGATCCCGAGGTCCAGTTCAGCTGGTTTGTA |
| | | GATGATGTGGAGGTGCACACAGCTCAGACGCAACC |
| | | CCGGGAGGAGCAGTTCAACAGCACTTTCCGCTCAG |
| | | TCAGTGAACTTCCCATCATGCACCAGGACTGGCTCA |
| | | ATGGCAAGGAGTTCAAATGCAGGGTCAACAGTGCA |
| | | GCTTTCCCTGCCCCCATCGAGAAAACCATCTCCAAA |
| | | ACCAAAGGCAGACCGAAGGCTCCACAGGTGTACAC |
| | | CATTCCACCTCCCAAGGAGCAGATGGCCAAGGATA |
| | | AAGTCAGTCTGACCTGCATGATAACAGACTTCTTCC |
| | | CTGAAGACATTACTGTGGAGTGGCAGTGGAATGGG |
| | | CAGCCAGCGGAGAACTACAAGAACACTCAGCCCAT |
| | | CATGGACACAGATGGCTCTTACTTCGTCTACAGCAA |
| | | GCTCAATGTGCAGAAGAGCAACTGGGAGGCAGGAA |
| | | ATACTTTCACCTGCTCTGTGTTACATGAGGGCCTGC |
| | | ACAACCACCATACTGAGAAGAGCCTCTCCCACTCTC |
| | | CTGGGCTGCAACTGGACGAGACCTGTGCTGAGGCC |
| | | CAGGACGGGGAGCTGGACGGGCTCTGGACGACCA |
| | | TCACCATCTTCATCAGCCTCTTCCTGCTCAGCGTGT |
| | | GCTACAGCGCTGCTGTCACACTCTTCAAGGTAAAGT |
| | | GGATCTTCTCCTCGGTGGTGGAGCTGAAGCAGACA |
| | | CTGGTTCCTGAATACAAGAACATGATTGGGCAAGCG |
| | | CCC (SEQ ID NO.: 5) |
| 2-3B-G8 | heavy chain | MELWGPGSTDAAKTTAPSVYPLAPVCGDTTGSSVTLG |
| | protein amino | CLVKGYFPEPVTLTWNSGSLSSGVHTFPAVLQSDLYT |
| | acid sequence | LSSSVTVPSSTWPSETVTCNVAHPASSTKVDKKIVPRD |
| | | CGCKPCICTVPEVSSVFIFPPKPKDVLTITLTPKVTCVV |
| | | VDISKDDPEVQFSWFVDDVEVHTAQTQPREEQFNSTF |
| | | RSVSELPIMHQDWLNGKEFKCRVNSAAFPAPIEKTISK |
| | | TKGRPKAPQVYTIPPPKEQMAKDKVSLTCMITDFFPED |
| | | ITVEWQWNGQPAENYKNTQPIMDTDGSYFVYSKLNV |
| | | QKSNWEAGNTFTCSVLHEGLHNHHTEKSLSHSPGLQ |
| | | LDETCAEAQDGELDGLWTTITIFISLFLLSVCYSAAVTLF |
| | | KVKWIFSSVVELKQTLVPEYKNMIGQAP (SEQ ID NO.: |
| | | 6) |
| 2-3B-G8 | heavy chain | ATGGAACTCTGGGGACCTGGCAGTACCGATGCA |
| | variable | (SEQ ID NO.: 7) |
| | domain | |
| | nucleotide | |
| | sequence | |
| 2-3B-G8 | heavy chain | MELWGPGSTDA (SEQ ID NO.: 8) |
| | variable | |
| | domain amino | |
| | acid sequence | |
| 2-3B-G8 | CDRL1 amino | SASSSVSY (SEQ ID NO.: 9) |
| | acid sequence | |
| 2-3B-G8 | CDRL2 amino | DTSKLAS (SEQ ID NO.: 10) |
| | acid sequence | |
| 2-3B-G8 | CDRL3 amino | FQGSGYPLT (SEQ ID NO.: 11) |
| | acid sequence | |
| 2-3B-G8 | light chain | MDFQVQIFSFLLISASVIMSRGENVLTQSPAIMSASPGE |
| | protein | KVTMTCSASSSVSYMHWYQQKSSTSPKLWIYDTSKLA |
| | humanized | SGVPARFSGSGSGTSYSLTISSMEAEDVATYYCFQGS |
| | amino acid | GYPLTFGSGTKLEIKRTVAAPSVFIFPPSDEQLKSGTAS |
| | sequence | VVCLLNNFYPREAKVQWKVDNALQSGNSQESVTEQD |
| | | SKDSTYSLSSTLTLSKADYEKHKVYACEVTHQGLSSPV |
| | | TKSFNRGEC (SEQ ID NO.: 12) |
| 2-3B-G8 | heavy chain | MELWGPGSTDAASTKGPSVFPLAPSSKSTSGGTAAL |
| | protein | GCLVKDYFPEPVTVSWNSGALTSGVHTFPAVLQSSGL |
| | humanized | YSLSSVVTVPSSSLGTQTYICNVNHKPSNTKVDKKVEP |
| | amino acid | KSCDKTHTCPPCPAPELLGGPSVFLFPPKPKDTLMISR |
| | sequence | TPEVTCVVVDVSHEDPEVKFNWYVDGVEVHNAKTKP |
| | | REEQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK (SEQ ID NO.: 13) |
| 2-3B-G8 | light chain | MQVVLTQSPGIMSASPGEKVTITCSASSSVSYMYWFQ |
| | protein | QKPGTSPKLWIYDTSKLASGVPARFRGSGSGTSYSLTI |
| | humanized | SRMEAEDAATYYCFQGSGYPLTFGGGTKLEIKRTVAA |
| | amino acid | PSVFIFPPSDEQLKSGTASVVCLLNNFYPREAKVQWK |
| | sequence | VDNALQSGNSQESVTEQDSKDSTYSLSSTLTLSKADY |
| | | EKHKVYACEVTHQGLSSPVTKSFNRGEC (SEQ ID |
| | | NO.: 14) |
| 2-3B-G8 | light chain | MQVVLTQSPGIMSASPGEKVTITCSASSSVSYMYWFQ |
| | variable | QKPGTSPKLWIYDTSKLASGVPARFRGSGSGTSYSLTI |
| | domain | SRMEAEDAATYYCFQGSGYPLTFGGGTKLEIK (SEQ |
| | humanized | ID NO.: 15) |
| | amino acid | |
| | sequence | |
| 2-3B-G8 | CDRL1 | SASSSVSY (SEQ ID NO.: 16) |
| | humanized | |
| | amino acid | |
| | sequence | |
| 2-3B-G8 | CDRL2 | DTSKLAS (SEQ ID NO.: 17) |
| | humanized | |
| | amino acid | |
| | sequence | |
| 2-3B-G8 | CDRL3 | FQGSGYPLT (SEQ ID NO.: 18) |
| | humanized | |
| | amino acid | |
| | sequence | |
| 1-4B-B10 | light chain | ATGGATTTTCAAGTGCAGATTTTCAGCTTCCTGCTAA |
| | protein | TCAGTGCCTCAGTCATAATGTCCAGAGGAGAAAATG |
| | nucleotide | TTCTCACCCAGTCTCCAGCAATCATGTCTGCATCTC |
| | sequence | CAGGGGAAAAGGTCACCATGACCTGCAGTGCCAGC |
| | | TCAAGTGTAAGTTACATGCACTGGTACCAGCAGAAG |
| | | TCAAGCACCTCCCCCAAACTCTGGATTTATGACACA |
| | | TCCAAACTGGCTTCTGGAGTCCCAGGTCGCTTCAGT |
| | | GGCAGTGGGTCTGGAAACTCTTACTCTCTCACGATC |
| | | AGCAGCATGGAGGCTGAAGATGTTGCCACTTATTAC |
| | | TGTTTTCAGGGGAGTGGGTACCCACTCACGTTCGG |
| | | CTCGGGGACAAAGTTGGAAATAAAACGGGCTGATG |
| | | CTGCACCAACTGTATCCATCTTCCCACCATCCAGTG |
| | | AGCAGTTAACATCTGGAGGTGCCTCAGTCGTGTGCT |
| | | TCTTGAACAACTTCTACCCCAAAGACATCAATGTCAA |
| | | GTGGAAGATTGATGGCAGTGAACGACAAAATGGCG |
| | | TCCTGAACAGTTGGACTGATCAGGACAGCAAAGACA |
| | | GCACCTACAGCATGAGCAGCACCCTCACGATGACC |
| | | AAGGACGAGTATGAACGACATAACAGCTATACCTGT |
| | | GAGGCCACTCACAAGACATCAACTTCACCCATTGTC |
| | | AAGAGCTTCAACAGGAATGAGTGT (SEQ ID NO.: 19) |
| 1-4B-B10 | light chain | MDFQVQIFSFLLISASVIMSRGENVLTQSPAIMSASPGE |
| | protein amino | KVTMTCSASSSVSYMHWYQQKSSTSPKLWIYDTSKLA |
| | acid sequence | SGVPGRFSGSGSGNSYSLTISSMEAEDVATYYCFQGS |
| | | GYPLTFGSGTKLEIKRADAAPTVSIFPPSSEQLTSGGA |
| | | SVVCFLNNFYPKDINVKWKIDGSERQNGVLNSWTDQD |
| | | SKDSTYSMSSTLTMTKDEYERHNSYTCEATHKTSTSPI |
| | | VKSFNRNEC (SEQ ID NO.: 20) |
| 1-4B-B10 | light chain | ATGGATTTTCAAGTGCAGATTTTCAGCTTCCTGCTAA |
| | variable | TCAGTGCCTCAGTCATAATGTCCAGAGGAGAAAATG |
| | domain | TTCTCACCCAGTCTCCAGCAATCATGTCTGCATCTC |
| | nucleotide | CAGGGGAAAAGGTCACCATGACCTGCAGTGCCAGC |
| | sequence | TCAAGTGTAAGTTACATGCACTGGTACCAGCAGAAG |
| | | TCAAGCACCTCCCCCAAACTCTGGATTTATGACACA |
| | | TCCAAACTGGCTTCTGGAGTCCCAGGTCGCTTCAGT |
| | | GGCAGTGGGTCTGGAAACTCTTACTCTCTCACGATC |
| | | AGCAGCATGGAGGCTGAAGATGTTGCCACTTATTAC |
| | | TGTTTTCAGGGGAGTGGGTACCCACTCACGTTCGG |
| | | CTCGGGGACAAAGTTGGAAATAAAA (SEQ ID NO.: |
| | | 21) |
| 1-4B-B10 | light chain | MDFQVQIFSFLLISASVIMSRGENVLTQSPAIMSASPGE |
| | variable | KVTMTCSASSSVSYMHWYQQKSSTSPKLWIYDTSKLA |
| | domain amino | SGVPGRFSGSGSGNSYSLTISSMEAEDVATYYCFQGS |
| | acid sequence | GYPLTFGSGTKLEIK (SEQ ID NO.: 22) |
| 1-4B-B10 | heavy chain | ATGTGCCGACTTCAATTTGTGCTTCAGTGTTGTCCA |
| | protein | GATTGTGTGCAGCCATATGGCCCAGCCAAAACGAC |
| | nucleotide | ACCCCCATCTGTCTATCCACTGGCCCCTGGATCTGC |
| | sequence | TGCCCAAACTAACTCCATGGTGACCCTGGGATGCCT |
| | | GGTCAAGGGCTATTTCCCTGAGCCAGTGACAGTGA |
| | | CCTGGAACTCTGGATCCCTGTCCAGCGGTGTGCAC |
| | | ACCTTCCCAGCTGTCCTGCAGTCTGACCTCTACACT |
| | | CTGAGCAGCTCAGTGACTGTCCCCTCCAGCACCTG |
| | | GCCCAGCGAGACCGTCACCTGCAACGTTGCCCACC |
| | | CGGCCAGCAGCACCAAGGTGGACAAGAAAATTGTG |
| | | CCCAGGGATTGTGGTTGTAAGCCTTGCATATGTACA |
| | | GTCCCAGAAGTATCATCTGTCTTCATCTTCCCCCCA |
| | | AAGCCCAAGGATGTGCTCACCATTACTCTGACTCCT |
| | | AAGGTCACGTGTGTTGTGGTAGACATCAGCAAGGAT |
| | | GATCCCGAGGTCCAGTTCAGCTGGTTTGTAGATGAT |
| | | GTGGAGGTGCACACAGCTCAGACGCAACCCCGGGA |
| | | GGAGCAGTTCAACAGCACTTTCCGCTCAGTCAGTGA |
| | | ACTTCCCATCATGCACCAGGACTGGCTCAATGGCAA |
| | | GGAGTTCAAATGCAGGGTCAACAGTGCAGCTTTCCC |
| | | TGCCCCCATCGAGAAAACCATCTCCAAAACCAAAGG |
| | | CAGACCGAAGGCTCCACAGGTGTACACCATTCCAC |
| | | CTCCCAAGGAGCAGATGGCCAAGGATAAAGTCAGT |
| | | CTGACCTGCATGATAACAGACTTCTTCCCTGAAGAC |
| | | ATTACTGTGGAGTGGCAGTGGAATGGGCAGCCAGC |
| | | GGAGAACTACAAGAACACTCAGCCCATCATGGACAC |
| | | AGATGGCTCTTACTTCGTCTACAGCAAGCTCAATGT |
| | | GCAGAAGAGCAACTGGGAGGCAGGAAATACTTTCA |
| | | CCTGCTCTGTGTTACATGAGGGCCTGCACAACCACC |
| | | ATACTGAGAAGAGCCTCTCCCACTCTCCTGGGCTGC |
| | | AACTGGACGAGACCTGTGCTGAGGCCCAGGACGGG |
| | | GAGCTGGACGGGCTCTGGACGACCATCACCATCTT |
| | | CATCAGCCTCTTCCTGCTCAGCGTGTGCTACAGCGC |
| | | TGCTGTCACACTCTTCAAGGTAAAGTGGATCTTCTC |
| | | CTCGGTGGTGGAGCTGAAGCAGACACTGGTTCCTG |
| | | AATACAAGAACATGATTGGGCAAGCGCCC (SEQ ID |
| | | NO.: 23) |
| 1-4B-B10 | heavy chain | MCRLQFVLQCCPDCVQPYGPAKTTPPSVYPLAPGSA |
| | protein amino | AQTNSMVTLGCLVKGYFPEPVTVTWNSGSLSSGVHTF |
| | acid sequence | PAVLQSDLYTLSSSVTVPSSTWPSETVTCNVAHPASS |
| | | TKVDKKIVPRDCGCKPCICTVPEVSSVFIFPPKPKDVLT |
| | | ITLTPKVTCVVVDISKDDPEVQFSWFVDDVEVHTAQTQ |
| | | PREEQFNSTFRSVSELPIMHQDWLNGKEFKCRVNSAA |
| | | FPAPIEKTISKTKGRPKAPQVYTIPPPKEQMAKDKVSLT |
| | | CMITDFFPEDITVEWQWNGQPAENYKNTQPIMDTDGS |
| | | YFVYSKLNVQKSNWEAGNTFTCSVLHEGLHNHHTEKS |
| | | LSHSPGLQLDETCAEAQDGELDGLWTTITIFISLFLLSV |
| | | CYSAAVTLFKVKWIFSSVVELKQTLVPEYKNMIGQAP |
| | | (SEQ ID NO.: 24) |
| 1-4B-B10 | CDRL1 amino | SSVSY (SEQ ID NO.: 25) |
| | acid sequence | |
| 1-4B-B10 | CDRL2 amino | DTS (SEQ ID NO.: 26) |
| | acid sequence | |
| 1-4B-B10 | CDRL3 amino | FQGSGYPLT (SEQ ID NO.: 27) |
| | acid sequence | |
| 1-4B-B10 | light chain | MDFQVQIFSFLLISASVIMSRGENVLTQSPAIMSASPGE |
| | protein | KVTMTCSASSSVSYMHWYQQKSSTSPKLWIYDTSKLA |
| | humanized | SGVPGRFSGSGSGNSYSLTISSMEAEDVATYYCFQGS |
| | amino acid | GYPLTFGSGTKLEIKRTVAAPSVFIFPPSDEQLKSGTAS |
| | sequence | VVCLLNNFYPREAKVQWKVDNALQSGNSQESVTEQD |
| | | SKDSTYSLSSTLTLSKADYEKHKVYACEVTHQGLSSPV |
| | | TKSFNRGEC (SEQ ID NO.: 28) |
| 1-3C-F11 | light chain | ATGAGAAGAGAAGTAATTAGCCAGGGAACAAAATTC |
| | protein | AAATACACAATGGATTTTCTGGTGCAGATTTTCAGCT |
| | nucleotide | TCTTGCTAATCAGTGCCTCAGTTGCAATGTCCAGAG |
| | sequence | GAGAAAATGTGCTCACCCAGTCTCCAGCAATCATGT |
| | | CTGCATCTCCAGGGGAAAAGGTCACCATGACCTGC |
| | | AGGGCCAGCTCAAGTGTAAGTTCCAGTTACTTGCAC |
| | | TGGTACCAGCAGAAGTCAGGTGCCTCCCCCAAACT |
| | | CTGGATTTATAGCACATCCAACTTGGCTTCTGGAGT |
| | | CCCTGCTCGCTTCAGTGGCAGTGGGTCTGGGACCT |
| | | CTTACTCTCTCACAATCAGCAGTGTGGAGGCTGAAG |
| | | ATGCTGCCACTTATTACTGCCAGCAGTACAGTGGTT |
| | | ACCCACTAACGTTCGGAGGGGGGACCAAGCTGGAA |
| | | ATAAAACGGGCTGATGCTGCACCAACTGTATCCATC |
| | | TTCCCACCATCCAGTGAGCAGTTAACATCTGGAGGT |
| | | GCCTCAGTCGTGTGCTTCTTGAACAACTTCTACCCC |
| | | AAAGACATCAATGTCAAGTGGAAGATTGATGGCAGT |
| | | GAACGACAAAATGGCGTCCTGAACAGTTGGACTGAT |
| | | CAGGACAGCAAAGACAGCACCTACAGCATGAGCAG |
| | | CACCCTCACGTTGACCAAGGACGAGTATGAACGAC |
| | | ATAACAGCTATACCTGTGAGGCCACTCACAAGACAT |
| | | CAACTTCACCCATTGTCAAGAGCTTCAACAGGAATG |
| | | AGTGT (SEQ ID NO.: 29) |
| 1-3C-F11 | light chain | MRREVISQGTKFKYTMDFLVQIFSFLLISASVAMSRGE |
| | protein amino | NVLTQSPAIMSASPGEKVTMTCRASSSVSSSYLHWYQ |
| | acid sequence | QKSGASPKLWIYSTSNLASGVPARFSGSGSGTSYSLTI |
| | | SSVEAEDAATYYCQQYSGYPLTFGGGTKLEIKRADAA |
| | | PTVSIFPPSSEQLTSGGASVVCFLNNFYPKDINVKWKI |
| | | DGSERQNGVLNSWTDQDSKDSTYSMSSTLTLTKDEY |
| | | ERHNSYTCEATHKTSTSPIVKSFNRNEC (SEQ ID NO.: |
| | | 30) |
| 1-3C-F11 | light chain | ATGAGAAGAGAAGTAATTAGCCAGGGAACAAAATTC |
| | variable | AAATACACAATGGATTTTCTGGTGCAGATTTTCAGCT |
| | domain | TCTTGCTAATCAGTGCCTCAGTTGCAATGTCCAGAG |
| | nucleotide | GAGAAAATGTGCTCACCCAGTCTCCAGCAATCATGT |
| | sequence | CTGCATCTCCAGGGGAAAAGGTCACCATGACCTGC |
| | | AGGGCCAGCTCAAGTGTAAGTTCCAGTTACTTGCAC |
| | | TGGTACCAGCAGAAGTCAGGTGCCTCCCCCAAACT |
| | | CTGGATTTATAGCACATCCAACTTGGCTTCTGGAGT |
| | | CCCTGCTCGCTTCAGTGGCAGTGGGTCTGGGACCT |
| | | CTTACTCTCTCACAATCAGCAGTGTGGAGGCTGAAG |
| | | ATGCTGCCACTTATTACTGCCAGCAGTACAGTGGTT |
| | | ACCCACTAACGTTCGGAGGGGGGACCAAGCTGGAA |
| | | ATAAAA (SEQ ID NO.: 31) |
| 1-3C-F11 | light chain | MRREVISQGTKFKYTMDFLVQIFSFLLISASVAMSRGE |
| | variable | NVLTQSPAIMSASPGEKVTMTCRASSSVSSSYLHWYQ |
| | domain amino | QKSGASPKLWIYSTSNLASGVPARFSGSGSGTSYSLTI |
| | acid sequence | SSVEAEDAATYYCQQYSGYPLTFGGGTKLEIK (SEQ |
| | | ID NO.: 32) |
| 1-3C-F11 | heavy chain | ATGGTGACCCTGGGATGCCTGGTCAAGGGCTATTT |
| | protein | CCCTGAGCCAGTGACAGTGACCTGGAACTCTGGAT |
| | nucleotide | CCCTGTCCAGCGGTGTGCACACCTTCCCAGCTGTC |
| | sequence | CTGCAGTCTGACCTCTACACTCTGAGCAGCTCAGTG |
| | | ACTGTCCCCTCCAGCACCTGGCCCAGCGAGACCGT |
| | | CACCTGCAACGTTGCCCACCCGGCCAGCAGCACCA |
| | | AGGTGGACAAGAAAATTGTGCCCAGGGATTGTGGTT |
| | | GTAAGCCTTGCATATGTACAGTCCCAGAAGTATCAT |
| | | CTGTCTTCATCTTCCCCCCAAAGCCCAAGGATGTGC |
| | | TCACCATTACTCTGACTCCTAAGGTCACGTGTGTTG |
| | | TGGTAGACATCAGCAAGGATGATCCCGAGGTCCAG |
| | | TTCAGCTGGTTTGTAGATGATGTGGAGGTGCACACA |
| | | GCTCAGACGCAACCCCGGGAGGAGCAGTTCAACAG |
| | | CACTTTCCGCTCAGTCAGTGAACTTCCCATCATGCA |
| | | CCAGGACTGGCTCAATGGCAAGGAGTTCAAATGCA |
| | | GGGTCAACAGTGCAGCTTTCCCTGCCCCCATCGAG |
| | | AAAACCATCTCCAAAACCAAAGGCAGACCGAAGGCT |
| | | CCACAGGTGTACACCATTCCACCTCCCAAGGAGCA |
| | | GATGGCCAAGGATAAAGTCAGTCTGACCTGCATGAT |
| | | AACAGACTTCTTCCCTGAAGACATTACTGTGGAGTG |
| | | GCAGTGGAATGGGCAGCCAGCGGAGAACTACAAGA |
| | | ACACTCAGCCCATCATGGACACAGATGGCTCTTACT |
| | | TCGTCTACAGCAAGCTCAATGTGCAGAAGAGCAACT |
| | | GGGAGGCAGGAAATACTTTCACCTGCTCTGTGTTAC |
| | | ATGAGGGCCTGCACAACCACCATACTGAGAAGAGC |
| | | CTCTCCCACTCTCCTGGGCTGCAACTGGACGAGAC |
| | | CTGTGCTGAGGCCCAGGACGGGGAGCTGGACGGG |
| | | CTCTGGACGACCATCACCATCTTCATCAGCCTCTTC |
| | | CTGCTCAGCGTGTGCTACAGCGCCTCTGTCACACTC |
| | | TTCAAGGTAAAGTGGATCTTCTCCTCGGTGGTGGAG |
| | | CTGAAGCAGACACTGGTTCCTGAATACAAGAACATG |
| | | ATTGGGCAAGCGCCC (SEQ ID NO.: 33) |
| 1-3C-F11 | heavy chain | MVTLGCLVKGYFPEPVTVTWNSGSLSSGVHTFPAVLQ |
| | protein amino | SDLYTLSSSVTVPSSTWPSETVTCNVAHPASSTKVDK |
| | acid sequence | KIVPRDCGCKPCICTVPEVSSVFIFPPKPKDVLTITLTPK |
| | | VTCVVVDISKDDPEVQFSWFVDDVEVHTAQTQPREEQ |
| | | FNSTFRSVSELPIMHQDWLNGKEFKCRVNSAAFPAPIE |
| | | KTISKTKGRPKAPQVYTIPPPKEQMAKDKVSLTCMITD |
| | | FFPEDITVEWQWNGQPAENYKNTQPIMDTDGSYFVYS |
| | | KLNVQKSNWEAGNTFTCSVLHEGLHNHHTEKSLSHSP |
| | | GLQLDETCAEAQDGELDGLWTTITIFISLFLLSVCYSAS |
| | | VTLFKVKWIFSSVVELKQTLVPEYKNMIGQAP (SEQ ID |
| | | NO.: 34) |
| 1-3C-F11 | CDRL1 amino | SSVSSSY (SEQ ID NO.: 35) |
| | acid sequence | |
| 1-3C-F11 | CDRL2 amino | STS (SEQ ID NO.: 36) |
| | acid sequence | |
| 1-3C-F11 | CDRL3 amino | QQYSGYPLT (SEQ ID NO.: 37) |
| | acid sequence | |
| 1-3C-F11 | light chain | MRREVISQGTKFKYTMDFLVQIFSFLLISASVAMSRGE |
| | protein | NVLTQSPAIMSASPGEKVTMTCRASSSVSSSYLHWYQ |
| | humanized | QKSGASPKLWIYSTSNLASGVPARFSGSGSGTSYSLTI |
| | amino acid | SSVEAEDAATYYCQQYSGYPLTFGGGTKLEIKRTVAA |
| | sequence | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC (SEQ ID |
| | | NO.: 38) |

In certain embodiments, the antibody or antigen-binding fragment comprises a light chain protein sequence comprising a nucleic acid sequences that is at least 70%, 75%, 80%, 85%, 90%, 95%, or 100% identical to SEQ ID NO.: 1. In certain embodiments, the antibody or antigen-binding fragment comprises a light chain protein sequence comprising an amino acid sequence that is at least 70%, 75%, 80%, 85%, 90%, 95%, or 100% identical to SEQ ID NO.: 2. In certain embodiments, the antibody or antigen-binding fragment comprises a light chain variable domain sequence comprising a nucleic acid sequence that is at least 70%, 75%, 80%, 85%, 90%, 95%, or 100% identical to SEQ ID NO.: 3. In certain embodiments, the antibody or antigen-binding fragment comprises a light chain variable domain sequence comprising an amino acid sequence that is at least 70%, 75%, 80%, 85%, 90%, 95%, or 100% identical to SEQ ID NO.: 4.

In certain embodiments, the antibody or antigen-binding fragment comprises a heavy chain protein sequence comprising a nucleic acid sequence that is at least 70%, 75%, 80%, 85%, 90%, 95%, or 100% identical to SEQ ID NO.: 5. In certain embodiments, the antibody or antigen-binding fragment comprises a heavy chain protein sequence comprising an amino acid sequence that is at least 70%, 75%, 80%, 85%, 90%, 95%, or 100% identical to SEQ ID NO.: 6. In certain embodiments, the antibody or antigen-binding fragment comprises a heavy chain variable domain sequence comprising a nucleic acid sequence that is at least 70%, 75%, 80%, 85%, 90%, 95%, or 100% identical to SEQ ID NO.: 7. In certain embodiments, the antibody or antigen-binding fragment comprises a heavy chain variable domain sequence comprising an amino acid sequence that is at least 70%, 75%, 80%, 85%, 90%, 95%, or 100% identical to SEQ ID NO.: 8.

In certain embodiments, the antibody or antigen-binding fragment comprises light chain complementarity determining regions (CDRs) having amino acid sequences that are at least 70%, 75%, 80%, 85%, 90%, 95%, or 100% identical to SEQ ID NO.: 9, SEQ ID NO.: 10, and SEQ ID NO.: 11. In certain embodiments, the antibody or antigen-binding fragment comprises a light chain CDR-1 (CDRL1) having an amino acid sequence that is at least 70%, 75%, 80%, 85%, 90%, 95%, or 100% identical to SEQ ID NO.: 9. In certain embodiments, the antibody or antigen-binding fragment comprises a light chain CDR-2 (CDRL2) having an amino acid sequence that is at least 70%, 75%, 80%, 85%, 90%, 95%, or 100% identical to SEQ ID NO.: 10. In certain embodiments, the antibody or antigen-binding fragment comprises a light chain CDR-3 (CDRL3) having an amino acid sequence that is at least 70%, 75%, 80%, 85%, 90%, 95%, or 100% identical to SEQ ID NO.: 11. In certain embodiments, the antibody or antigen-binding fragment comprises CDRL1, CDRL2, and CDRL3 having amino acid sequences that are at least 70%, 75%, 80%, 85%, 90%, 95%, or 100% identical to SEQ ID NO.: 9, SEQ ID NO.: 10, and SEQ ID NO.: 11, respectively.

In certain embodiments, the antibody or antigen-binding fragment comprises humanized sequences. In certain embodiments, the antibody or antigen-binding fragment comprises a humanized light chain protein sequence comprising amino acid sequences that are at least 70%, 75%, 80%, 85%, 90%, 95%, or 100% identical to the amino acid sequences selected from the group consisting of SEQ ID NO.: 12 and SEQ ID NO.: 14. In certain embodiments, the antibody or antigen-binding fragment comprises a humanized light chain variable domain sequence comprising an amino acid sequence that is at least 70%, 75%, 80%, 85%, 90%, 95%, or 100% identical to SEQ ID NO.: 15. In certain embodiments, the antibody or antigen-binding fragment comprises a humanized heavy chain protein sequence comprising an amino acid sequence that is at least 70%, 75%, 80%, 85%, 90%, 95%, or 100% identical to SEQ ID NO.: 13.

In certain embodiments, the antibody or antigen-binding fragment comprises humanized light chain CDRs having amino acid sequences that are at least 70%, 75%, 80%, 85%, 90%, 95%, or 100% identical to SEQ ID NO.: 16, SEQ ID NO.: 17, and SEQ ID NO.: 18. In certain embodiments, the antibody or antigen-binding fragment comprises a humanized light chain CDRL1 having an amino acid sequence that is at least 70%, 75%, 80%, 85%, 90%, 95%, or 100% identical to SEQ ID NO.: 16. In certain embodiments, the antibody or antigen-binding fragment comprises a humanized light chain CDRL2 having an amino acid sequence that is at least 70%, 75%, 80%, 85%, 90%, 95%, or 100% identical to SEQ ID NO.: 17. In certain embodiments, the antibody or antigen-binding fragment comprises a humanized light chain CDRL3 having an amino acid sequence that is at least 70%, 75%, 80%, 85%, 90%, 95%, or 100% identical to SEQ ID NO.: 18. In certain embodiments, the antibody or antigen-binding fragment comprises humanized CDRL1, CDRL2, and CDRL3 having amino acid sequences that are at least 70%, 75%, 80%, 85%, 90%, 95%, or 100% identical to SEQ ID NO.: 16, SEQ ID NO.: 17, and SEQ ID NO.: 18, respectively.

In certain embodiments, the antibody or antigen-binding fragment comprises heavy/light chain protein sequences comprising amino acid sequences that are at least 70%, 75%, 80%, 85%, 90%, 95%, or 100% identical to the amino acid sequences selected from the group consisting of SEQ ID NO.: 6/SEQ ID NO.: 2, SEQ ID NO.: 6/SEQ ID NO.: 12, SEQ ID NO.: 6/SEQ ID NO.: 14, SEQ ID NO.: 13/SEQ ID NO.: 2, SEQ ID NO.: 13/SEQ ID NO.: 12, and SEQ ID NO.: 13/SEQ ID NO.: 14. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 6/SEQ ID NO.: 2. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 13/SEQ ID NO.: 12. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 13/SEQ ID NO.: 14.

In certain embodiments, the antibody or antigen-binding fragment comprises heavy/light chain variable domain sequences comprising amino acid sequences that are at least 70%, 75%, 80%, 85%, 90%, 95%, or 100% identical to the amino acid sequences selected from the group consisting of SEQ ID NO.: 8/SEQ ID NO.: 4 and SEQ ID NO.: 8/SEQ ID NO.: 15. In certain embodiments, the antibody or antigen-binding fragment comprises heavy/light chain variable domain sequences comprising amino acid sequences that are at least 70%, 75%, 80%, 85%, 90%, 95%, or 100% identical to SEQ ID NO.: 8/SEQ ID NO.: 4. In certain embodiments, the antibody or antigen-binding fragment comprises heavy/light chain variable domain sequences comprising amino acid sequences that are at least 70%, 75%, 80%, 85%, 90%, 95%, or 100% identical to SEQ ID NO.: 8/SEQ ID NO.: 15.

In certain embodiments, the antibody or antigen-binding fragment comprises a light chain protein sequence comprising a nucleic acid sequences that is at least 70%, 75%, 80%, 85%, 90%, 95%, or 100% identical to SEQ ID NO.: 19. In certain embodiments, the antibody or antigen-binding fragment comprises a light chain protein sequence comprising an amino acid sequence that is at least 70%, 75%, 80%, 85%, 90%, 95%, or 100% identical to SEQ ID NO.: 20. In certain embodiments, the antibody or antigen-binding fragment comprises a light chain variable domain sequence comprising a nucleic acid sequence that is at least 70%, 75%, 80%, 85%, 90%, 95%, or 100% identical to SEQ ID NO.: 21. In certain embodiments, the antibody or antigen-binding fragment comprises a light chain variable domain sequence comprising an amino acid sequence that is at least 70%, 75%, 80%, 85%, 90%, 95%, or 100% identical to SEQ ID NO.: 22.

In certain embodiments, the antibody or antigen-binding fragment comprises a heavy chain protein sequence comprising a nucleic acid sequence that is at least 70%, 75%, 80%, 85%, 90%, 95%, or 100% identical to SEQ ID NO.: 23. In certain embodiments, the antibody or antigen-binding fragment comprises a heavy chain protein sequence comprising an amino acid sequence that is at least 70%, 75%, 80%, 85%, 90%, 95%, or 100% identical to SEQ ID NO.: 24.

In certain embodiments, the antibody or antigen-binding fragment comprises light chain complementarity determining regions (CDRs) having amino acid sequences that are at least 70%, 75%, 80%, 85%, 90%, 95%, or 100% identical to SEQ ID NO.: 25, SEQ ID NO.: 26, and SEQ ID NO.: 27. In certain embodiments, the antibody or antigen-binding fragment comprises a light chain CDRL1 having an amino acid sequence that is at least 70%, 75%, 80%, 85%, 90%, 95%, or 100% identical to SEQ ID NO.: 25. In certain embodiments, the antibody or antigen-binding fragment comprises a light chain CDRL2 having an amino acid sequence that is at least 70%, 75%, 80%, 85%, 90%, 95%, or 100% identical to SEQ ID NO.: 26. In certain embodiments, the antibody or antigen-binding fragment comprises a light chain CDRL3 having an amino acid sequence that is at least 70%, 75%, 80%, 85%, 90%, 95%, or 100% identical to SEQ ID NO.: 27. In certain embodiments, the antibody or antigen-binding fragment comprises CDRL1, CDRL2, and CDRL3 having amino acid sequences that are at least 70%, 75%, 80%, 85%, 90%, 95%, or 100% identical to SEQ ID NO.: 25, SEQ ID NO.: 26, and SEQ ID NO.: 27, respectively.

In certain embodiments, the antibody or antigen-binding fragment comprises humanized sequences. In certain embodiments, the antibody or antigen-binding fragment comprises a humanized light chain protein sequence comprising an amino acid sequences that are at least 70%, 75%, 80%, 85%, 90%, 95%, or 100% identical to SEQ ID NO.: 28.

In certain embodiments, the antibody or antigen-binding fragment comprises heavy/light chain protein sequences comprising amino acid sequences that are at least 70%, 75%, 80%, 85%, 90%, 95%, or 100% identical to the amino acid sequences selected from the group consisting of SEQ ID NO.: 24/SEQ ID NO.: 20 and SEQ ID NO.: 24/SEQ ID NO.: 28. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 24/SEQ ID NO.: 20. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 24/SEQ ID NO.: 28.

In certain embodiments, the antibody or antigen-binding fragment comprises a light chain protein sequence comprising a nucleic acid sequences that is at least 70%, 75%, 80%, 85%, 90%, 95%, or 100% identical to SEQ ID NO.: 29. In certain embodiments, the antibody or antigen-binding fragment comprises a light chain protein sequence comprising an amino acid sequence that is at least 70%, 75%, 80%, 85%, 90%, 95%, or 100% identical to SEQ ID NO.: 30. In certain embodiments, the antibody or antigen-binding fragment comprises a light chain variable domain sequence comprising a nucleic acid sequence that is at least 70%, 75%, 80%, 85%, 90%, 95%, or 100% identical to SEQ ID NO.: 31. In certain embodiments, the antibody or antigen-binding fragment comprises a light chain variable domain sequence comprising an amino acid sequence that is at least 70%, 75%, 80%, 85%, 90%, 95%, or 100% identical to SEQ ID NO.: 32.

In certain embodiments, the antibody or antigen-binding fragment comprises a heavy chain protein sequence comprising a nucleic acid sequence that is at least 70%, 75%, 80%, 85%, 90%, 95%, or 100% identical to SEQ ID NO.: 33. In certain embodiments, the antibody or antigen-binding fragment comprises a heavy chain protein sequence comprising an amino acid sequence that is at least 70%, 75%, 80%, 85%, 90%, 95%, or 100% identical to SEQ ID NO.: 34.

In certain embodiments, the antibody or antigen-binding fragment comprises light chain complementarity determining regions (CDRs) having amino acid sequences that are at least 70%, 75%, 80%, 85%, 90%, 95%, or 100% identical to SEQ ID NO.: 35, SEQ ID NO.: 36, and SEQ ID NO.: 37. In certain embodiments, the antibody or antigen-binding fragment comprises a light chain CDRL1 having an amino acid sequence that is at least 70%, 75%, 80%, 85%, 90%, 95%, or 100% identical to SEQ ID NO.: 35. In certain embodiments, the antibody or antigen-binding fragment comprises a light chain CDRL2 having an amino acid sequence that is at least 70%, 75%, 80%, 85%, 90%, 95%, or 100% identical to SEQ ID NO.: 36. In certain embodiments, the antibody or antigen-binding fragment comprises a light chain CDRL3 having an amino acid sequence that is at least 70%, 75%, 80%, 85%, 90%, 95%, or 100% identical to SEQ ID NO.: 37. In certain embodiments, the antibody or antigen-binding fragment comprises CDRL1, CDRL2, and CDRL3 having amino acid sequences that are at least 70%, 75%, 80%, 85%, 90%, 95%, or 100% identical to SEQ ID NO.: 35, SEQ ID NO.: 36, and SEQ ID NO.: 37, respectively.

In certain embodiments, the antibody or antigen-binding fragment comprises humanized sequences. In certain embodiments, the antibody or antigen-binding fragment comprises a humanized light chain protein sequence comprising an amino acid sequence that is at least 70%, 75%, 80%, 85%, 90%, 95%, or 100% identical to SEQ ID NO.: 38.

In certain embodiments, the antibody or antigen-binding fragment comprises heavy/light chain protein sequences comprising amino acid sequences that are at least 70%, 75%, 80%, 85%, 90%, 95%, or 100% identical to the amino acid sequences selected from the group consisting of SEQ ID NO.: 34/SEQ ID NO.: 30 and SEQ ID NO.: 34/SEQ ID NO.: 38. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 34/SEQ ID NO.: 30. In certain embodiments, the antibody or antigen-binding fragment comprises heavy/light chain protein sequences comprising amino acid sequences that are at least 70%, 75%, 80%, 85%, 90%, 95%, or 100% identical to SEQ ID NO.: 34/SEQ ID NO.: 38.

For any of the antibody or antigen-binding fragments disclosed herein, in certain embodiments, the antibody or antigen-binding fragment comprises a heavy chain variable domain sequence that is truncated. In certain embodiments, the antibody or antigen-binding fragment comprises a heavy chain variable domain sequence that is truncated to remove one or more heavy chain complementarity determining regions. In certain embodiments, the antibody or antigen-binding fragment comprises a heavy chain variable domain sequence that is truncated to remove all three heavy chain complementarity determining regions.

For any of the antibody or antigen-binding fragments disclosed herein, in certain embodiments, the antibody or antigen-binding fragment comprises one, two, or three heavy chain complementarity determining regions. In certain embodiments, the antibody or antigen-binding fragment comprises up to one, up to two, or up to three heavy chain complementarity determining regions. In certain embodiments, the antibody or antigen-binding fragment comprises at least one, at least two, or at least three heavy chain complementarity determining regions. In certain embodiments, the antibody or antigen-binding fragment does not have heavy chain complementarity determining regions.

Non-limiting examples of antibodies, and derivatives thereof, that can be used in the disclosed methods include chimeric, human, humanized, primatized (CDR-grafted), veneered or single-chain antibodies, as well as functional binding fragments of antibodies. Antigen-binding fragments, or portions thereof, include, but are not limited to, Fv, Fab, Fab′ and F(ab′)₂. Such fragments can be produced by enzymatic cleavage or by recombinant techniques.

In certain embodiments, the antibody is an immunoglobulin. In certain embodiments, the antibody comprises an IgA, IgD, IgE, IgG, and/or IgM. In certain embodiments, the antibody has a K_{d} of at most about 10⁶ M, about 10⁷ M, about 10⁸ M, about 10⁹M, about 10¹⁰ M, about 10¹¹ M, about 10¹² M or less.

In certain embodiments, the antibody or antigen-binding fragment binds specifically to at least one phosphotyrosine selected from the list consisting of pY118/pY120, pY118, pY120, pY342, pY375, and pY391. In certain embodiments, the antibody or antigen-binding fragment specifically binds to pY118/pY120. In certain embodiments, the antibody or antigen-binding fragment binds to pY118/pY120 but does not bind to pY118, pY120, pY342, pY375, or pY391. In certain embodiments, the antibody or antigen-binding fragment does not bind to unphosphorylated extracellular domain of PDGFRA.

### 4. Diagnostic Methods and Methods of Treatment

The presently disclosed subject matter provides methods for assessing whether a subject having cancer is at increased risk of developing progressive disease. The presently disclosed subject matter further provides methods of treating subjects at an increased risk of developing progressive disease. “Increased risk,” as used herein, means at higher risk than subjects lacking a MAN2A1-FER fusion gene; in certain non-limiting embodiments, the risk is increased such that progressive cancer occurs in more than 50%, more than 60% or more than 70% of individuals bearing said fusion gene in one or more cells of their cancer.

Non-limiting examples of cancers include prostate cancer, breast cancer, liver cancer, hepatocarcinoma, hepatoma, lung cancer, non-small cell lung cancer, cervical cancer, endometrial cancer, pancreatic cancer, ovarian cancer, gastric cancer, thyroid cancer, glioblastoma multiforme, colorectal cancer, sarcoma, diffuse large B-cell lymphoma and esophageal adenocarcinoma. In certain embodiments, the cancer is not prostate cancer. In certain embodiments, the cancer is not lung adenocarcinoma, glioblastoma multiforme or hepatocellular carcinoma. In certain embodiments, the target of treatment is a pre-malignant or neoplastic condition involving lung, cervix, endometrium, pancreas, ovary, stomach, thyroid, glia, intestine, esophagus, muscle or B cells. In certain embodiments, the target of treatment is a cell that carries the fusion gene, MAN2A1-FER.

### 4.1. Fusion Gene Detection

The presently disclosed subject matter provides means for determining the presence of a MAN2A1-FER fusion gene in a sample of a subject. A “patient” or “subject,” as used interchangeably herein, refers to a human or a non-human subject. Non-limiting examples of non-human subjects include non-human primates, dogs, cats, mice, etc. In certain embodiments, the subject was not previously diagnosed as having cancer. In certain embodiments, the subject was previously diagnosed as having cancer.

In certain non-limiting embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample can be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid or interstitial fluid), or cells from the individual, including circulating cancer cells. In certain non-limiting embodiments, the sample is obtained from a cancer. In certain embodiments, the sample can be a “biopsy sample” or “clinical sample,” which are samples derived from a subject. In certain embodiments, the sample includes one or more cancer cells from a subject. In certain embodiments, MAN2A1-FER can be detected in one or more samples obtained from a subject.

In certain embodiments, MAN2A1-FER is detected by antibody binding analysis such as, but not limited to, Western Blot analysis and immunohistochemistry. In certain embodiments, the MAN2A1-FER is detected using an antibody or antigen-binding fragment disclosed herein.

### 4.2. Diagnostic Methods for Assessing the Risk of Progressive Cancer

The presently disclosed subject matter provides for methods of determining whether a subject is at increased risk of manifesting progressive cancer. In certain non-limiting embodiments, the method of determining whether a subject is at increased risk of manifesting progressive cancer comprises obtaining a sample from the subject, determining whether a subject is at increased risk of manifesting progressive cancer comprising determining whether the sample contains a fusion gene, wherein the presence of the fusion gene in the sample is indicative that the subject is at increased risk of manifesting progressive cancer. In certain embodiments, the fusion gene is MAN2A1-FER. In certain embodiments, determining whether the sample contains a fusion gene comprises contacting the sample with the antibody or antigen-binding fragment disclosed herein.

Molecular imaging, e.g., PET scan, can be used to determine the presence of cancer expressing MAN2A1-FER fusion gene in a subject. In certain embodiments, cancer expressing MAN2A1-FER is detected through molecular imaging using a tracer compound, e.g., a radioactive tracer, that is conjugated to antibodies that bind to MAN2A1-FER. In certain embodiments, the tracer compound is conjugated to the antibody or antigen-binding fragment disclosed herein.

### 4.3. Methods of Treatment

The presently disclosed subject matter provides methods of treating a subject, e.g., a subject having cancer that carries a MAN2A1-FER fusion gene or a subject that has one or more cells that comprise a fusion gene, comprises determining the presence of a MAN2A1-FER fusion gene in a sample obtained from a subject, where if a MAN2A1-FER fusion gene is detected in the sample then providing treatment to the subject. In certain embodiments, the treatment comprises administering to the subject a therapeutically effective amount of an antibody or antigen-binding fragment disclosed herein. In certain embodiments, the antibody or antigen-binding fragment can be administered to produce an anti-cancer effect in a subject. In certain embodiments, the antibody or antigen-binding fragment binds specifically to phosphorylated PDGFRA. In certain embodiments, the antibody or antigen-binding fragment binds specifically to at least one phosphotyrosine selected from the list consisting of pY118/pY120, pY118, pY120, pY342, pY375, and pY391. In certain embodiments, the antibody or antigen-binding fragment specifically binds to pY118/pY120. In certain embodiments, the antibody or antigen-binding fragment binds to pY118/pY120 but does not bind to pY118, pY120, pY342, pY375, or pY391. In certain embodiments, the antibody or antigen-binding fragment does not bind to unphosphorylated extracellular domain of PDGFRA. In certain embodiments, the antigen-binding fragment is a Fv, Fab, Fab′, or F(ab′)₂.

An anti-cancer agent can be any molecule, compound chemical or composition that has an anti-cancer effect. Anti-cancer agents include, but are not limited to, chemotherapeutic agents, radiotherapeutic agents, cytokines, anti-angiogenic agents, apoptosis-inducing agents or anti-cancer immunotoxins. In certain non-limiting embodiments, the antibody or antigen-binding fragment can be administered in combination with one or more anti-cancer agents. “In combination with,” as used herein, means that the antibody or antigen-binding fragment and the one or more anti-cancer agents are administered to a subject as part of a treatment regimen or plan. This term does not require that the antibody or antigen-binding fragment and one or more anti-cancer agents are physically combined prior to administration nor that they be administered over the same time frame. In certain embodiments, the antibody or antigen-binding fragment is conjugated to an anti-cancer agent.

An “anti-cancer effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse or progression-free survival in a patient diagnosed with cancer. Similarly, an “anti-neoplastic effect” refers to one or more of a reduction in aggregate neoplastic cell mass, a reduction in neoplastic cell growth rate, a reduction in neoplasm progression (e.g., progressive de-differentiation or epithelial to mesenchymal transition), a reduction in neoplastic cell proliferation, a reduction in neoplasm mass, a reduction in neoplasm volume, and/or a reduction in neoplasm growth rate.

A “therapeutically effective amount” refers to an amount that is able to achieve one or more of the following: an anti-cancer effect, an anti-neoplastic effect, a prolongation of survival and/or prolongation of period until relapse.

In certain embodiments, the presently disclosed subject matter provides a method for lengthening the period of survival of a subject having a cancer. In certain embodiments, the method comprises determining the presence of one or more fusion genes in a sample of the subject, where if one or more fusion genes are detected in the sample then administering to the subject a therapeutically effective amount of an antibody or antigen-binding fragment. In certain embodiments, the period of survival of a subject having cancer can be lengthened by about 1 month, about 2 months, about 4 months, about 6 months, about 8 months, about 10 months, about 12 months, about 14 months, about 18 months, about 20 months, about 2 years, about 3 years, about 5 years or more using the disclosed methods.

If it is determined that the patient is at increased risk for progressive cancer, a healthcare provider can recommend and/or perform frequent monitoring of the patient, e.g. ultrasound, CT scan, MRI, or PET scan, and/or recommend and/or perform a therapeutic procedure, for example but not limited to surgical excision, radiotherapy, chemotherapy, and/or antibody therapy. In certain embodiments, the therapeutic procedure comprises administering a therapeutically effective amount of an antibody or antigen-binding fragment disclosed herein.

If it is determined that the patient is at increased risk for progressive cancer, a healthcare provider can optionally take the further step of recommending and/or performing frequent monitoring of the patient for recurrence, e.g., ultrasound, CT scan, MRI, or PET scan, and/or recommending and/or performing a therapeutic procedure, for example but not limited to surgical excision, radiotherapy, chemotherapy, and/or antibody therapy.

### 5. Pharmaceutical Compositions

In certain non-limiting embodiments, the presently disclosed subject matter provides for pharmaceutical formulations of the antibodies and antigen-binding fragments disclosed above for therapeutic use. In certain embodiments, a pharmaceutical formulation comprises an antibody or antigen-binding fragment and a pharmaceutically acceptable carrier. “Pharmaceutically acceptable carrier,” as used herein, includes any carrier which does not interfere with the effectiveness of the biological activity of the active ingredients and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioabsorbable matrix materials, implantation elements containing the antibody or antigen-binding fragment and/or any other suitable vehicle, delivery or dispensing means or material. Such carriers can be formulated by conventional methods and can be administered to the subject. In certain embodiments, the pharmaceutical acceptable carrier can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, a suitable pharmaceutically acceptable carrier can include one or more of water, saline, phosphate buffered saline, dextrose, glycerol, ethanol or combinations thereof.

In certain non-limiting embodiments, the pharmaceutical formulations of the presently disclosed subject matter can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical formulation can be a solid dosage form. In certain embodiments, the tablet can be an immediate release tablet. Alternatively or additionally, the tablet can be an extended or controlled release tablet. In certain embodiments, the solid dosage can include both an immediate release portion and an extended or controlled release portion.

In certain embodiments, the pharmaceutical formulations of the presently disclosed subject matter can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, formulations of the presently disclosed subject matter can be administered to the patient intravenously in a pharmaceutically acceptable carrier such as physiological saline.

In certain embodiments, the pharmaceutical formulations suitable for use in the presently disclosed subject matter can include formulations where the active ingredients are contained in a therapeutically effective amount. The therapeutically effective amount of an active ingredient can vary depending on the active ingredient, formulation used, the cancer and its severity, and the age, weight, etc., of the subject to be treated. In certain embodiments, a patient can receive a therapeutically effective amount of an antibody or antigen-binding fragment and/or agent disclosed herein in single or multiple administrations of one or more formulations, which can depend on the dosage and frequency as required and tolerated by the patient.

In certain non-limiting embodiments, the antibodies, antigen-binding fragments, and/or agents described above can be used alone or in combination with one or more anti-cancer agents. As noted above, “in combination with” means that the antibody or antigen-binding fragment and the one or more anti-cancer agents are administered to a subject as part of a treatment regimen or plan. In certain embodiments, being used in combination does not require that the antibody or antigen-binding fragment and the one or more anti-cancer agents are physically combined prior to administration or that they be administered over the same time frame. Accordingly, a second anti-cancer agent can be administered prior to, concurrently with, or subsequent to, administration of one or more doses of the antibody or antigen-binding fragment.

### 5.1. Antibody-Directed Nanoparticles

The presently disclosed subject matter relates to antibody-directed nanoparticles comprising an antibody or antigen binding fragment disclosed herein. Nanoparticles can be functionalized to bind biological molecules (e.g., a ligand or an antibody) targeting a specific tissue (e.g., cancer cells). When bound to the antibodies or antigen-binding fragments of the present disclosure, the delivery of nanoparticles can be enhanced to cells exhibiting the phosphorylated extracellular domain of PDGFRA. This additionally enhances the delivery of cargo encapsulated within the nanoparticles, e.g., therapeutic agents, to such cells.

The surface functionalization of nanoparticles can be based on the use of homo- or hetero-bifunctional cross linkers to the aim to add an organic functional group (e.g., R—NH2, R—COOH, etc.), useful to bind biological molecules (e.g., a ligand or an antibody). In certain embodiments, the functionalization of the surface of the nanoparticles can be achieved using covalent or non-covalent conjugation.

In certain embodiments, the nanoparticle can include one or more lipids. In certain embodiments, the lipids can be neutral, anionic or cationic at physiological pH. In certain embodiments, the lipids can be sterols. For example, in certain embodiments, the lipid nanoparticle include cholesterol, phospholipids and sphingolipids. In certain embodiments, the nanoparticles comprise PEGylated derivatives of the neutral, anionic, and cationic lipids.

In certain embodiments, the nanoparticles include polymers. In certain embodiments, the polymer can be amphiphilic, hydrophilic, or hydrophobic. In certain embodiments, the polymer can be biocompatible, e.g., the polymer does not induce an adverse and/or inflammatory response when administered to a subject. For example, without limitation, a polymer can be selected from polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), poly(lactide-co-glycolide) (PLG), polycaprolactone, copolymers, or derivatives including these and/or other polymers. In certain embodiments, the polymer includes PEG. In certain embodiments, the polymer includes poly(lactide-co-glycolide) (PLG).

In certain embodiments, the nanoparticles can have a diameter of less than 1000 m, e.g., from about 10 μm to about 200 μm. In certain embodiments, the nanoparticles can have a diameter of from about 10 μm to about 90 μm, from about 20 μm to about 80 μm, from about 60 μm to about 120 μm, from about 70 μm to about 120 μm, from about 80 μm to about 120 μm, from about 90 μm to about 120 μm, from about 100 μm to about 120 μm, from about 60 μm to about 130 μm, from about 70 μm to about 130 μm, from about 80 μm to about 130 μm, from about 90 μm to about 130 μm, from about 100 μm to about 130 μm, from about 110 μm to about 130 μm, from about 60 μm to about 140 μm, from about 70 μm to about 140 μm, from about 80 μm to about 140 μm, from about 90 μm to about 140 μm, from about 100 μm to about 140 μm, from about 110 μm to about 140 μm, from about 60 μm to about 150 μm, from about 70 μm to about 150 μm, from about 80 μm to about 150 μm, from about 90 μm to about 150 μm, from about 100 μm to about 150 μm, from about 110 μm to about 150 μm, or from about 120 μm to about 150 μm. In certain embodiments, the nanoparticles can have a diameter of from about 1 μm to about 30 μm, from about 2 μm to about 30 μm, from about 5 μm to about 30 μm, from about 7 μm to about 30 μm, from about 10 μm to about 30 μm, from about 12 μm to about 30 μm, from about 15 μm to about 30 μm, from about 20 μm to about 30 μm, from about 5 μm to about 20 μm, from about 8 μm to about 20 μm, from about 10 μm to about 20 μm, from about 12 μm to about 20 μm, from about 15 μm to about 20 μm, or from about 10 μm to about 15 μm. In certain embodiments, the nanoparticles can have a diameter of from about 10 μm to about 20 μm

In certain embodiments, the nanoparticles can have a diameter of from about 10 nm to about 1000 nm, from about 50 nm to about 1000 nm, from about 100 nm to about 1000 nm, from about 150 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 500 nm to about 900 nm, from about 600 nm to about 900 nm, from about 700 nm to about 900 nm, from about 800 nm to about 900 nm, from about 100 nm to about 200 nm, from about 100 nm to about 300 nm, from about 100 nm to about 400 nm, from about 600 nm to about 800 nm, or from about 700 nm to about 800 nm. In certain embodiments, the nanoparticles can have a diameter of from about 10 nm to about 100 nm, from about 20 nm to about 100 nm, from about 50 nm to about 100 nm, from about 70 nm to about 100 nm, from about 110 nm to about 200 nm, from about 120 nm to about 100 nm, from about 150 nm to about 200 nm, from about 200 nm to about 300 nm, or from about 250 nm to about 300 nm.

In certain embodiments, the nanoparticle can include one or more lipids. In certain embodiments, the lipids can be neutral, anionic or cationic at physiological pH. In certain embodiments, the lipids can be sterols. For example, in certain embodiments, the lipid nanoparticle include cholesterol, phospholipids and sphingolipids. In certain embodiments, the nanoparticles comprise PEGylated derivatives of the neutral, anionic, and cationic lipids. The incorporation of PEGylated derivatives can improve the stability of the nanoparticles.

In certain embodiments, the nanoparticle includes one or more therapeutic agent. In certain embodiments, the one or more therapeutic agent is attached to the surface of the nanoparticle. In certain embodiments, the one or more therapeutic agent is encapsulated into the nanoparticle. Non-limiting examples of therapeutic agents include antibodies, antigen-binding fragments, cytokines, chemokines, antibiotics, chemotherapeutic agents, nucleic acids, siRNA, proteins, radioisotopes. In certain embodiments, the one or more therapeutic agent comprises one or more selected from the group consisting of doxorubicin, monomethyl auristatin E (MMAE), DM1, and combinations thereof. In certain embodiments, the one or more therapeutic agent comprises doxorubicin. In certain embodiments, the one or more therapeutic agent comprises MMAE. In certain embodiments, the one or more therapeutic agent comprises DM1.

In certain embodiments, the nanoparticles comprise nucleic acids encoding Cas9 and one or more gRNAs. In certain embodiments, the one or more gRNAs are directed to genes selected from the group consisting of STAT3a, PDL1, PDL2, and combinations thereof. In certain embodiments, the one or more nucleic acids are expressed under the control of an inducible promoter. In certain embodiments, the one or more nucleic acids comprise Casepase-9 (Casp9). In certain embodiments, the expression of Casp9 is induced in the presence of AP1903. In certain embodiments, the one or more nucleic acids encode a protein that is fused to reporter. In certain embodiments, the reporter is a luciferase or eGFP construct.

### 6. Kits

The presently disclosed subject matter further provides kits for detecting a MAN2A1-FER fusion genes disclosed herein and/or for carrying any one of the above-listed detection and therapeutic methods. Types of kits include, but are not limited to, packaged fusion gene-specific probe and primer sets (e.g., TaqMan probe/primer sets), arrays/microarrays, antibodies, which further contain one or more probes, primers, or other reagents for detecting a MAN2A1-FER fusion gene.

In certain non-limiting embodiments, a kit is provided comprising one or more nucleic acid primers or probes and/or antibody probes for use in carrying out any of the above-listed methods. Said probes can be detectably labeled, for example with a biotin, colorimetric, fluorescent or radioactive marker. A nucleic acid primer can be provided as part of a pair, for example for use in polymerase chain reaction. In certain non-limiting embodiments, a nucleic acid primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length. A nucleic acid probe can be an oligonucleotide probe and/or a probe suitable for FISH analysis.

In certain embodiments, the kit comprises an antibody or antigen-binding fragment disclosed herein. In certain embodiments, the antibody or antigen-binding fragment does not bind to pY118, pY120, pY342, pY375, or pY391. In certain embodiments, the antibody or antigen-binding fragment specifically binds to pY118/pY120. In certain embodiments, the antibody or antigen-binding fragment does not bind to unphosphorylated extracellular domain of PDGFRA. In certain embodiments, the antigen-binding fragment is a Fv, Fab, Fab′, or F(ab′)₂. In certain embodiments, the kit comprises antibody-directed nanoparticles comprising an antibody or antigen binding fragment as disclosed herein.

### EXAMPLES

The present disclosure will be better understood by reference to the following Examples, which are provided as exemplary of the presently disclosed subject matter, and not by way of limitation.

### Example 1: Immuno-Targeting the Ectopic Phosphorylation Sites of PDGFRA Generated by MAN2A1-FER Fusion in Hepatocellular Carcinoma

Hepatocellular carcinoma (HCC) is one of the most lethal cancers for humans. MAN2A1-FER is one of the most frequent oncogenic fusion genes in the HCC. MAN2A1-FER ectopically phosphorylates the extracellular domains of PDGFRA, MET, AXL, and N-cadherin. The ectopic phosphorylation of these transmembrane proteins lead to the activation of their kinase activities and initiate the activation cascades of their downstream signaling molecules. A panel of mouse monoclonal antibodies was developed to recognize the ectopic phosphorylation sites of PDGFRA. The analyses showed that these antibodies bound to the specific phosphotyrosine epitopes in the extracellular domain of PDGFRA with high affinity and specificity. The treatment of MAN2A1-FER positive cancer HUH7 with one of the antibodies called 2-3B-G8 led to the deactivation of cell growth signaling pathways and cell growth arrest, while had minimal impact on HUH7ko cells where MAN2A1-FER expression was disrupted. The treatment of 2-3B-G8 antibody also led to a large number of cell deaths of MAN2A1-FER positive cancer cells such as HUH7, HEPG2, SNU449, etc., while the same treatment had no impact on HUH7ko cells. When severe combined-immunodeficiency mice xenografted with HEPG2 or HUH7 were treated with Monomethyl auristatin E (MMAE) conjugated 2-3B-G8 antibody, it slowed the progression of tumor growth, eliminated the metastasis, and reduced the mortality, in comparison with the controls. Therefore, targeting the cancer-specific ectopic phosphorylation sites of PDGFRA induced by MAN2A1-FER is an effective treatment for liver cancer.

### Methods

Cell lines. The cell lines were purchased from the American Type Culture Collection (ATCC, Manassas, Virginia) and were cultured and maintained following the recommendations of the manufacturer. They were authenticated every 6 months and were free of Mycoplasma. Rabbit anti-FER antibodies were purchased from Invitrogen Inc, Waltham, Massachusetts (PA5-49788).

RNA extraction, cDNA synthesis, TaqMan RT-PCR, and breakpoint Taqman-PCR. Total RNA was extracted from cell pellets using TRIzol (Invitrogen, CA). Two micrograms of RNA were used to synthesize the first-strand cDNA with random hexamer primers and Superscript II™ (Invitrogen, CA). One microliter of each cDNA sample was used for TaqMan PCR (Eppendorf realplex mastercycler and AppliedBiosystems QuantStudio 3) with 50 heating cycles at 94° C. for 30 seconds, 61° C. for 30 seconds, and 72° C. for 30 seconds using the primer sequences AGCGCAGTTTGGGATACAGCA (SEQ ID NO.: 39) and CTTTAATGTGCCCTTATATACTTCACC (SEQ ID NO: 40) and the TaqMan probe: 5′/56-FAM/TCAGAAACAGCCTATGAGGGAAATT/3IABkFQ/3′ (SEQ ID NO.: 41) in a thermocycler (QuantStudio 3 real-time PCR system, Thermofisher, Inc or Mastercycler® 6 RealPlex2, Eppendorf, Inc). At least one negative control and a synthetic positive control were included in each reaction batch. The PCR products were gel purified, and Sanger sequencing was performed on all positive samples. The genome DNA of cell lines was extracted using QIAamp DNA kit (Qiagen, Inc). One microgram of genome DNA was used for TaqMan PCR with the following conditions: 50 heating cycles at 94° C. for 30 seconds, 61° C. for 30 seconds, and 72° C. for 30 seconds using the primer sequences CTCAAACTCCTGACCCCGTGA (SEQ ID NO.: 42) and GAACACAAACCCTTAGGGGGC (SEQ ID NO.: 43) and the following TaqMan probe: 5′/56-FAM/CCACCTTCTAGCTATTGAGTAGC/3IABkFQ//3′ (SEQ ID NO.: 44). All positive PCR results were Sanger-sequenced. ZEN internal quenchers were placed between the ninth and tenth bases from the 5′ end of the TaqMan probes.

Generation of vectors expressing HisTAG-PDGFRA, HisTAG-MET, HisTAG-N-cadherin and HisTAG-AXL. For pET28-HisTAG-ΔPDGFRA^{aa1-528} construction, a PCR using the primers indicated in Table 2 and cDNA of PDGFRA (Harvard University, DF/HCC) as the template, in the following condition: 1 cycle of 94° C. for 1 minute, followed by 35 cycles at 94° C. for 30 seconds, 68° C. for 30 seconds, and 72° C. for 3 minutes. The gel-purified PCR products were digested with EcoR1 and NOT1 and ligated into the similarly digested pET28(a)+ vector to generate the pET28-HisTAG-ΔPDGFRA^{aa1-528} vector. To construct pCMV13-HisTAG-ΔPDGFRA^{aa1-528} expression vector, a PCR was performed using the primer pair indicated in Table 2 on PDGFRA cDNA template. The PCR product was then digested with HindIII and Xba1. The digested product was ligated to a similarly digested pCMV13 vector to create pCMV13-ΔHisTAG-PDGFRA^{aa1-528} vector. Similar construction procedure was performed for pET28-HisTAG-ΔMET^{aa1-646} pCMV13-HisTAG-ΔMET^{aa1-646}, pET28-HisTAG-ΔAXL^{aa1-451}, pCMV13-HisTAG-ΔAXL^{aa1-451}, pET28-HisTAG-ΔCDH2^{aa1-724}, pCMV13-HisTAG-ΔCDH2^{aa1-724} using the respective primers and cDNA templates indicated in Table 2.

TABLE 2
Primers for vector construction.

| Vector | Primer Pair | cDNA |
|---|---|---|
| pET28-HisTAG- | AAGCTTGAATTCATGGGGACTTCCCATCCGG | PDGFRA |
| ΔPDGFRA^{aa1-528} | CGT (SEQ ID NO.: 45) | |
| | TCTAGAGCGGCCGCAGCCACCGTGAGTTCA | |
| | GAACG (SEQ ID NO.: 46) | |
| pCMV13-HisTAG- | AAGCTTGAATTCCATCATCATCATCATCACAT | PDGFRA |
| ΔPDGFRA^{aa1-528} | GGGGACTTCCCATCCGGCGT (SEQ ID NO.: 47) | |
| | TCTAGAGCGGCCGCAGCCACCGTGAGTTCA | |
| | GAACG (SEQ ID NO.: 48) | |
| pET28-HisTAG- | AAGCTTGAATTCATGAAGGCCCCCGCTGTGC | MET |
| ΔMET^{aa1-646} | TT (SEQ ID NO.: 49) | |
| | TCTAGAGCGGCCGCTGTCCCGTGGCCATTTG | |
| | AAAT (SEQ ID NO.: 50) | |
| pCMV13-HisTAG- | AAGCTTGAATTCCATCATCATCATCATCACAT | MET |
| ΔMET^{aa1-646} | GAAGGCCCCCGCTGTGCTT (SEQ ID NO.: 51) | |
| | TCTAGAGCGGCCGCTGTCCCGTGGCCATTTG | |
| | AAAT (SEQ ID NO.: 52) | |
| pET28-HisTAG- | AAGCTTGAATTCATGGCGTGGCGGTGCCCCA | AXL |
| ΔAXL^{aa1-451} | GG (SEQ ID NO.: 53) | |
| | TCTAGAGCGGCCGCCCACCAGGGCCACGAG | |
| | AAGGC (SEQ ID NO.: 54) | |
| pCMV13-HisTAG- | AAGCTTGAATTCCATCATCATCATCATCACAT | AXL |
| ΔAXL^{aa1-451} | GGCGTGGCGGTGCCCCAGG (SEQ ID NO.: 55) | |
| | TCTAGAGCGGCCGCCCACCAGGGCCACGAG | |
| | AAGGC (SEQ ID NO.: 56) | |
| pET28-HisTAG- | AAGCTTGAATTCATGTGCCGGATAGCGGGAG | CDH2 |
| ΔCDH2^{aa1-724} | CG (SEQ ID NO.: 57) | |
| | TCTAGAGCGGCCGCGGCACCGGTGCCAAGC | |
| | CCCGC (SEQ ID NO.: 58) | |
| pCMV13-HisTAG- | AAGCTTGAATTCCATCATCATCATCATCACAT | CDH2 |
| ΔCDH2^{aa1-724} | GTGCCGGATAGCGGGAGCG (SEQ ID NO.: 59) | |
| | TCTAGAGCGGCCGCGGCACCGGTGCCAAGC | |
| | CCCGC (SEQ ID NO.: 60) | |

In vitro kinase assay. E. coli harboring GST, GST-MAN2A1-FER, and GST-FER were grown overnight in room temperature. The recombinant proteins were induced with 1 mM IPTG for 4 hours. GST, GST-MAN2A1-FER, and GST-FER were purified by a glutathione column and diluted to 1 ng/l with 1× kinase assay buffer provided by the manufacturer (Cell Signaling, Inc, Danvers, MA). This was followed by combining 25 μl GST (50 ng) or GST-MAN2A1-FER (50 ng) or GST-FER (50 ng) and 25 μl substrate (3 μM poly EY[4:1], 1 μg HisTAG-ΔEGFR^{aa1-650}, 1 μg HisTAG-ΔPDGFRA^{aa1-528}, 1 μg HisTAG-ΔMET^{aa1-646}, 1 μg HisTAG-ΔCDH2^{aa1-724}, or 1 μg HisTAG-ΔAXL^{aa1-451}). The solutions were incubated at 37° C. for 60 minutes. The reactions were terminated by adding 25 μl of 2N NaOH stop solution to each reaction well. The kinase activities were quantified using the kit and protocols of ADP-Glo™ Kinase Assay from Promega, Inc, Madison, WI.

Generation of monoclonal antibodies specific for phospho-HisTAG-ΔPDGFRAaa1-528. The phospho-HisTAG-ΔPDGFRA^{aa1-528} recombinant protein expressed from HUH7 was purified through HisTAG column and used as an immunogen. Five female CL6/B6 mice (8 weeks old) were each immunized through intraperitoneal injection of 10 μg purified phospho-HisTAG-ΔPDGFRA^{aa1-528} protein (from pCMV13-HisTAG-ΔPDGFRA^{aa1-528} transformed HUH7) that had been emulsified in 0.1 ml of Freund's adjuvant. These mice were subsequently boosted twice with 10 μg phospho-HisTAG-ΔPDGFRA^{aa1-528} in Freund's incomplete adjuvant at 21-day intervals. A mouse with high reactivity (>1:100,000 by Enzyme-linked immunosorbent assay) was chosen and injected with 20 μg phospho-HisTAG-ΔPDGFRA^{aa1-528} in 0.5 ml PBS 3 days prior to hybridoma fusion.

Once the animal was sacrificed, the spleen of the immunized mouse was removed. To separate spleen cells, the medium was then gently flushed through the spleen from different angles. Spleen cells were isolated and fused with SP2/0-Ag-14 myeloma cells in the presence of polyethylene glycol (PEG 1500) (Sigma) to produce hybridomas. Fused cells were cultured and selected in RPMI supplemented with 20% FBS, 100 U/ml penicillin, 100 mg/ml streptomycin (Bioidea), 1× non-essential amino acids, 1 mM sodium pyruvate (Gibco), and 1× hypoxanthine, aminopterin, and thymidine solution (HAT, 50 stock solution, Sigma). The fused cells were plated onto 96-well plates and screened for monoclonal antibody production by ELISA starting on day 18 post-fusion. The ELISA-positive hybridomas were diluted three times by limiting dilution technique at 50, 10, and 1 cells/96-well plates. The resultant stable colonies were expanded into 25-cm² Falcon flasks. Hybridoma supernatants were screened for anti-phospho-HisTAG-ΔPDGFRA^{aa1-528} antibody by ELISA and immunoblot analyses. The positive clones were screened for reactivity against unphosphorylated HisTAG-ΔPDGFRA^{aa1-528}. Clones positive for phospho-HisTAG-ΔPDGFRA^{aa1-528} (from pCMV13-HisTAG-ΔPDGFRA^{aa1-528} transformed HUH7) but negative for unphosphorylated HisTAG-ΔPDGFRA^{aa1-528} (from E. coli or HUH7ko) were selected. These clones were further confirmed by immunofluorescence staining and immunoblot analyses. Five positive clones were selected for further assays. For 2-3B-G8-MMAE conjugation, the antibody MMAE conjugation kit (Mosiac, Inc) was used. The conjugation procedure followed the recommendation by the manufacturer.

Enzyme-linked immunosorbent assay (ELISA). Briefly, microtiter 96 well polystyrene plate was coated with 20 mg/ml of phospho-HisTAG-ΔPDGFRA^{aa1-528} dissolved in bicarbonate/carbonate coating buffer (100 mM, pH 9.2) and then incubated at 4° C. overnight. After washing with PBS containing 0.05% Tween 20 (PBS-T), nonspecific sites were blocked with 2% bovine serum albumin (BSA) at 37° C. for 2 h. The wells were then washed twice. The diluted sera were added to antigen-coated wells. After incubation for 2 h at room temperature, wells were washed 3 times with PBS-T. Next, 100 ml of goat anti-mouse Ig conjugated to horseradish peroxides (HRP, 1:5000) was added to each well and incubated for 1 h at 37° C. After 3 times of washing, 100 ml of tetramethylbenzidine (TMB) substrate solution was added, and the plate was incubated for 15 min in the dark. The reaction was stopped with H₂SO₄ (0.2 M). The optical density (OD) was measured by an ELISA reader at 450 nm. The mouse with the highest serum titer was selected. Hybridoma culture supernatant was tested similarly. For antibody-antigen-binding assays, the antibody at various concentrations (78 ng-10 μg/ml) was dissolved in 0.1 M potassium phosphate, 2 mM EDTA, pH 7.8, supplemented with 10 mg/ml BSA. The solution was transferred and incubated for 1 h at 20° C. into the wells of a microtitration plate previously coated with phospho-HisTAG-ΔPDGFRA^{aa1-528} (at 100 μg/well in 50 mM sodium carbonate, pH 9.6 for 15 h at 4° C.) for ELISA assay. The binding affinity was calculated by Scatchard analysis.

BrdU cell cycle assays. HUH7 cells or their MAN2A1-FER knockout counterparts treated with 2-3B-G8 antibody or IgG were used in the cell cycle analyses. A FITC-BrdU flow kit (BD Biosciences) was used. Cells were synchronized by culture in FBS-free medium for 48 hours, followed by replenishment with medium containing 10% FBS and BrdU for 4 hours. Cells were harvested for analysis with a FITC-BrdU antibody and propidium iodide (PI) nuclear staining (BD Biosciences). The distribution of cells in different cell cycle phases was analyzed by flow cytometry (BD FACSCalibur).

Cell death assay in 2-3B-G8 or IgG-treated cancer cells. Cancer cell lines carrying MAN2A1-FER or their knockout counterparts were used. Cells were treated with 2-3B-G8 antibody or with non-specific mouse IgG as the control. Cell cultures at 70-80% confluence were treated with these antibodies for 18-24 hours. Cells were then harvested for cell death analysis with a PE-Annexin V apoptosis assay kit (BD Biosciences). Cells were resuspended in 100 μl of annexin V binding buffer (Invitrogen) and incubated with 5 μl of phycoerythrin (PE)-conjugated annexin V and 5 μl of propidium iodide for 20 minutes in the dark at room temperature. The binding assays were terminated by the addition of 400 μl of annexin V binding buffer. FACS analysis was performed using a BD FACSCalibur (BD Sciences, San Jose, CA). Ten thousand cells were acquired and sorted. WinMDI 2.9 software (freeware from Joseph Trotter) was used to analyze the data.

Immunoblotting. Cells were lysed in RIPA buffer for 10 minutes on ice. The crude lysates were then centrifuged at 10,000 rpm for 10 minutes at 4° C. Supernatant/protein extract was obtained. For Western blotting, the sodium dodecyl sulfate-polyacrylamide electrophoresis resolved proteins were transferred to a hydrophilic polyvinylidene fluoride membrane. The membrane was blocked by incubating in a blocking solution (PBS containing 5% skim milk and 0.05% Tween-20) for 1.5 h at room temperature and then probed with hybridoma supernatant containing antibodies or the other indicated antibody overnight at 4° C. After three PBS-T washes, the membrane was incubated with HRP-conjugated anti-mouse IgG (BD Bioscience; 1:4000 in PBS-T) for 1 h at room temperature. After washing with PBS-T, protein detection was achieved with the enhanced chemiluminescence (ECL) system (Bio-Rad). For benzoic acid partial digestion analyses, 0.5 mg of phospho-HisTAG-ΔPDGFRA^{aa1-528} were incubated with 10 mg/ml Benzoic acid) at 22° C. for 10 min. The digested products were then purified through HisTAG column. The purified products were resolved in 15% SDS-PAGE and immunoblotted with antibodies specific for the phospho-N-terminus of PDGFRA.

Dot immunobinding assays were performed by placing the nitrocellulose membrane on a blotting paper. The membrane was then added a drop of peptide (10 ng/μl) listed in Table 3. The membrane was blocked by a blocking solution as above for 1 h at room temperature. The blocked membrane was incubated with 2-3B-G8, 1-3C-F11, 2-3C-C5, 2-6B-D8, 1-4B-B10, or IgG antibody (10 ng/ml) for an hour and washed three times in TBST. After three PBS-T washes, the membrane was incubated with RP-conjugated anti-mouse Ig (BD Bioscience; 1:4000 in PBS-T) for 1 h at room temperature. After washing with PBS-T, protein detection was achieved with the enhanced chemiluminescence (ECL) system (Bio-Rad).

TABLE 3
Peptide sequences corresponding to extracellular domains of PDGFR, MET, AXL,
and CDH2.

| | Peptide | Tyrosine | | |
|---|---|---|---|---|
| Gene | No. | position | Sequence | SEQ ID NO |
| PDGFR | 1 | Y59 | CFGESEVSWQYPMSEEESSDV | SEQ ID NO.: 61 |
| | 2 | Y98/Y101- | SSASAAHTGLYTCYYNHTQTE | SEQ ID NO.: 62 |
| | | 2 | | |
| | 3 | Y118/Y120 | EENELEGRHIYIYVPDPDVAF | SEQ ID NO.: 63 |
| | 4 | Y136 | VAFVPLGMTDYLVIVEDDDSA | SEQ ID NO.: 64 |
| | 5 | Y172 | HNSEGVVPASYDSRQGFNGTF | SEQ ID NO.: 65 |
| | 6 | Y187 | GFNGTFTVGPYICEATVKGKK | SEQ ID NO.: 66 |
| | 7 | Y206 | KKFQTIPFNVYALKATSELDL | SEQ ID NO.: 67 |
| | 8 | Y225 | DLEMEALKTVYKSGETIVVTC | SEQ ID NO.: 68 |
| | 9 | Y249 | NNEVVDLQWTYPGEVKGKGIT | SEQ ID NO.: 69 |
| | 10 | Y273 | EIKVPSIKLVYTLTVPEATVK | SEQ ID NO.: 70 |
| | 11 | Y288 | PEATVKDSGDYECAARQATRE | SEQ ID NO.: 71 |
| | 12 | Y342 | VKHFVVEVRAYPPPRISWLKN | SEQ ID NO.: 72 |
| | 13 | Y375 | TDVEKIQEIRYRSKLKLIRAK | SEQ ID NO.: 73 |
| | 14 | Y391 | IRAKEEDSGHYTIVAQNEDAV | SEQ ID NO.: 74 |
| | 15 | Y405 | VAQNEDAVKSYTFELLTQVPS | SEQ ID NO.: 75 |
| MET | 1 | Y41 | AKSEMNVNMKYQLPNFTAETP | SEQ ID NO.: 76 |
| | 2 | Y69/Y71 | EHHIFLGATNYIYVLNEEDLQ | SEQ ID NO.: 77 |
| | 3 | Y84 | NEEDLQKVAEYKTGPVLEHPD | SEQ ID NO.: 78 |
| | 4 | Y125/Y126 | NINMALVVDTYYDDQLISCGS | SEQ ID NO.: 79 |
| | 5 | Y205 | FFVGNTINSSYFPDHPLHSIS | SEQ ID NO.: 80 |
| | 6 | Y234 | DGFMFLTDQSYIDVLPEFRDS | SEQ ID NO.: 81 |
| | 7 | Y245/Y249 | IDVLPEFRDSYPIKYVHAFES | SEQ ID NO.: 82 |
| | 8 | Y260 | VHAFESNNFIYFLTVQRETLD | SEQ ID NO.: 83 |
| | 9 | Y291 | FCSINSGLHSYMEMPLECILT | SEQ ID NO.: 84 |
| | 10 | Y321 | KEVFNILQAAYVSKPGAQLAR | SEQ ID NO.: 85 |
| | 11 | Y369 | RSAMCAFPIKYVNDFFNKIVN | SEQ ID NO.: 86 |
| | 12 | Y390 | KNNVRCLQHFYGPNHEHCFNR | SEQ ID NO.: 87 |
| | 13 | Y416 | SSGCEARRDEYRTEFTTALQR | SEQ ID NO.: 88 |
| | 14 | Y501 | IVEHTLNQNGYTLVITGKKIT | SEQ ID NO.: 89 |
| | 15 | Y566 | WTQQICLPAIYKVFPNSAPLE | SEQ ID NO.: 90 |
| AXL | 1 | Y115 | RITSLQLSDTGQYQCLVFLGHQTFV | SEQ ID NO.: 91 |
| | 2 | Y132 | QCLVFLGHQTFVSQPGYVGLEGLP | SEQ ID NO.: 92 |
| | 3 | Y140 | VGLEGLPYFLEEPEDRTVAANTPFN | SEQ ID NO.: 93 |
| | 4 | Y253 | LEVAWTPGLSGIYPLTHCTLQAVLS | SEQ ID NO.: 94 |
| | 5 | Y367/Y371 | RAPLQGTLLGYRLAYQGQDTPEVLM | SEQ ID NO.: 95 |
| | 6 | Y409 | GSVSNLTVCVAAYTAAGDGPWSLPV | SEQ ID NO.: 96 |
| CDH2 | 1 | Y40 | LCKTGFPEDVYSAVLSKDVHE | SEQ ID NO.: 97 |
| | 2 | Y70 | SNCNGKRKVQYESSEPADFKV | SEQ ID NO.: 98 |
| | 3 | Y87 | DFKVDEDGMVYAVRSFPLSSE | SEQ ID NO.: 99 |
| | 4 | Y104 | LSSEHAKFLIYAQDKETQEKW | SEQ ID NO.: |
| | | | | 100 |
| | 5 | Y194 | SDRDKNLSLRYSVTGPGADQP | SEQ ID NO.: |
| | | | | 101 |
| | 6 | Y284 | TVPEGSKPGTYVMTVTAIDAD | SEQ ID NO.: |
| | | | | 102 |
| | 7 | Y305 | DPNALNGMLRYRIVSQAPSTP | SEQ ID NO.: |
| | | | | 103 |
| | 8 | Y344 | AGLDREKVQQYTLIIQATDME | SEQ ID NO.: |
| | | | | 104 |
| | 9 | Y359 | QATDMEGNPTYGLSNTATAVI | SEQ ID NO.: |
| | | | | 105 |
| | 10 | Y387 | NPPEFTAMTFYGEVPENRVDI | SEQ ID NO.: |
| | | | | 106 |
| | 11 | Y419 | QPHTPAWNAVYRISGGDPTGR | SEQ ID NO.: |
| | | | | 107 |
| | 12 | Y495 | VTVIDVNENPYFAPNPKIIRQ | SEQ ID NO.: |
| | | | | 108 |
| | 13 | Y526/Y533 | TTFTAQDPDRYMQQNIRYTKL | SEQ ID NO.: |
| | | | | 109 |
| | 14 | Y526/Y533 | PDRYMQQNIRYTKLSDPANWL | SEQ ID NO.: |
| | | | | 110 |
| | 15 | Y570 | RESPNVKNNIYNATFLASDNG | SEQ ID NO.: |
| | | | | 111 |
| | 16 | Y593 | PMSGTGTLQIYLLDINDNAPQ | SEQ ID NO.: |
| | | | | 112 |
| | 17 | Y627 | PNSINITALDYDIDPNAGPFA | SEQ ID NO.: |
| | | | | 113 |
| | 18 | Y675 | LKIKFLEAGIYEVPIIITDSG | SEQ ID NO.: |
| | | | | 114 |

Immunostaining and immunofluorescence staining. HUH7 or HUH7ko or HEP3B or HEP3B-MAN2A1-FER cells were fixed with 4% paraformaldehyde for 15 minutes. These cells were blocked with 10% goat serum, 0.4% triton x-100 in PBS for one hour, followed by incubation with 2-3B-G8, 1-3C-F11, 2-3C-C5, 2-6B-D8, 1-4B-B10, or IgG antibody (100 ng/ml) in 2% goat serum in PBS for one hour. The cells were then incubated with FITC-conjugated goat anti-mouse antibody in 2% goat serum in PBS for an hour, followed by a DAPI incubation for 10 minutes. The imaging was taken with a fluorescence microscope (Olympus) or assayed for flow cytometry (BD FACSCalibur).

Xenografted tumor growth and treatment. Male severe combined immunodeficiency (SCID) mice were used. Approximately 5×10⁶ viable HepG2, HUH7, and HUH7/ko cells suspended in 0.2 mL of Hanks' balanced salt solution (Krackeler Scientific, Inc., Albany, NY) were subcutaneously implanted in the abdominal flanks of severe combined immunodeficient mice (SCID) mice to generate one tumor per mouse. Two weeks after xenografting of HEPG2, the tumors reached an average size of 752 mm³. These mice were treated with 2-3B-G8-MMAE or IgG-MMAE (4 ng/kg), 2 times a week through tail vein applications. After 5 weeks, all survived mice were killed, and necropsies were performed. For mice treated with control IgG, necropsies were performed when mice died from the xenografted cancers. For HUH7 or HUH7ko xenografted mice, the treatment started when the tumor reached the average sizes of 117 mm³ or 107 mm³ (10 days), respectively. Similar treatment schemes as above were applied to these animals. All HUH7 control-treated animals died within two weeks of the treatment. The treatment and non-treatment groups were randomized and blinded to the researchers.

### Results

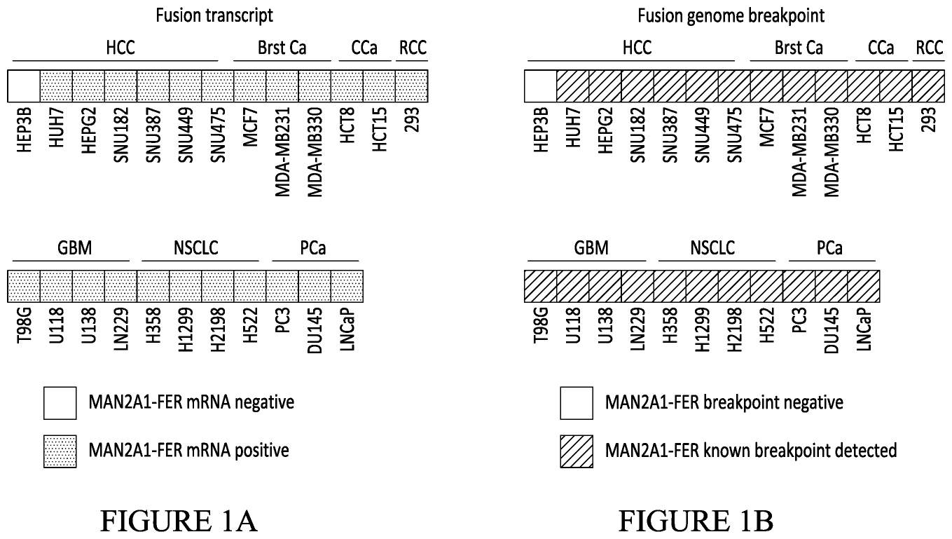

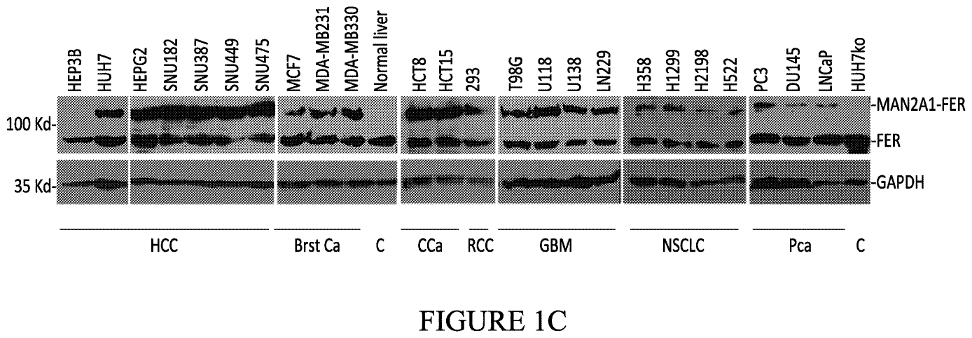

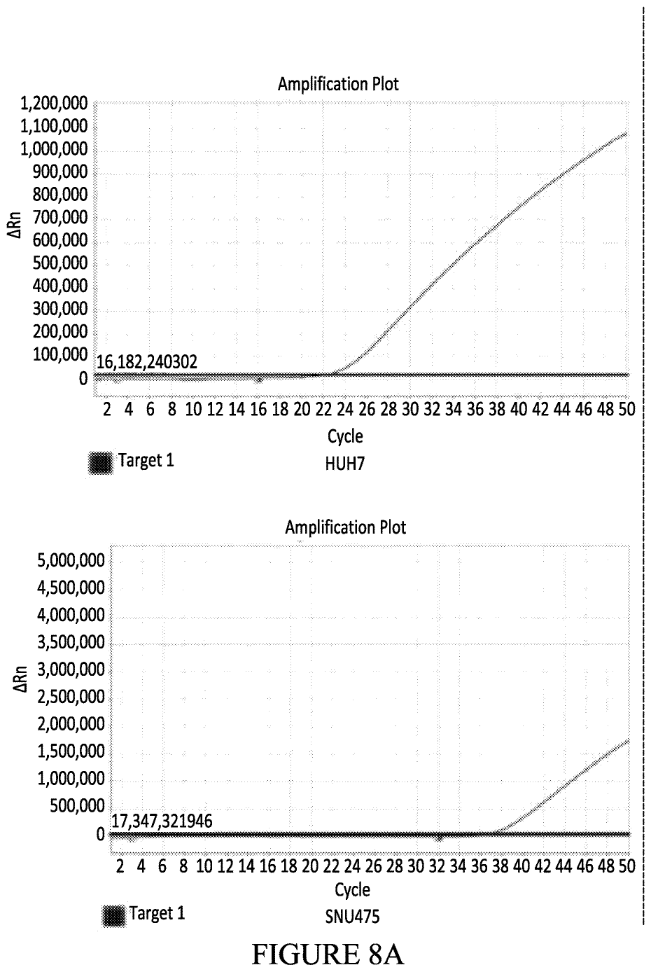

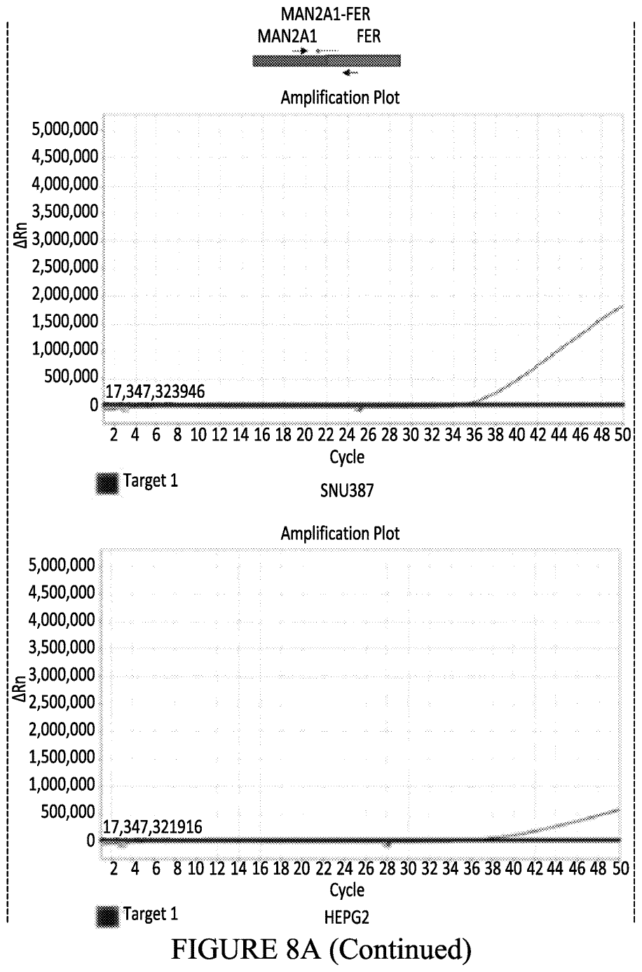

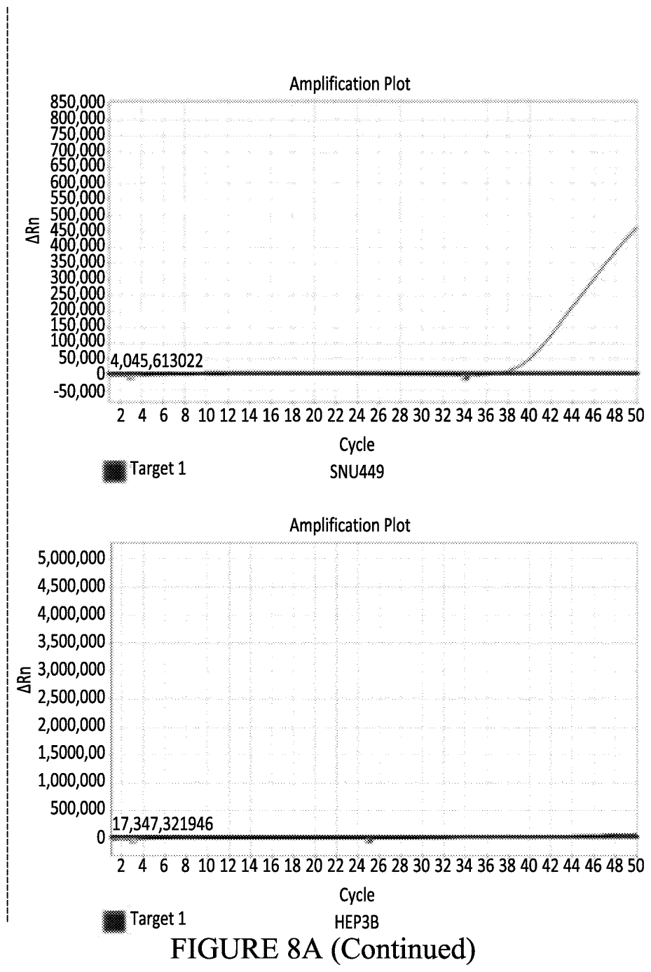

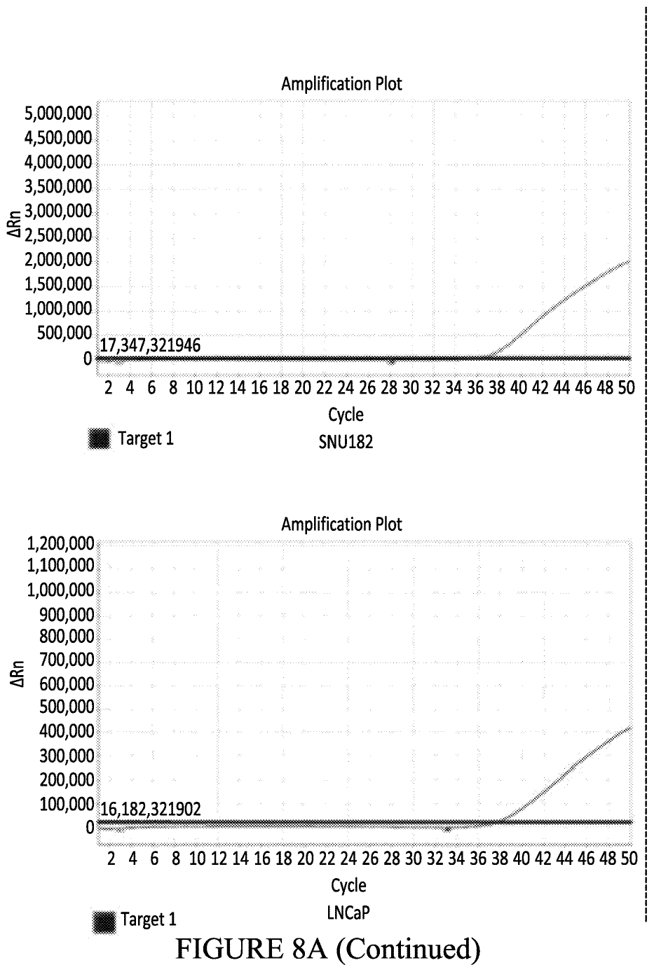

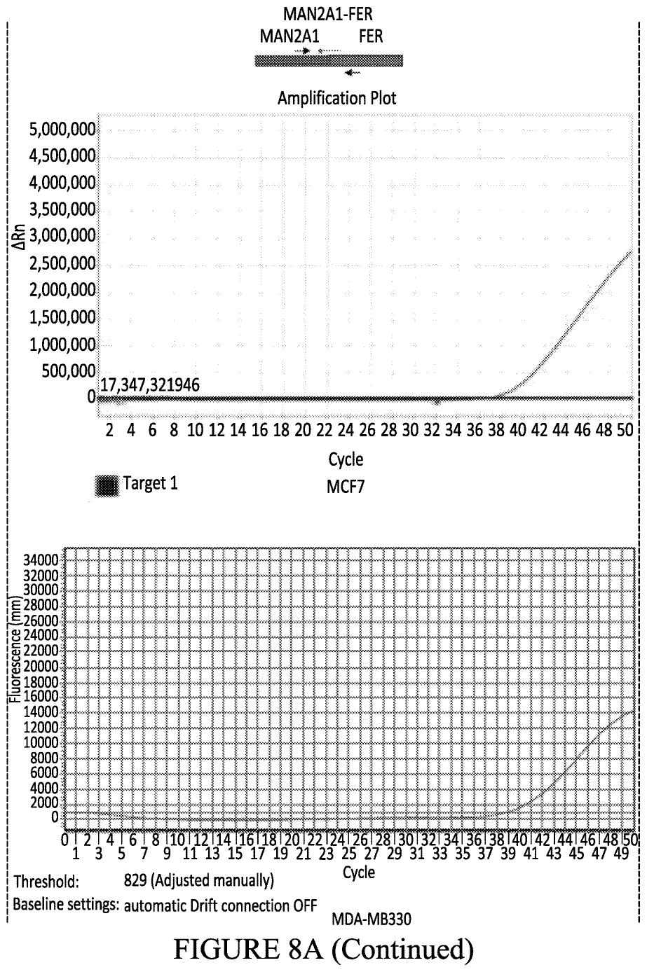

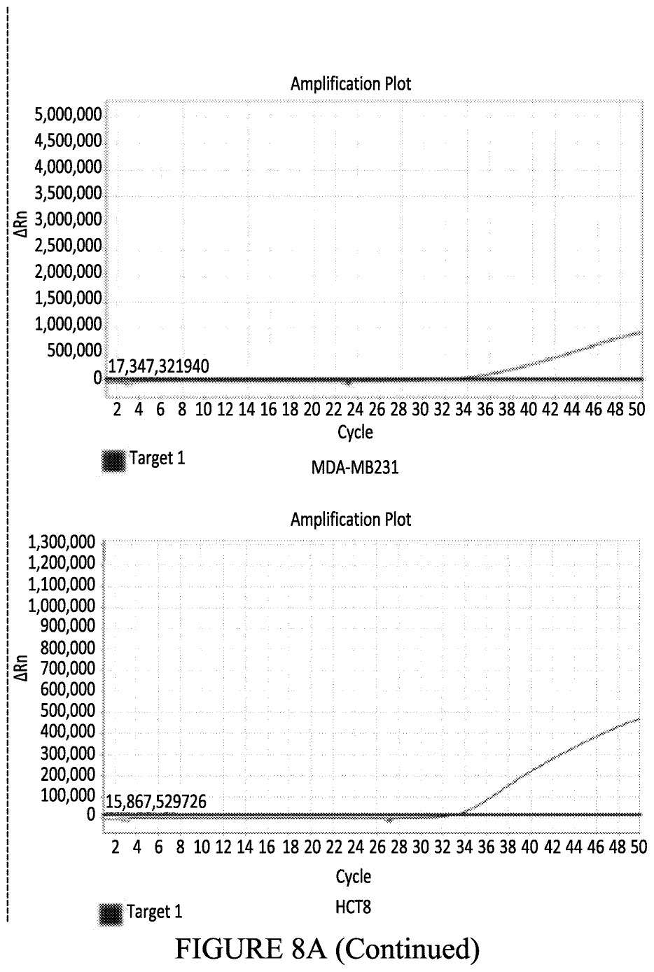

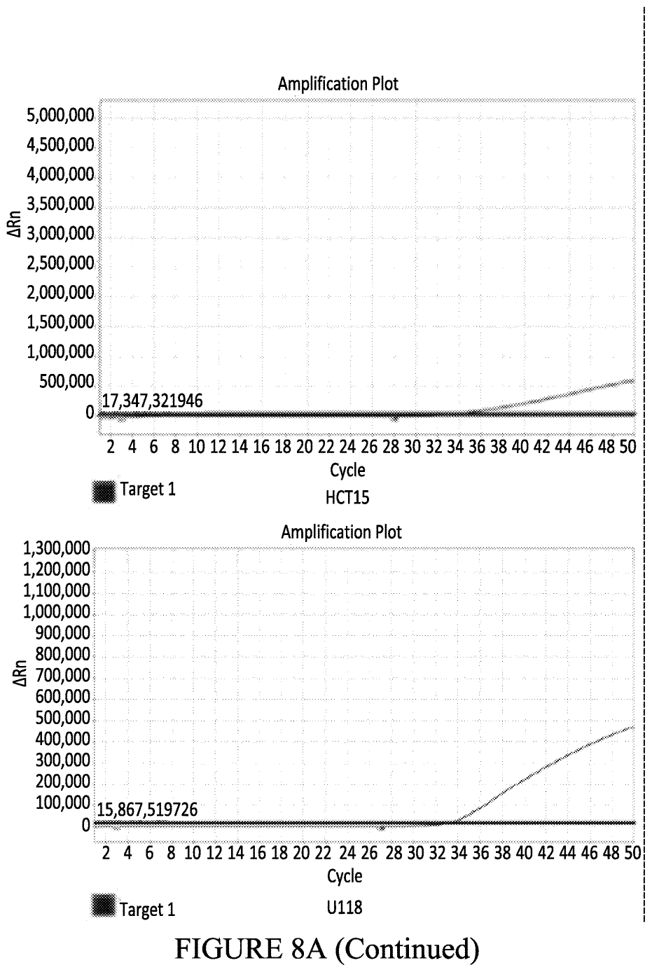

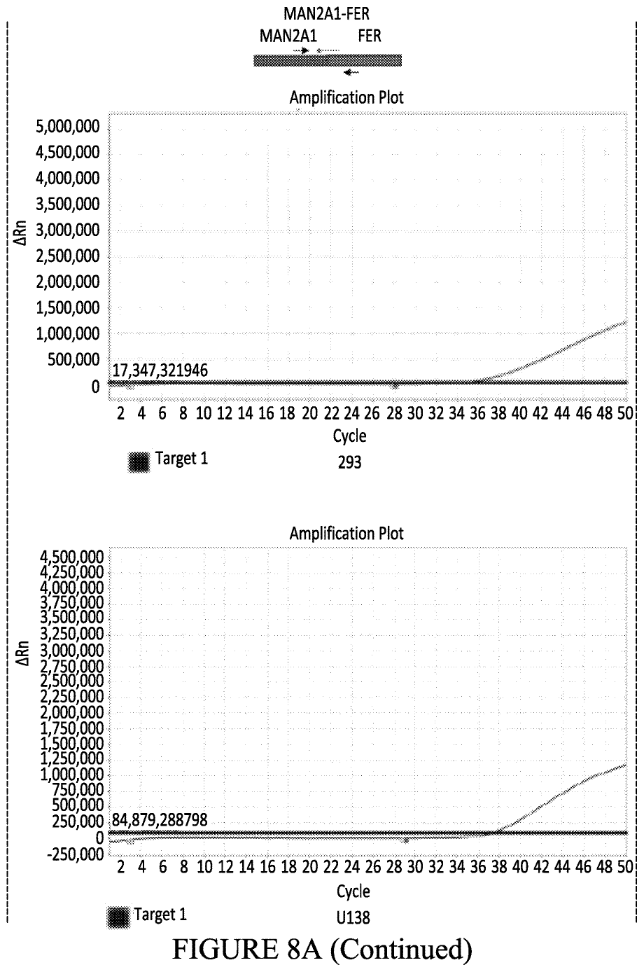

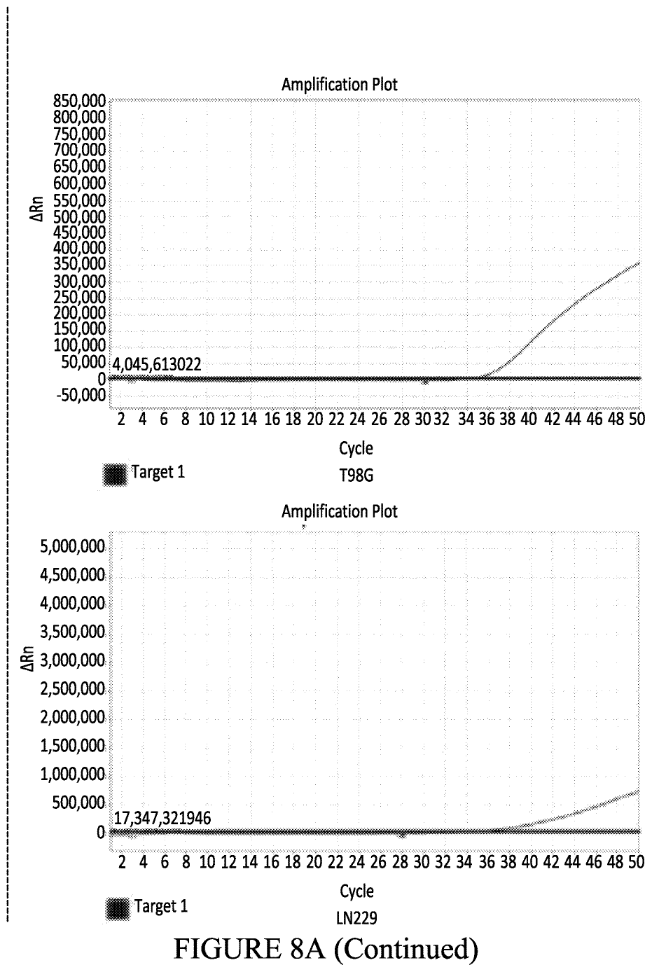

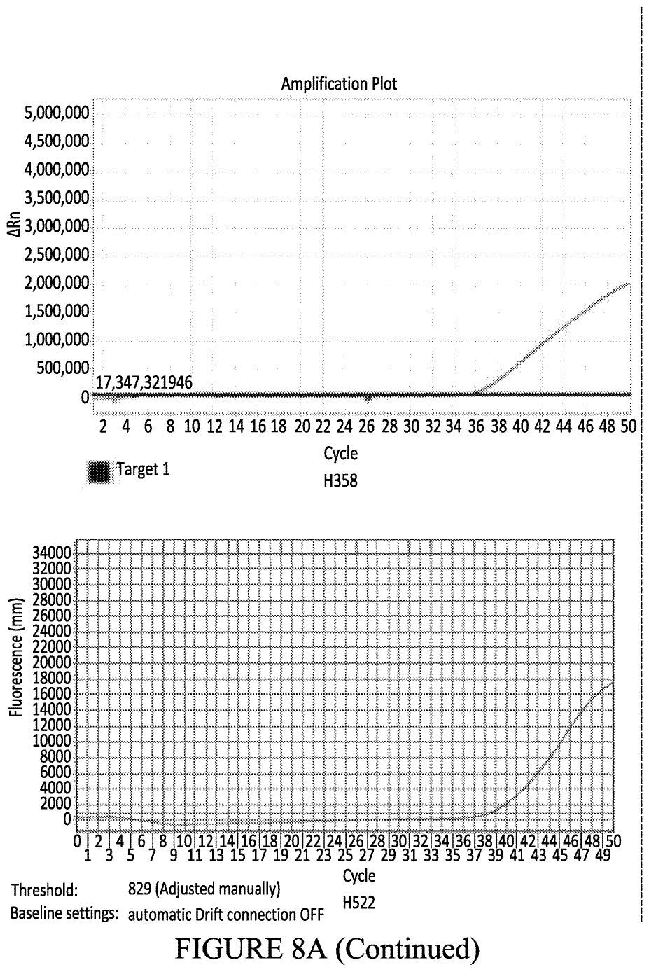

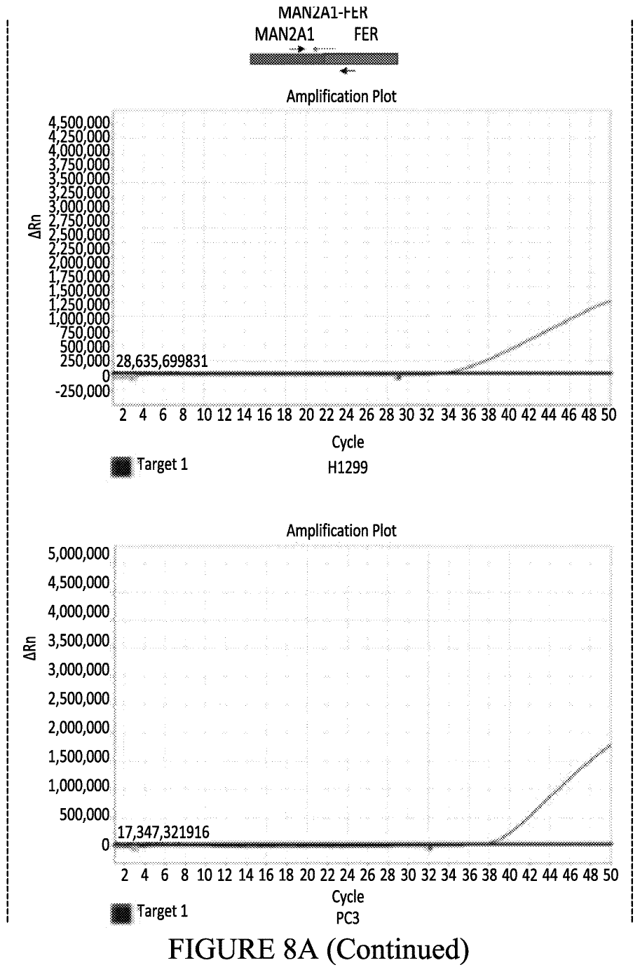

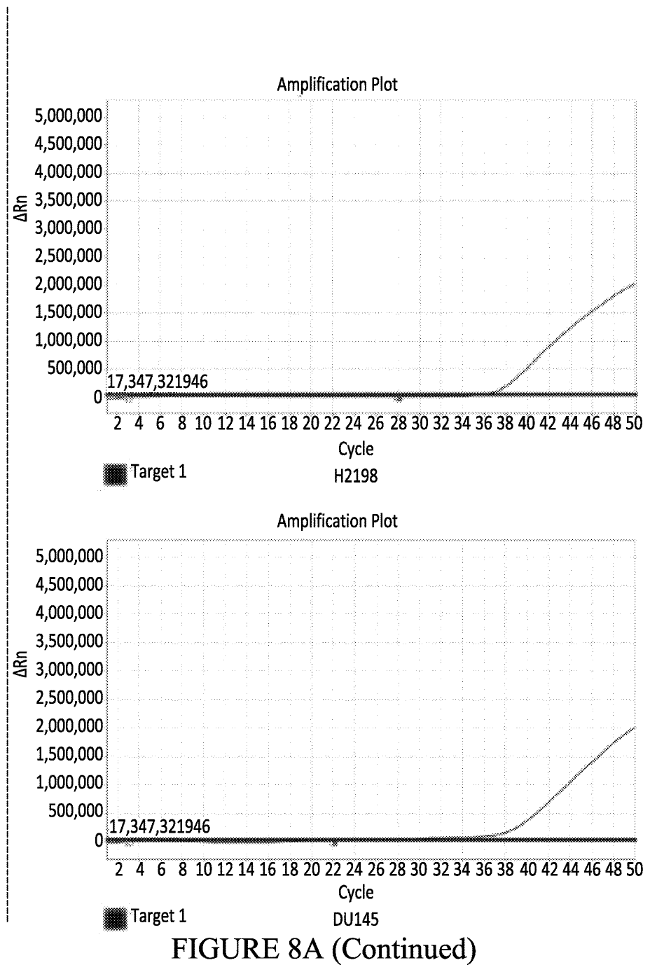

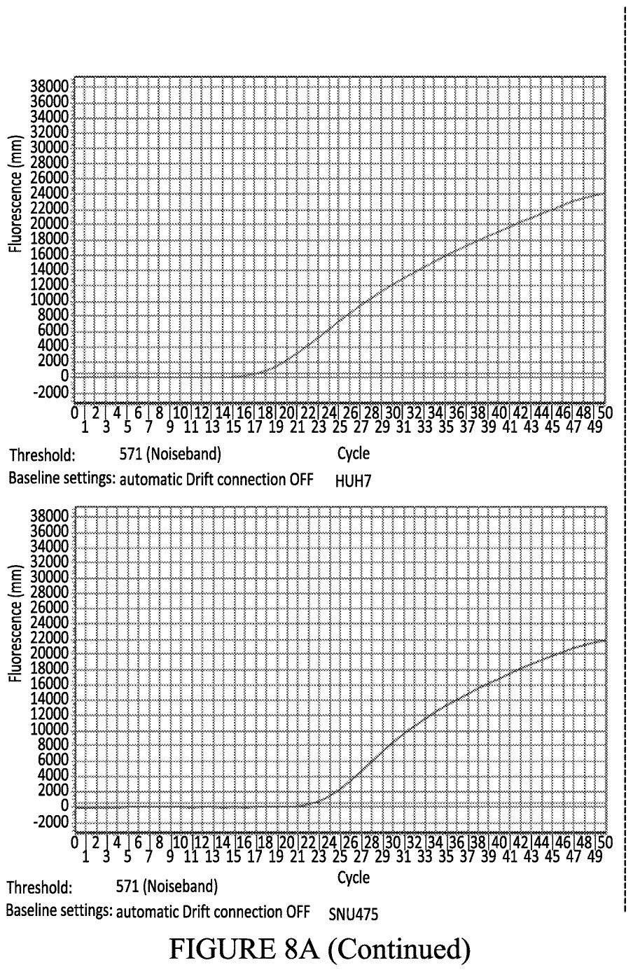

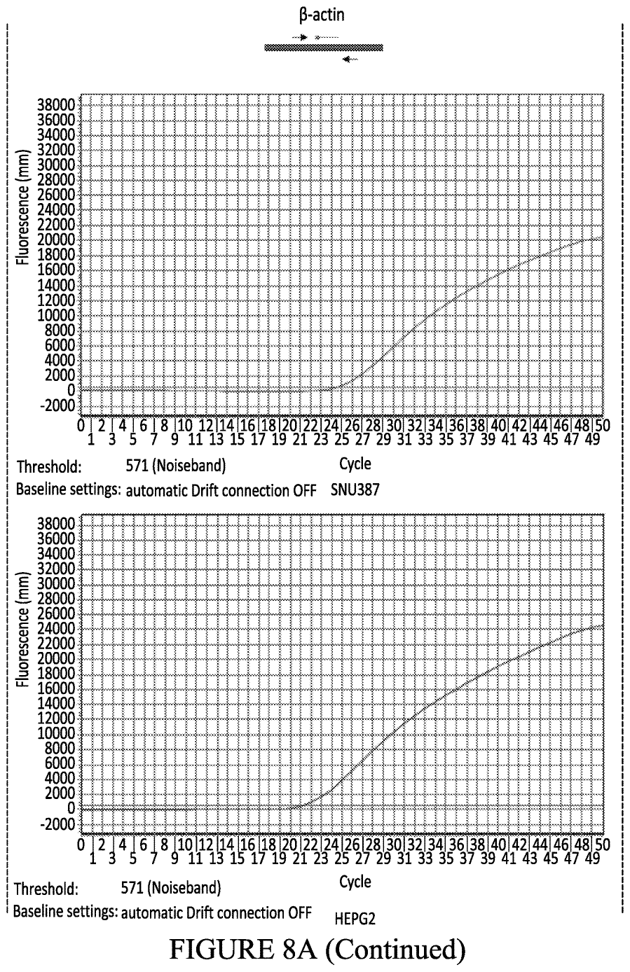

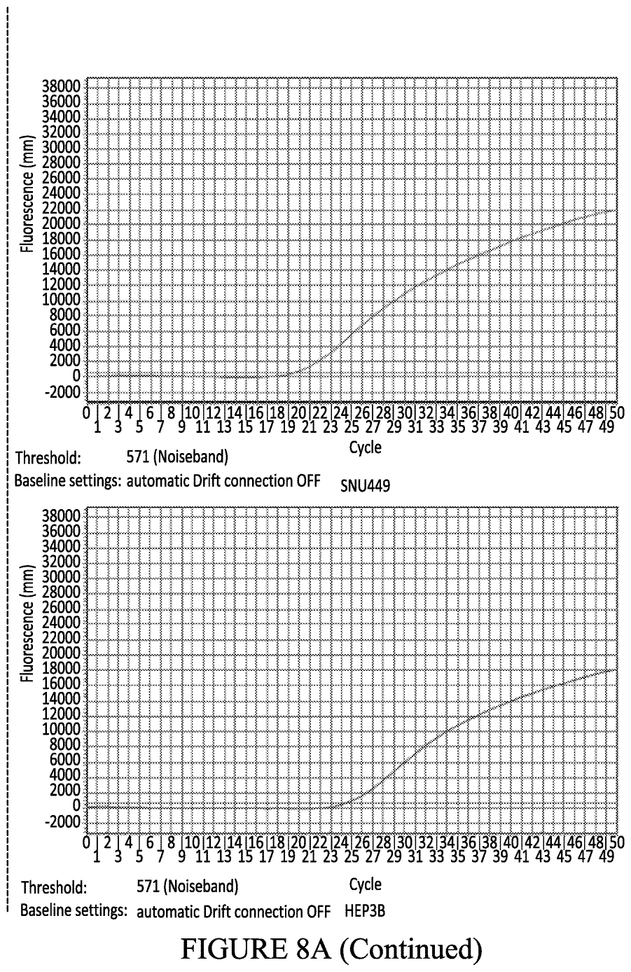

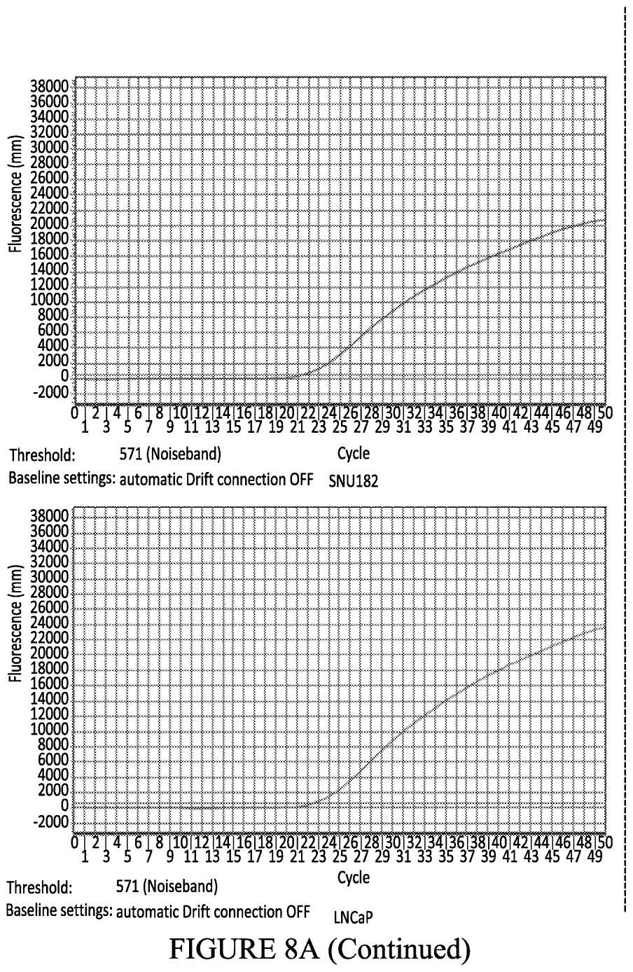

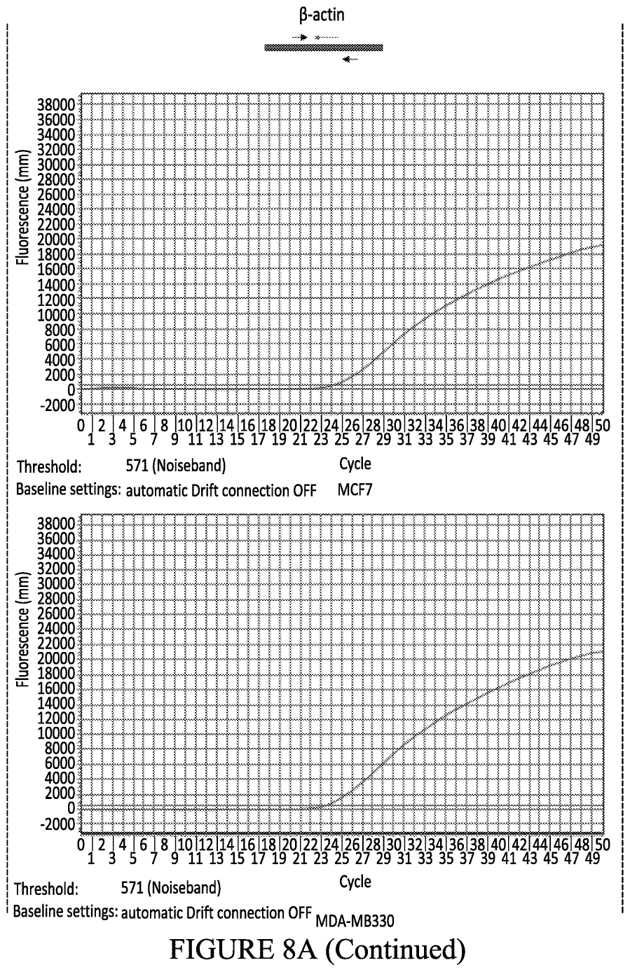

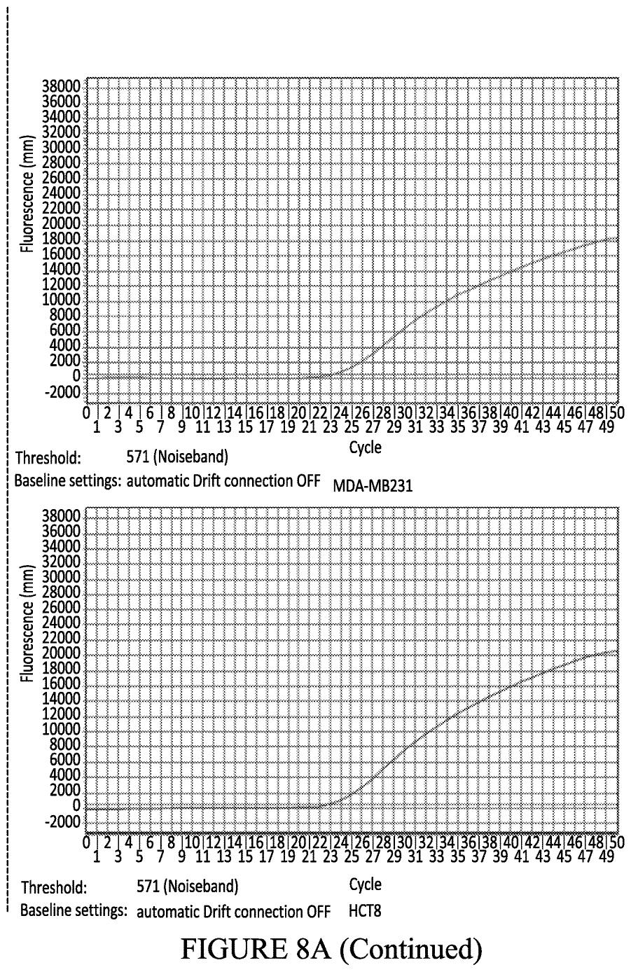

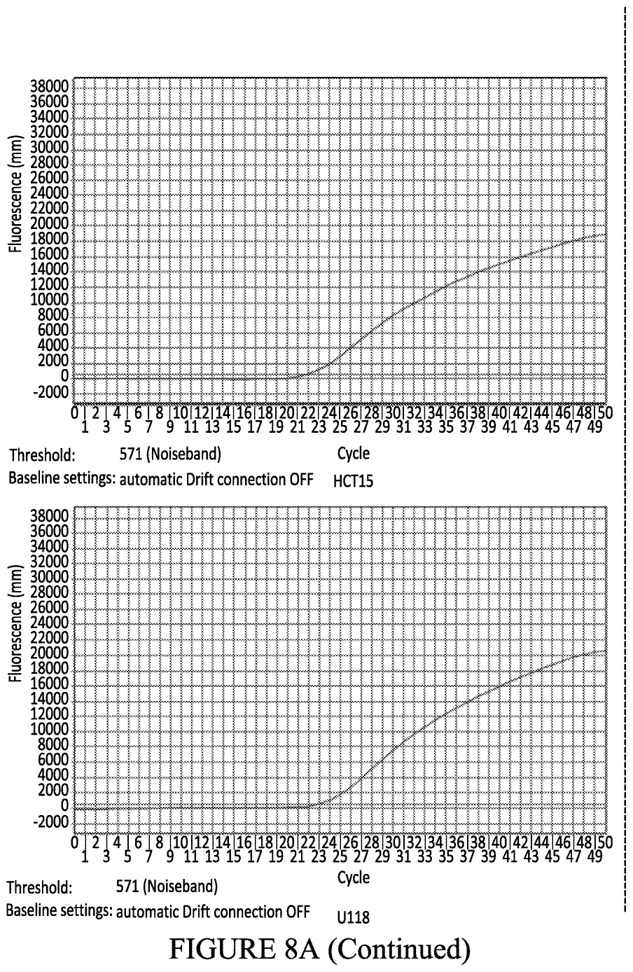

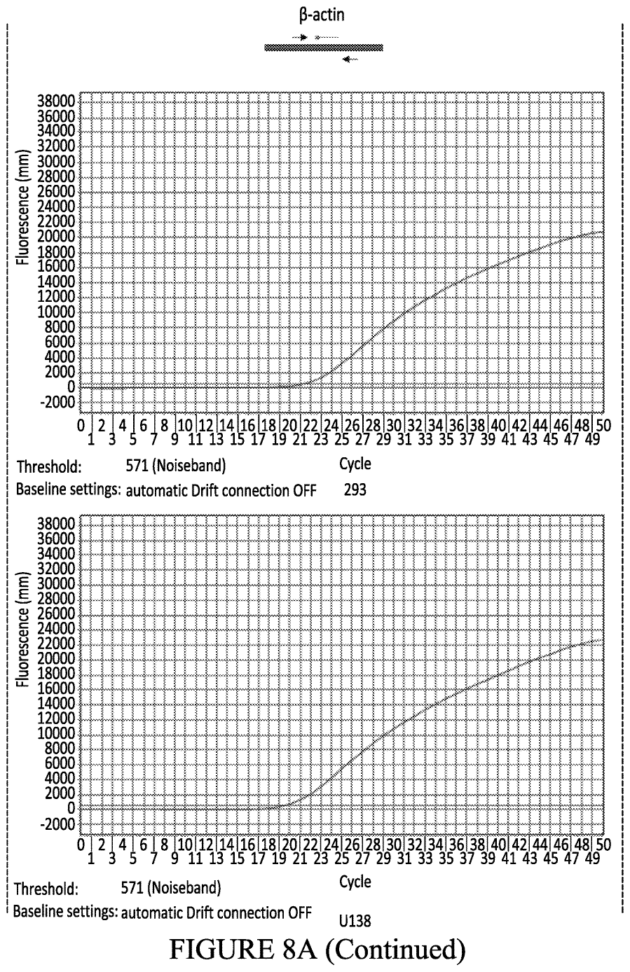

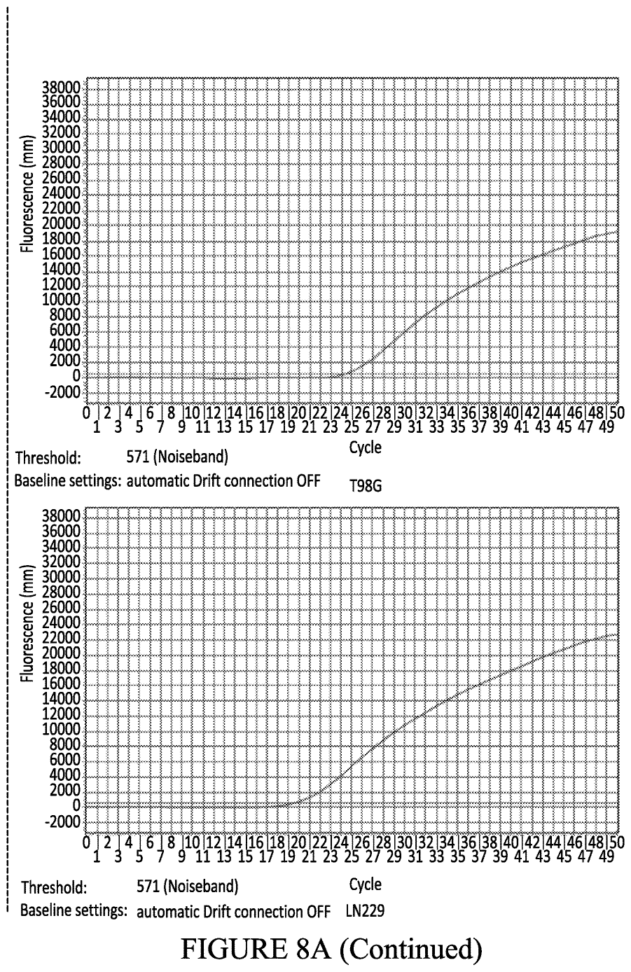

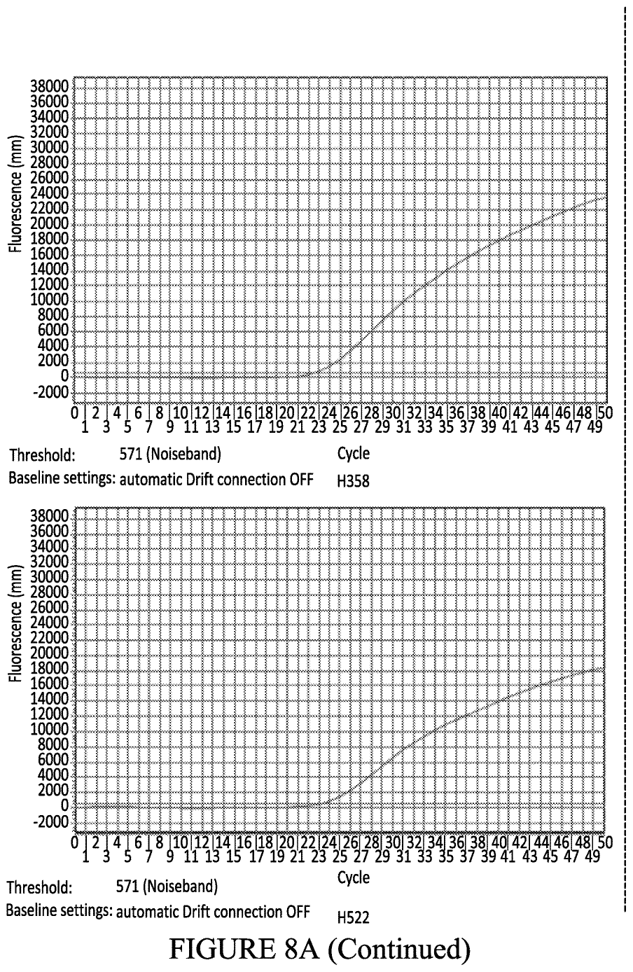

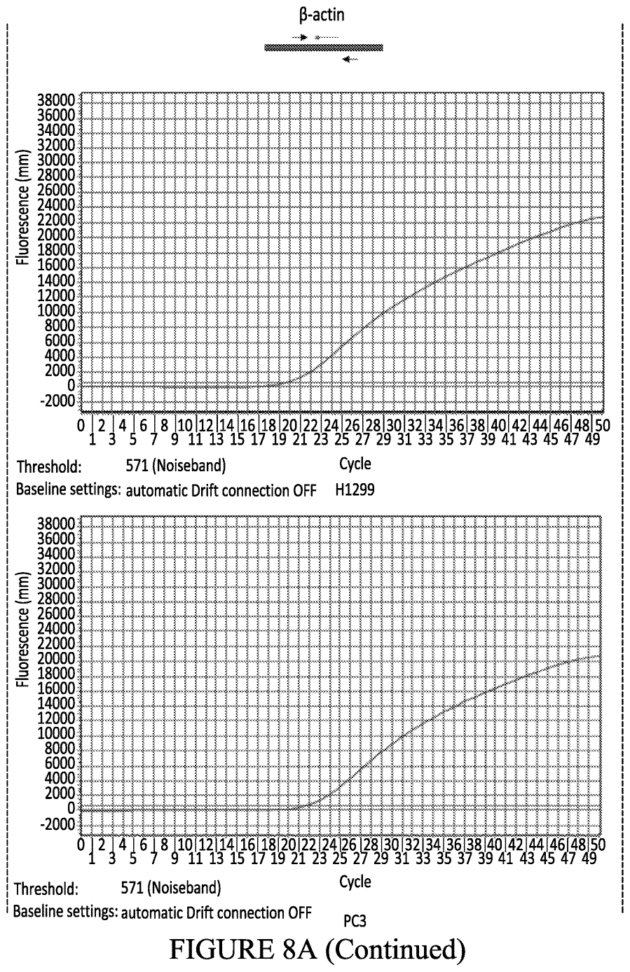

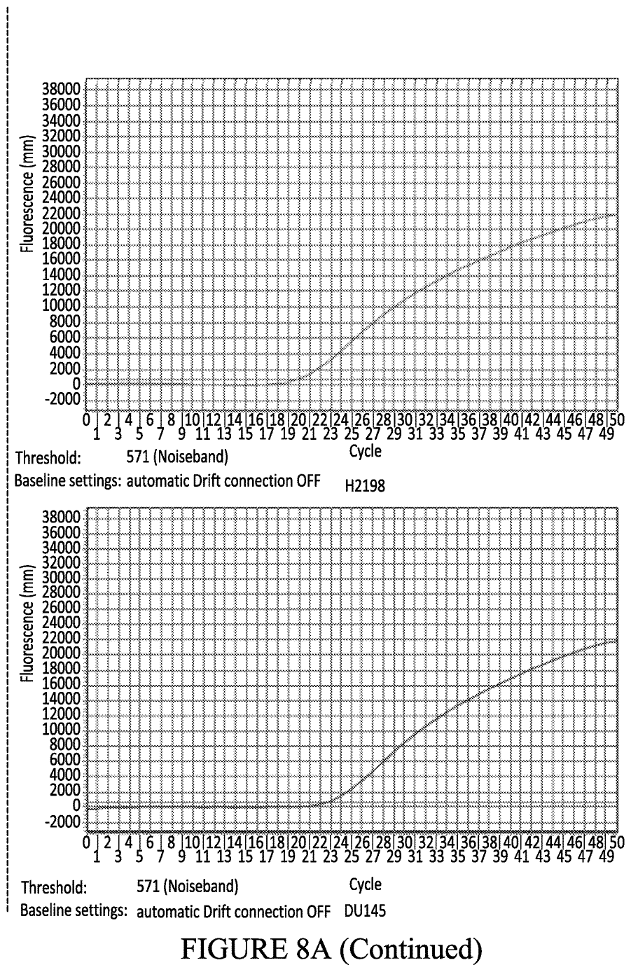

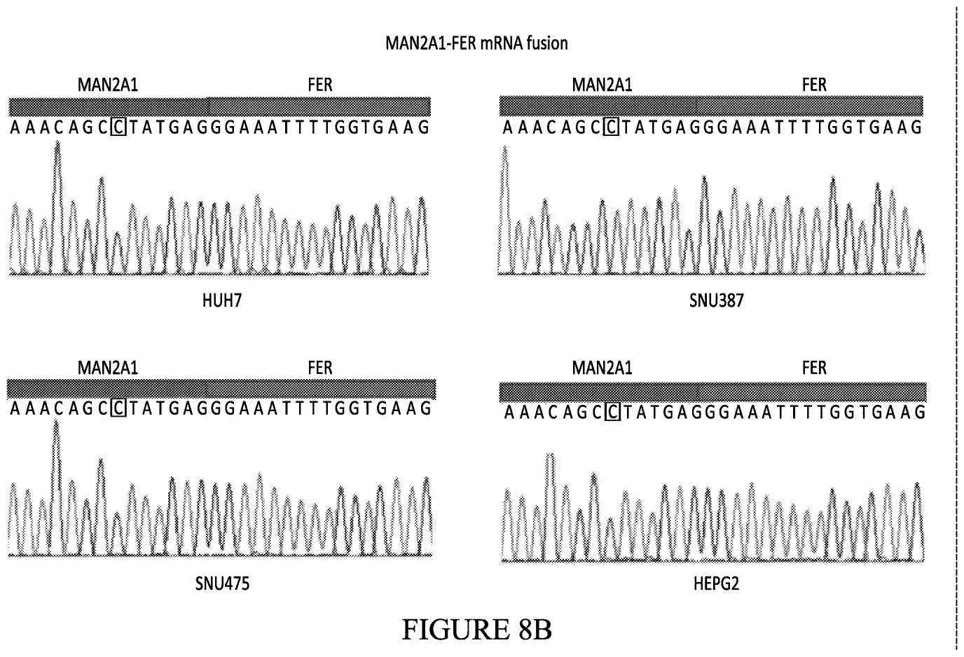

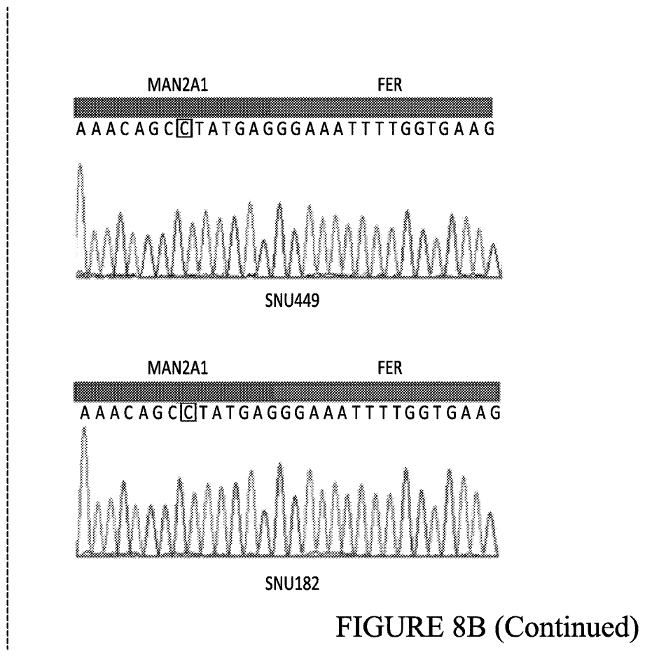

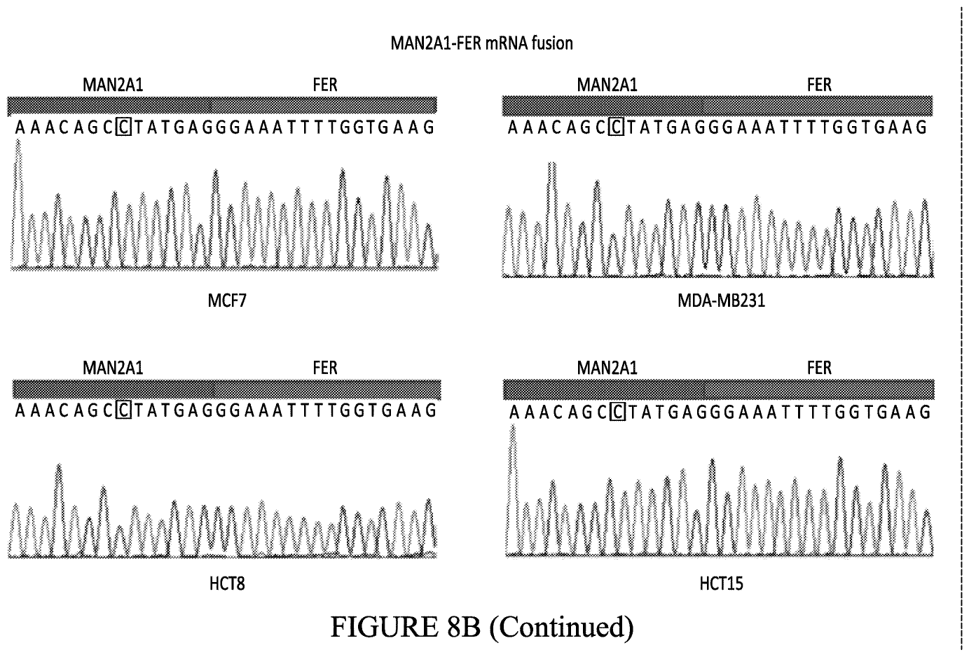

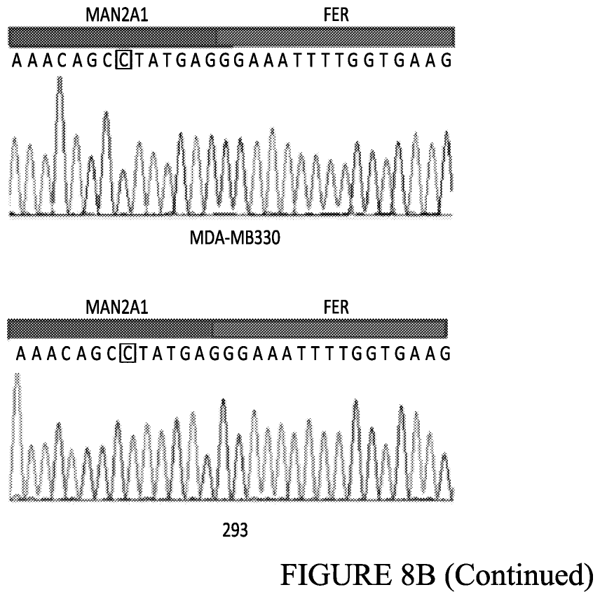

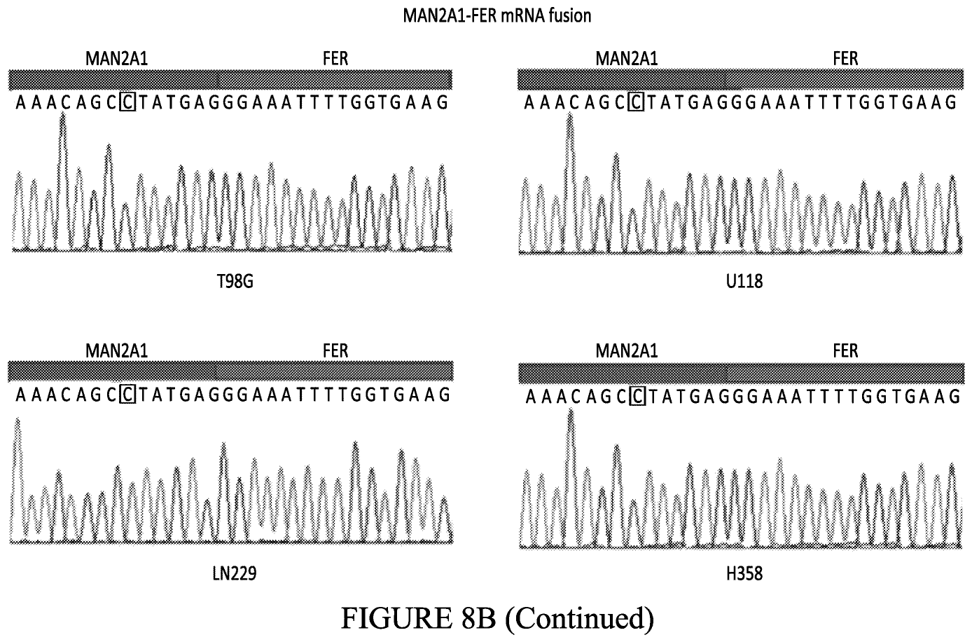

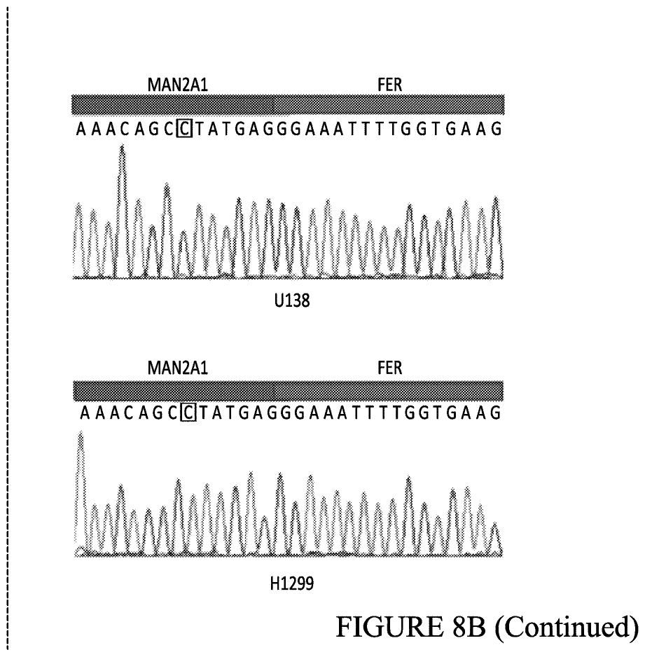

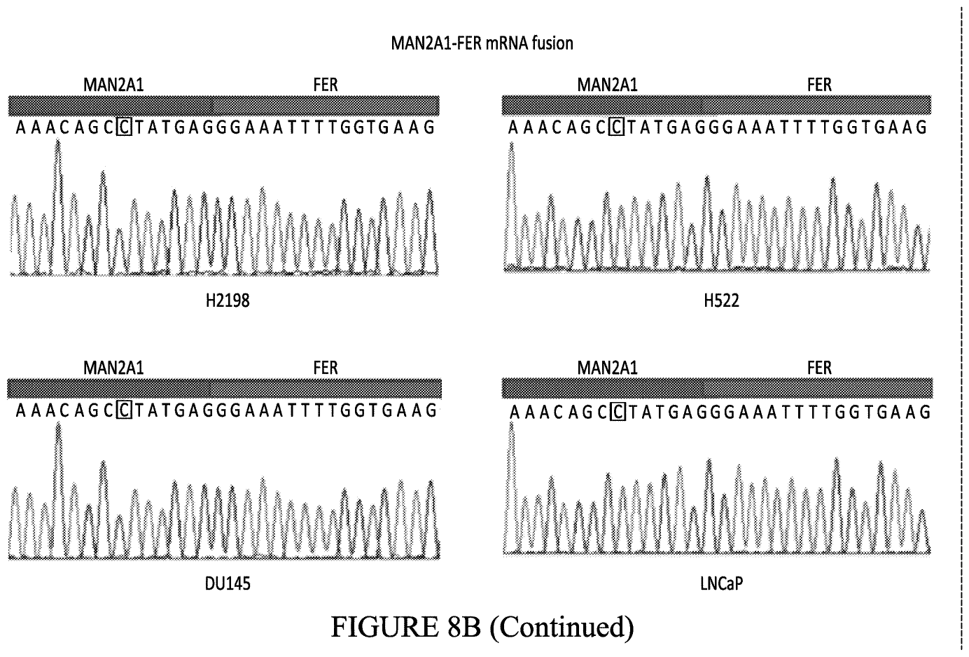

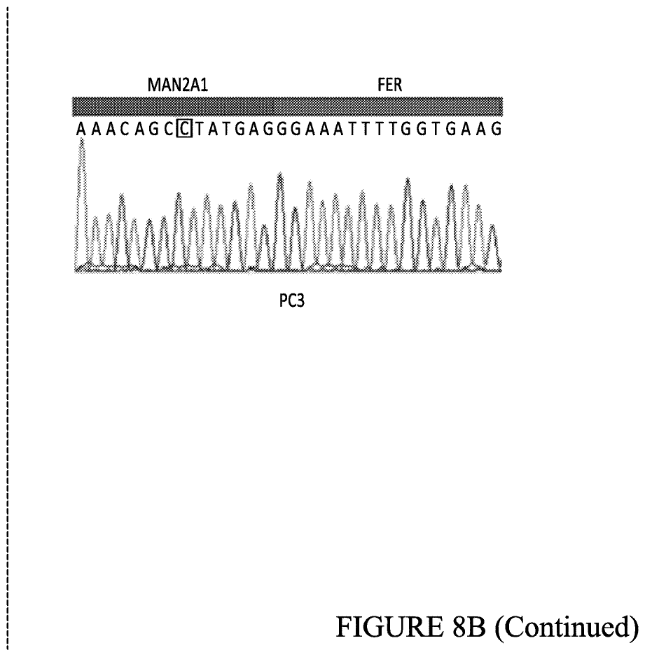

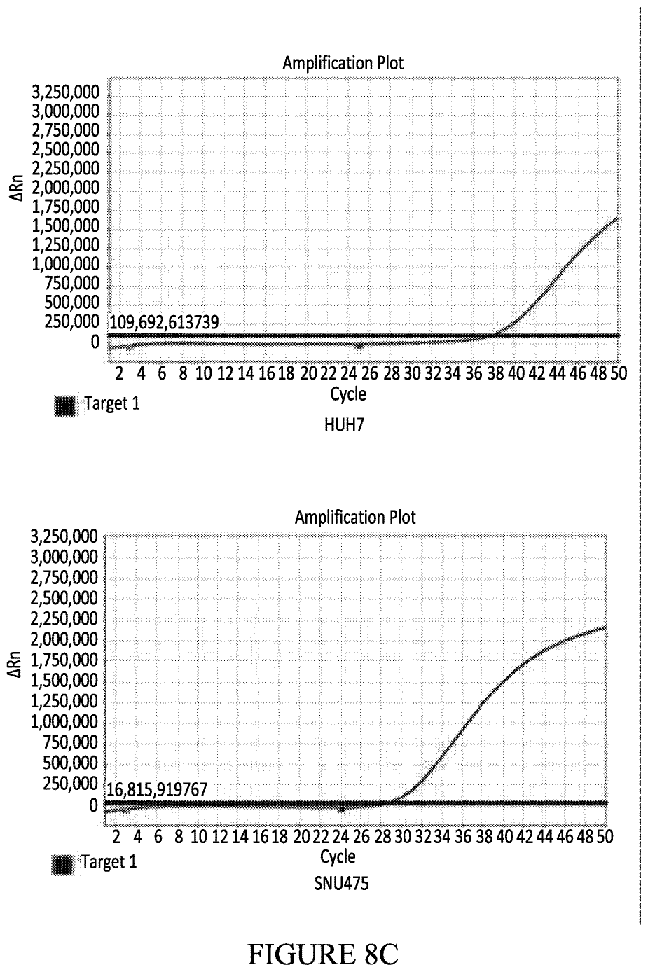

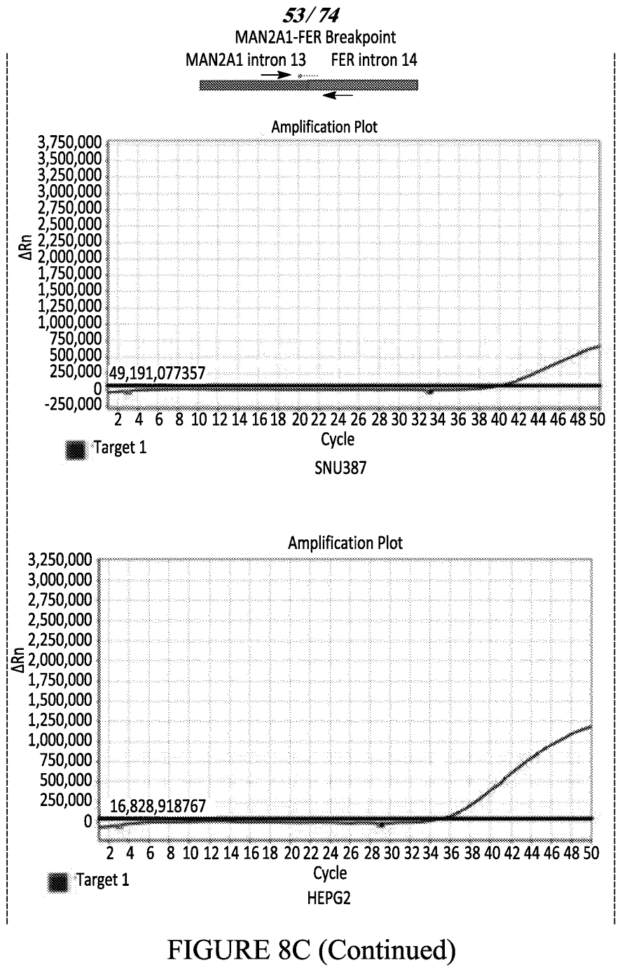

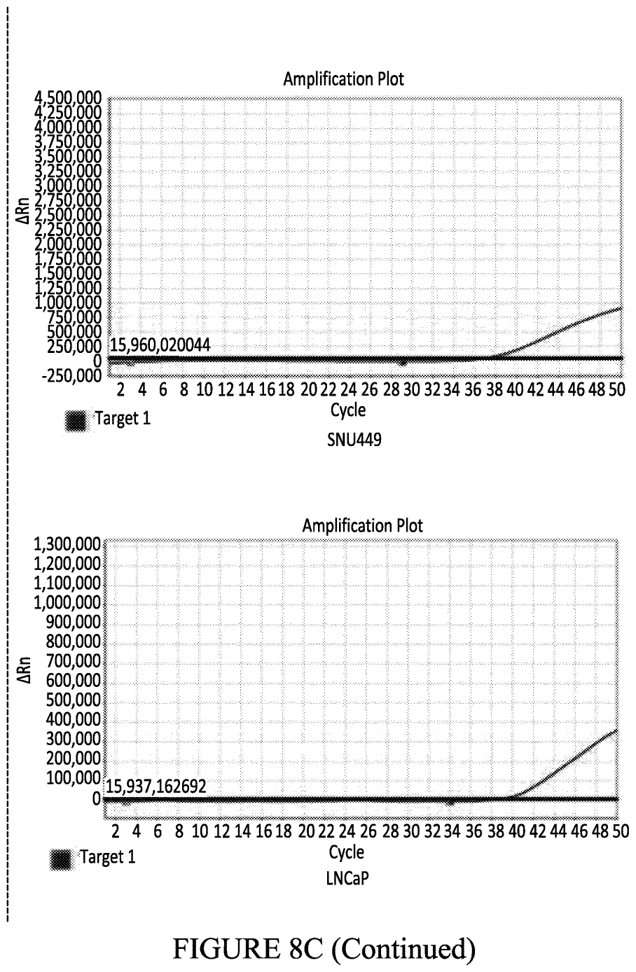

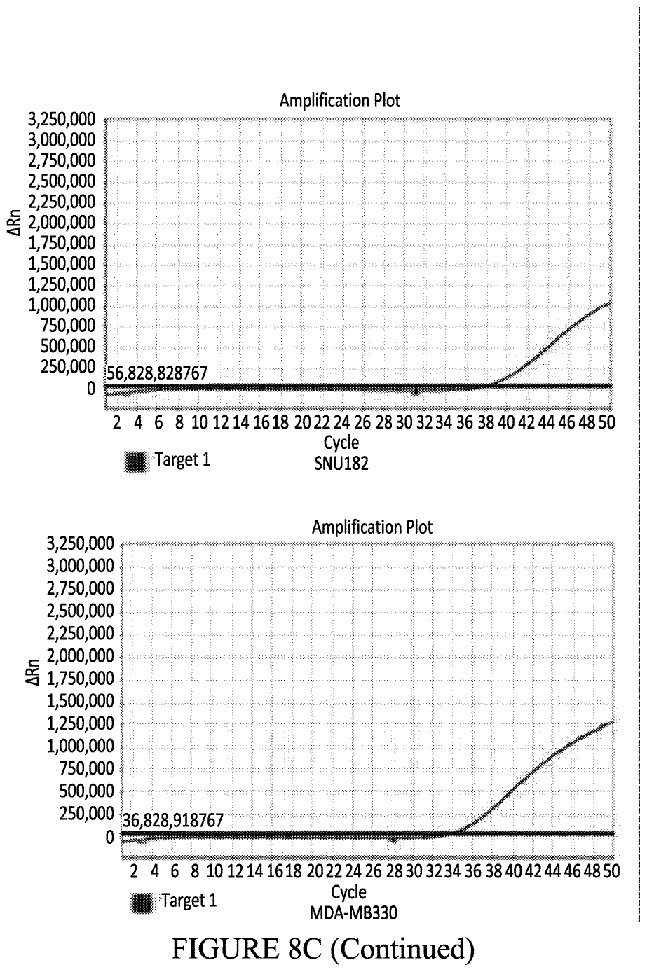

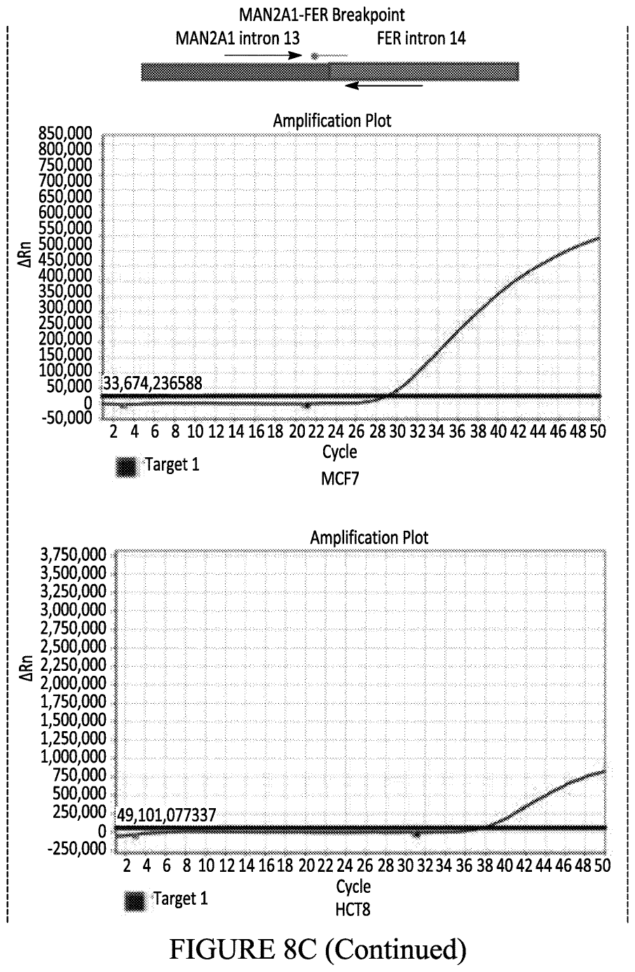

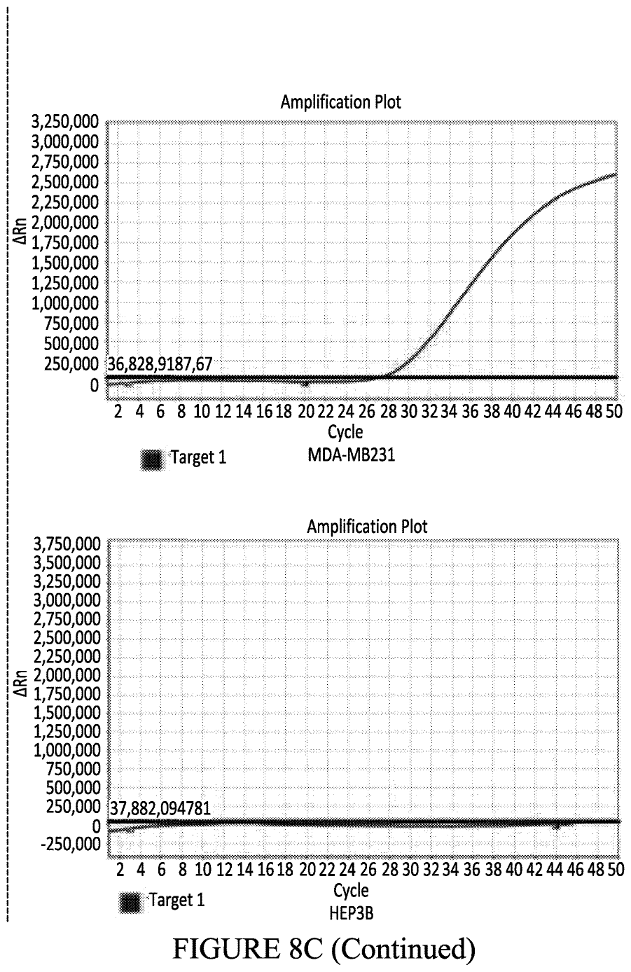

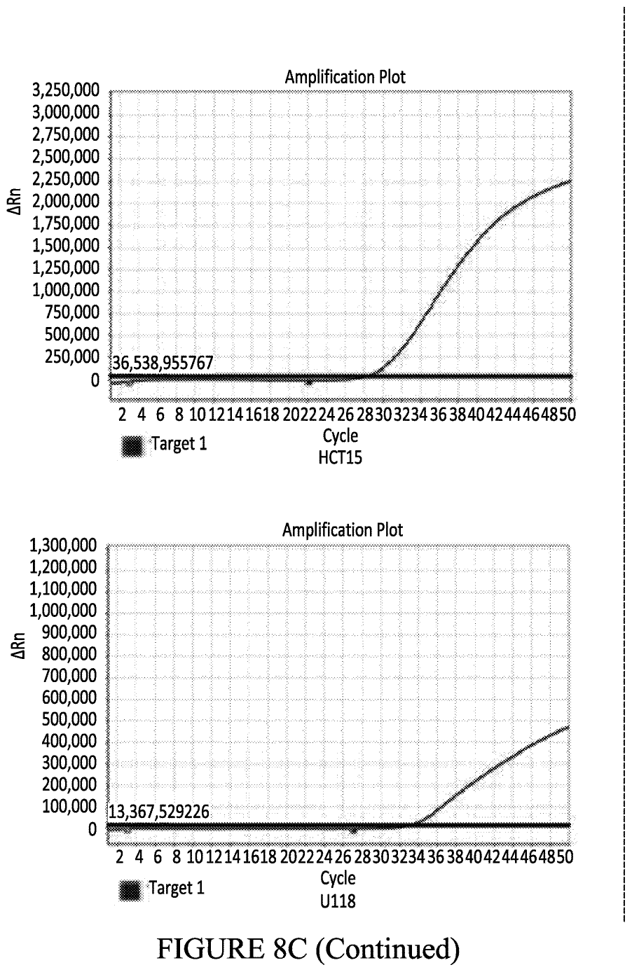

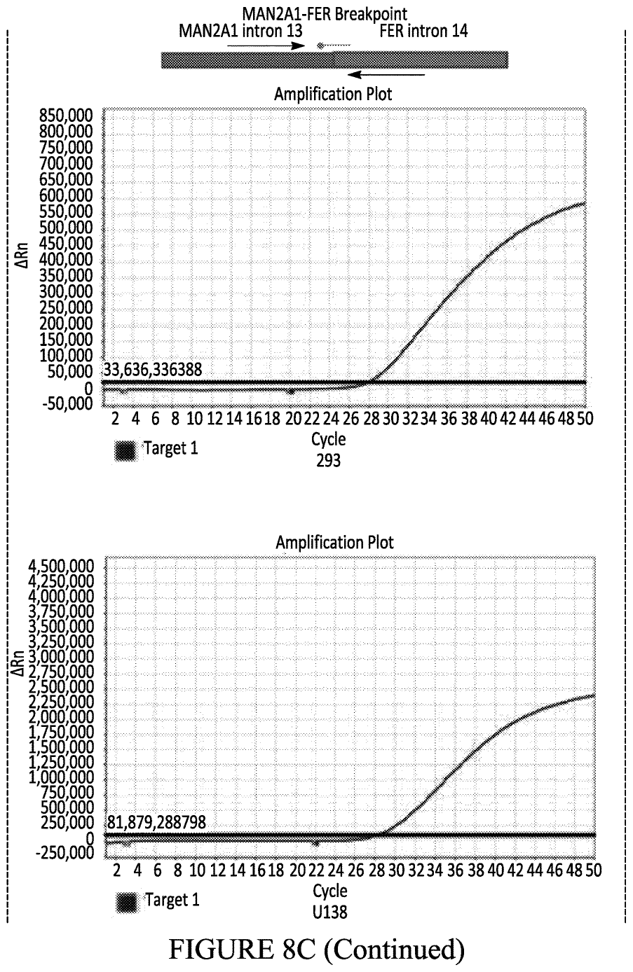

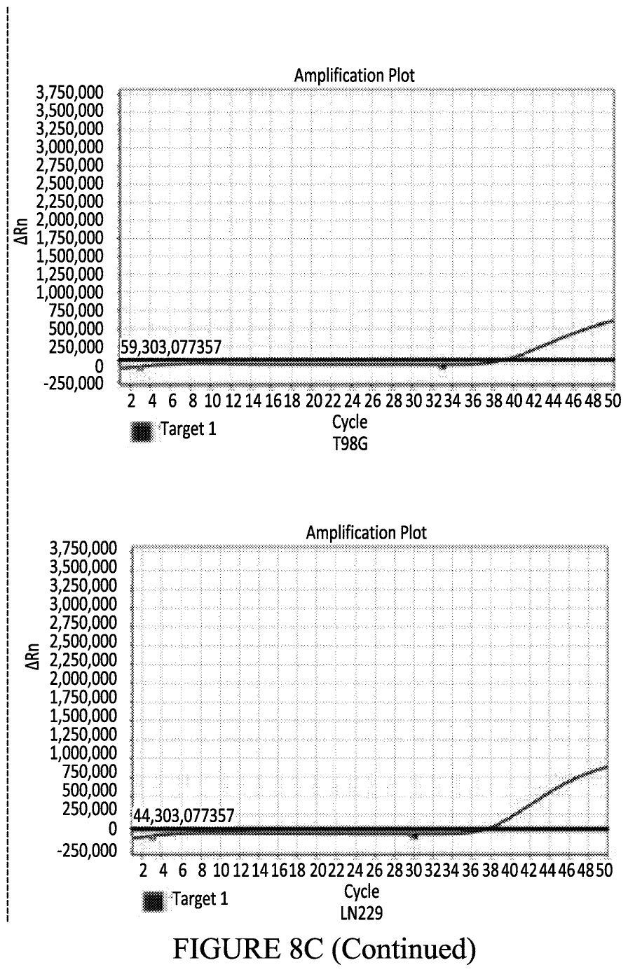

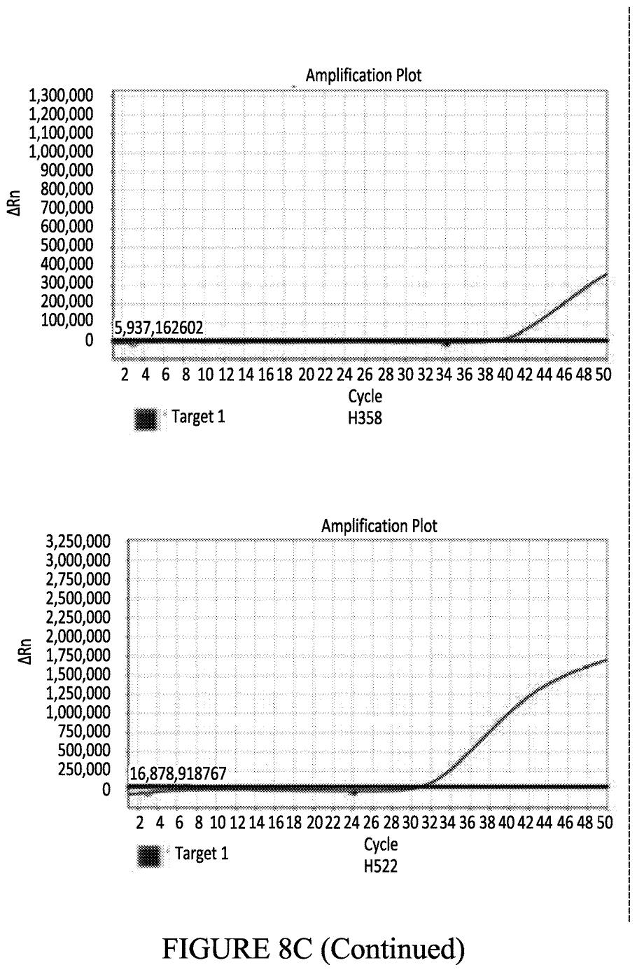

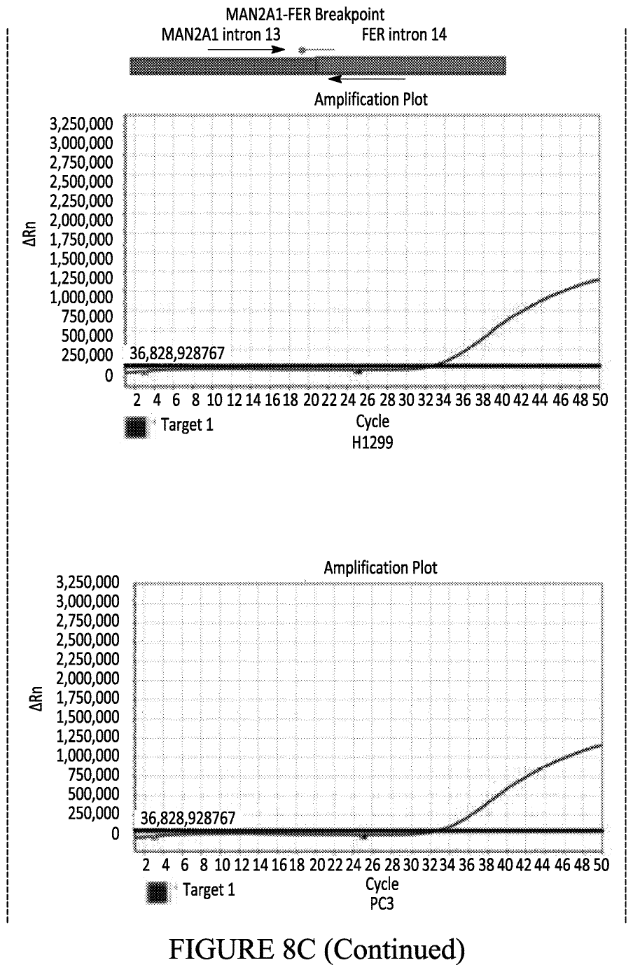

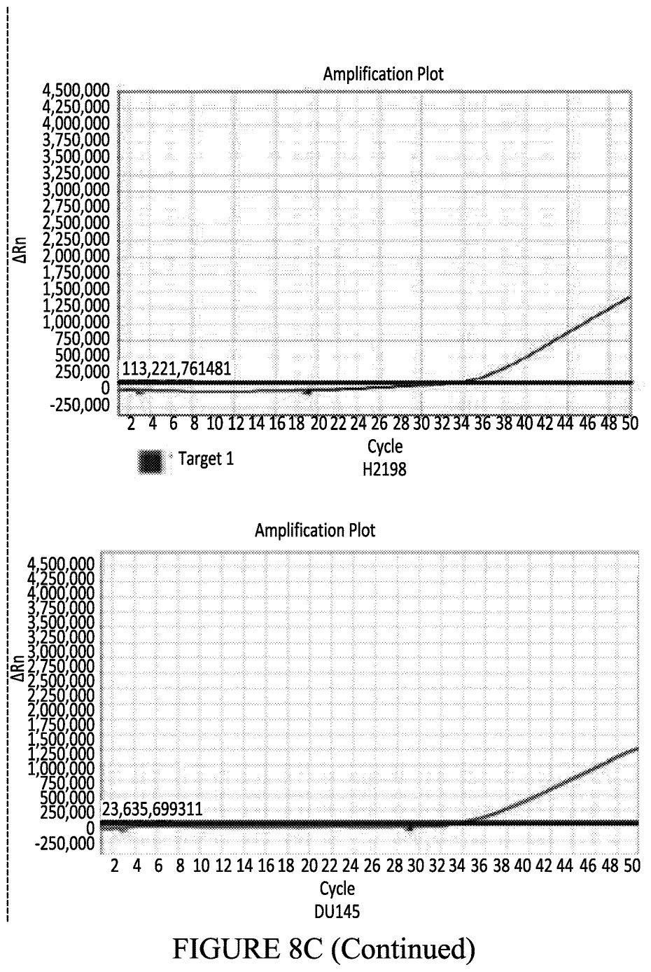

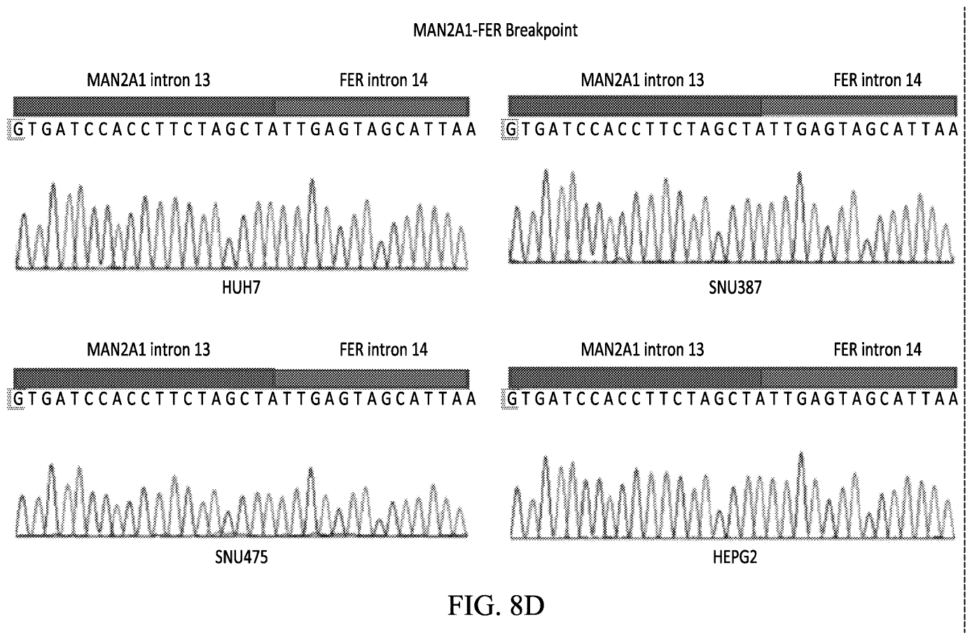

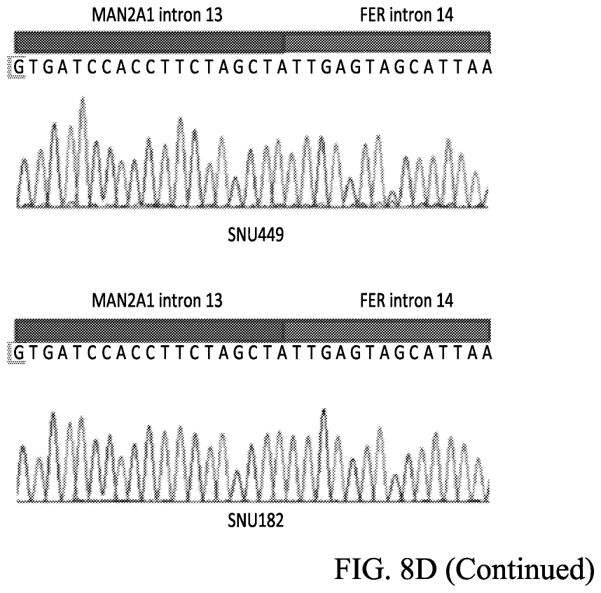

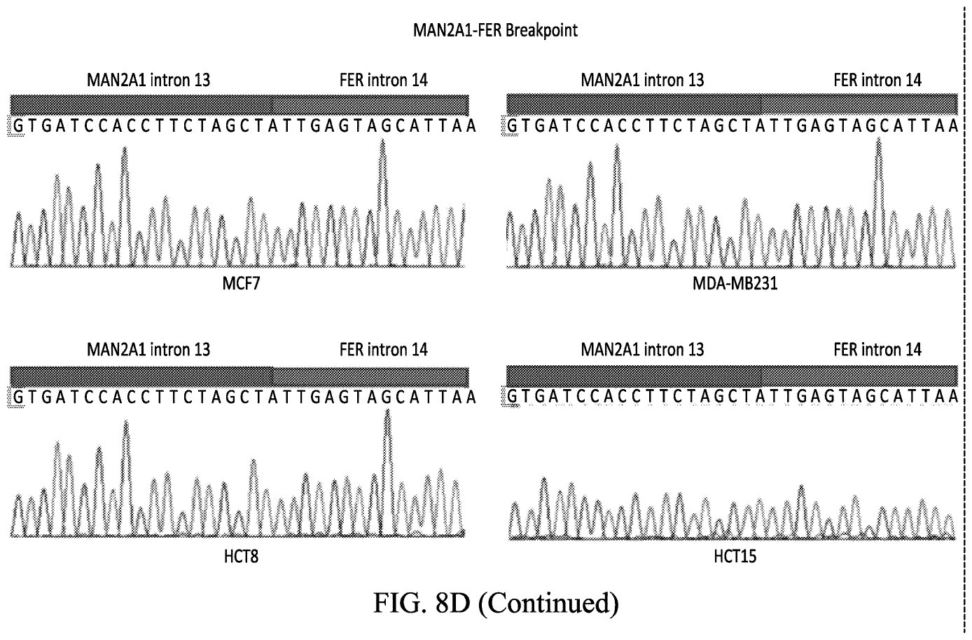

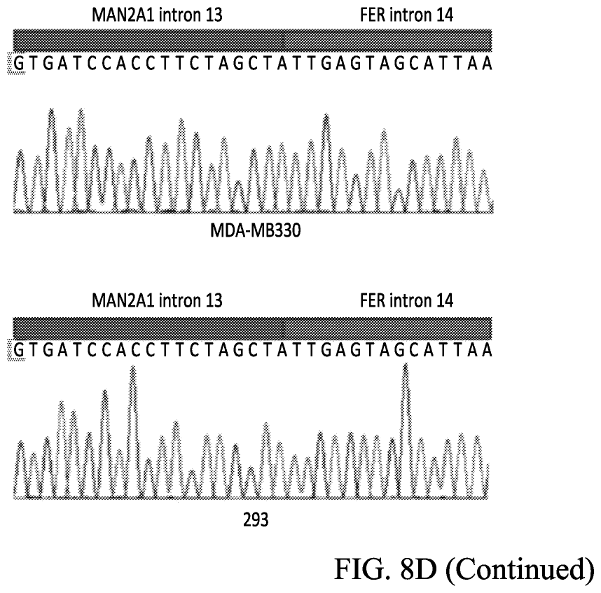

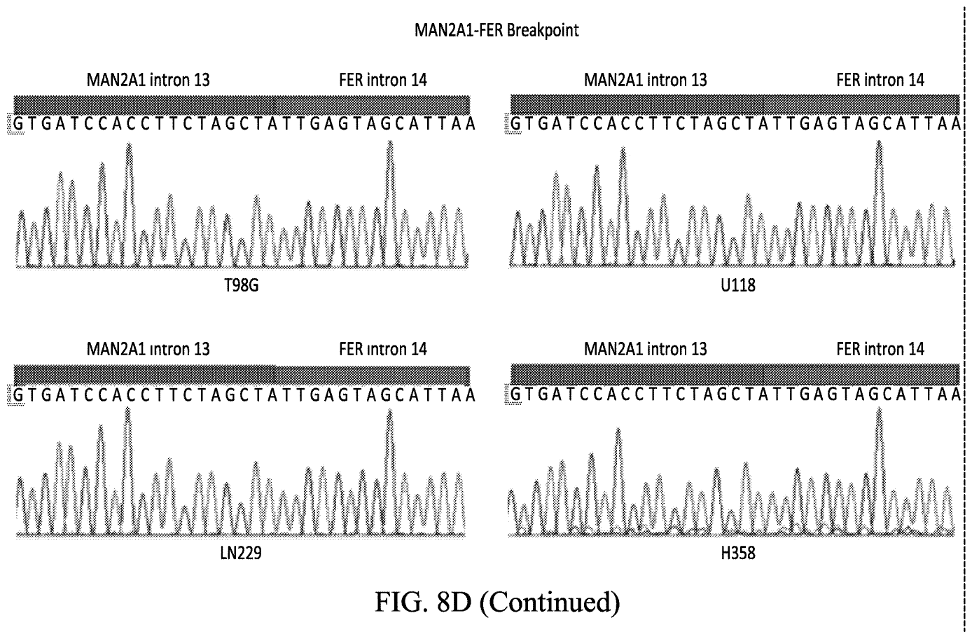

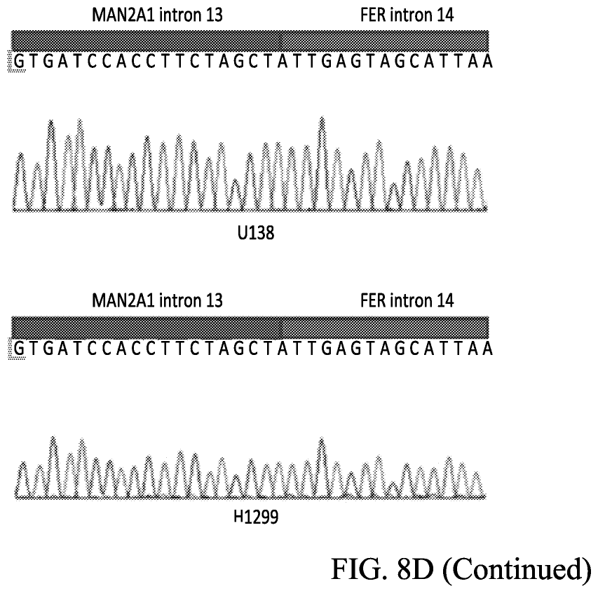

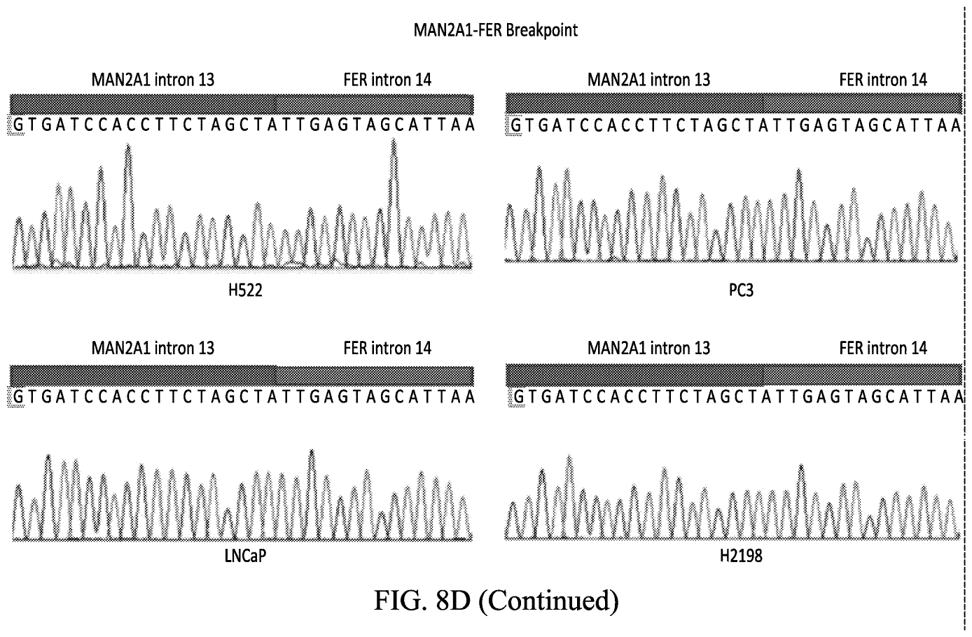

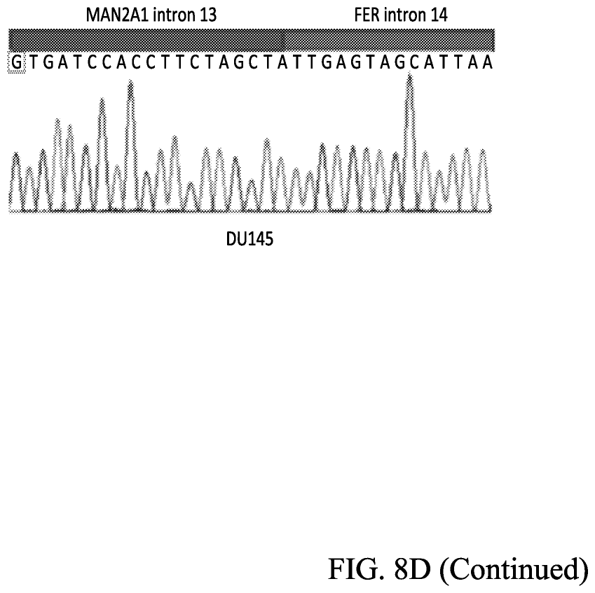

Expression of MAN2A1-FER is frequent in human cancer cell lines. The generation of MAN2A1-FER gene fusion is the result of chromosome recombination in the 5q21-22 region. The expression of MAN2A1-FER was frequently detected in primary liver cancer and serum samples. To investigate the expression of MAN2A1-FER in liver cancer cell lines, 7 HCC cell lines were tested for MAN2A1-FER expression using TaqMan qRT-PCR. Six cell lines including HUH7, SNU387, SNU449, SNU475, SNU182, and HEPG2 were positive for MAN2A1-FER mRNA, while HEP3B was negative (FIG. 1A and FIG. 8A). To investigate whether other HCC cell lines contained the same breakpoint as HUH7, Taqman RT-PCR and Sanger's sequencing were performed on the genome DNA of SNU387, SNU449, SNU475, SNU182, and HEPG2 cells. The results indicated that all these cell lines positive for MAN2A1-FER had an identical breakpoint, suggesting a common mechanism of chromosome recombination that led to the generation of MAN2A1-FER gene fusion (FIG. 1B and FIG. 8B). Immunoblot analysis using the antibodies specific for the C-terminus of FER confirmed the fusion protein expression of MAN2A1-FER in all the HCC cell lines positive for the fusion genes while negative for HEP3B cells, HUH7ko cells where MAN2A1-FER expression was disrupted, and a normal liver sample.

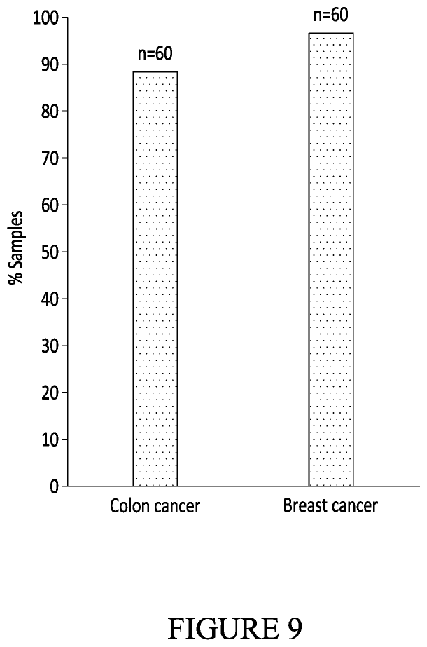

To investigate whether MAN2A1-FER gene fusion is widespread among human cancer cell lines, 3 prostate cancer (PC3, DU145, and LNCaP), 4 glioblastoma multiforme (98G, U118, U138, and LN229), 3 breast cancer (MCF7, MDA-MB231, and MDA-MB330), 4 non-small cell lung cancer (H358, H1299, H1298, and H522), 2 colon cancer (HCT8 and HCT15), and one renal cell carcinoma (293) cell lines were analyzed for MAN2A1-FER mRNA expression. The results showed that all these cancer cell lines were positive for MAN2A1-FER (FIG. 1A and FIG. 8A). All cancer cell lines positive for MAN2A1-FER mRNA shared the same breakpoint in the chromosome level (FIG. 1B and FIGS. 8B-8C). A distinct MAN2A1-FER chimera protein in the cell lines positive for MAN2A1-FER gene fusion was detected using the antibodies specific for the C-terminus of FER tyrosine kinase (FIG. 1C). High-frequency expressions of MAN1A1-FER were also detected in colon and breast cancers (FIG. 9). These results show that MAN2A1-FER was frequent in human malignancies.

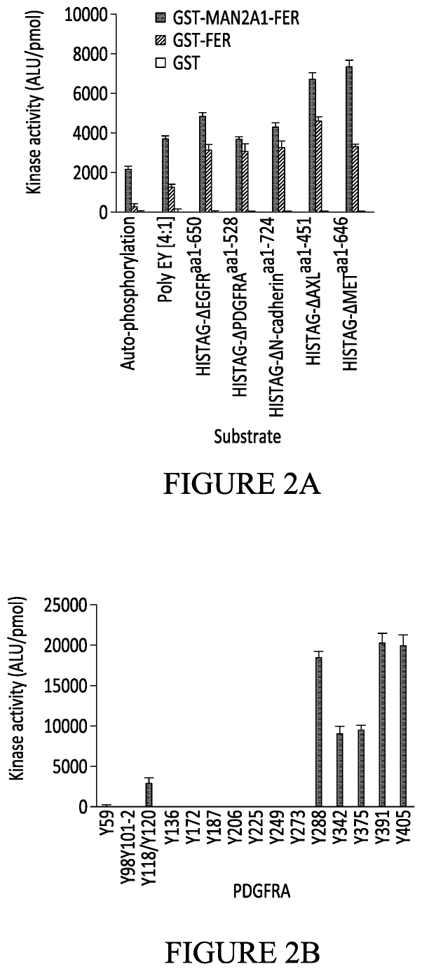

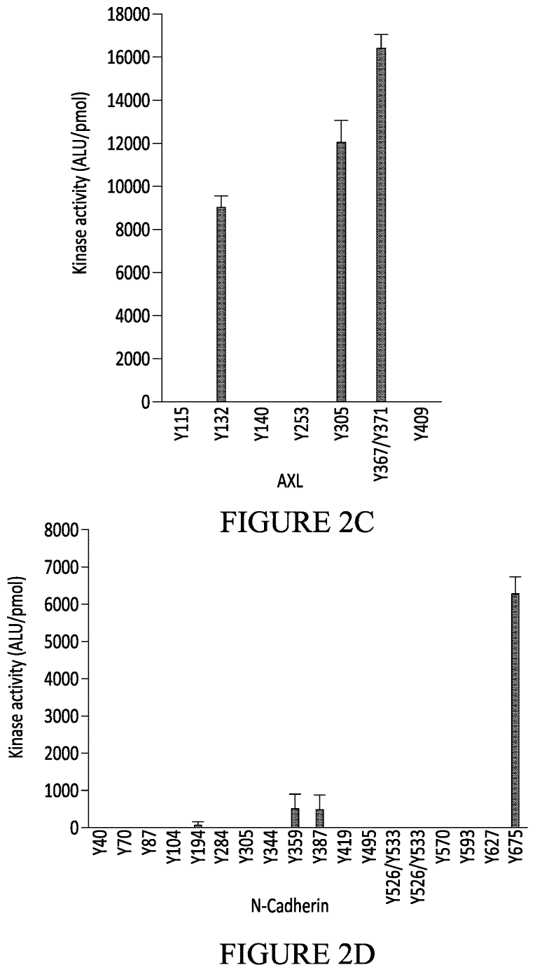

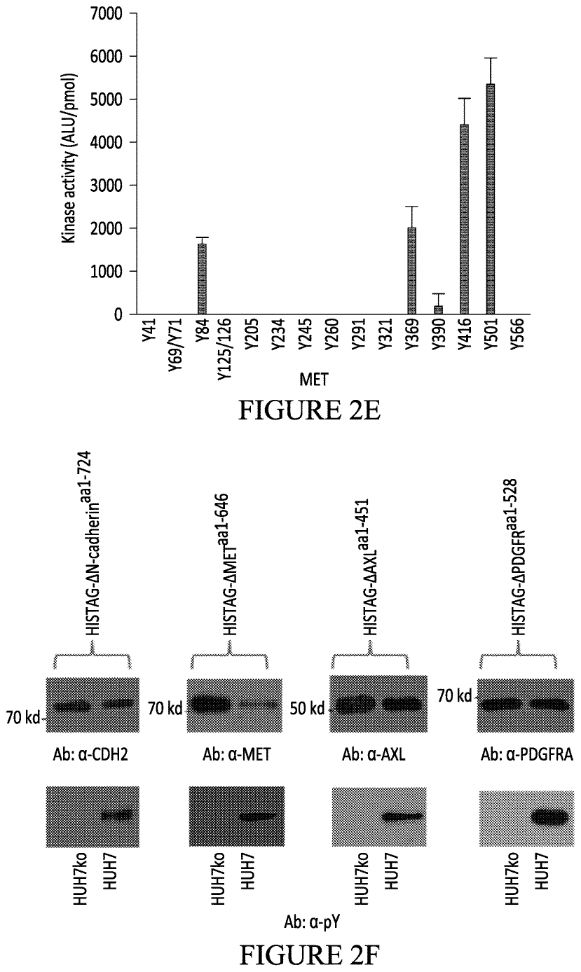

MAN2A1-FER kinase phosphorylated multiple transmembrane proteins in vitro and in vivo. MAN2A1-FER chimera protein is mostly localized in the Golgi apparatus in an orientation that can contact the extracellular domains of multiple transmembrane proteins. To investigate whether MAN2A1-FER induced the phosphorylation of extracellular domains of transmembrane proteins, the extracellular domains of PDGFRA (aa1-528), MET (aa1-646), AXL (aa1-451), and N-cadherin (aa1-723) were ligated into pET28a(+) vector to produce HisTAG fusion proteins in E. coli. Kinase analysis indicated that both GST-FER and GST-MAN2A1-FER induced the phosphorylation of these truncated proteins, similar to the results obtained from synthetic substrate poly E:Y (4:1) or the N-terminus of EGFR (FIG. 2A). The N-terminus of PDGFRA contained 17 tyrosine residues. To examine which tyrosine residue was phosphorylated, a series of 24-25 amino acid peptides corresponding to the regions containing tyrosine residues in the extracellular domain of PDGFRA were applied to GST-MAN2A1-FER tyrosine kinase assay. The results showed that significant phosphorylation occurred at 7 tyrosine residues (Y180/Y120, Y288, Y342, Y375, Y391, and Y405, FIG. 2B). For the extracellular domain of AXL, significant phosphorylation was identified at 4 positions (Y132, Y305, and Y367/Y371, FIG. 2C), while for N-cadherin (FIG. 2D) and MET (FIG. 2E), the phosphorylation occurred at 3 (Y359, Y387, and Y675) and 5 (Y84, Y369, Y390, Y416, and Y501) positions, respectively. To examine whether the ectopic phosphorylation of PDGFRA N-terminus occurred in vivo, HisTAG-ΔPDGFR^{aa1-528} was constructed into pCMV13 vector to express as a truncated PDGFRA protein in HUH7 where MAN2A1-FER was positive. As shown in FIG. 2F, the extracellular domain of PDGFRA was positive for tyrosine phosphorylation, as demonstrated by the recognition of the protein with an anti-phosphotyrosine antibody. In contrast, the immunoblotting became negative in HUH7ko cells, where MAN2A1-FER expression was disrupted. Similar findings were identified in the extracellular domains of AXL (HisTAG-ΔAXL^{aa1-451}), MET (HisTAG-ΔMET^{aa1-646}), and N-Cadherin (HisTAG-ΔN-Cadherin^{aa1-723}) These results suggest that the phosphorylation of extracellular domains of PDGFRA, AXL, MET, and N-cadherin were MAN2A1-FER dependent.

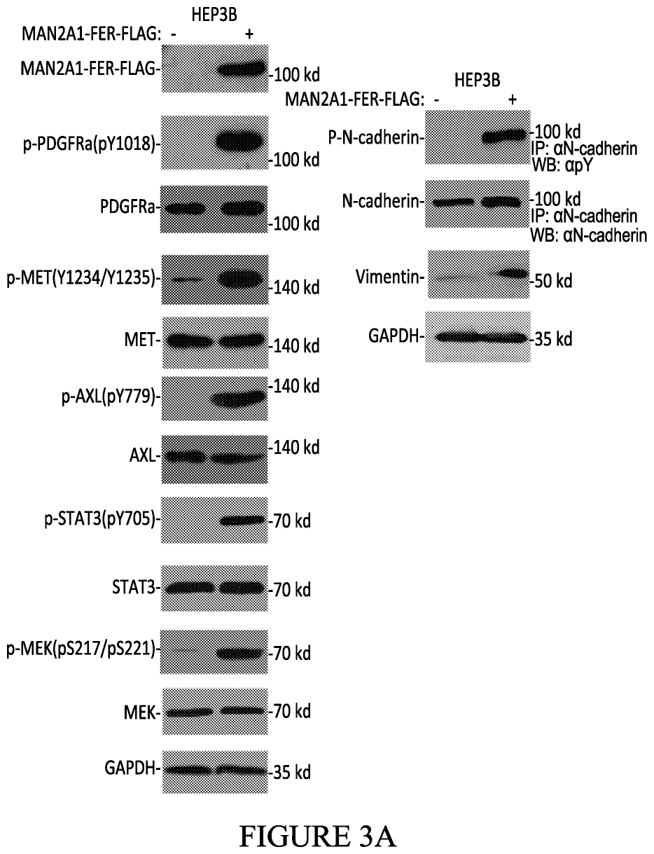

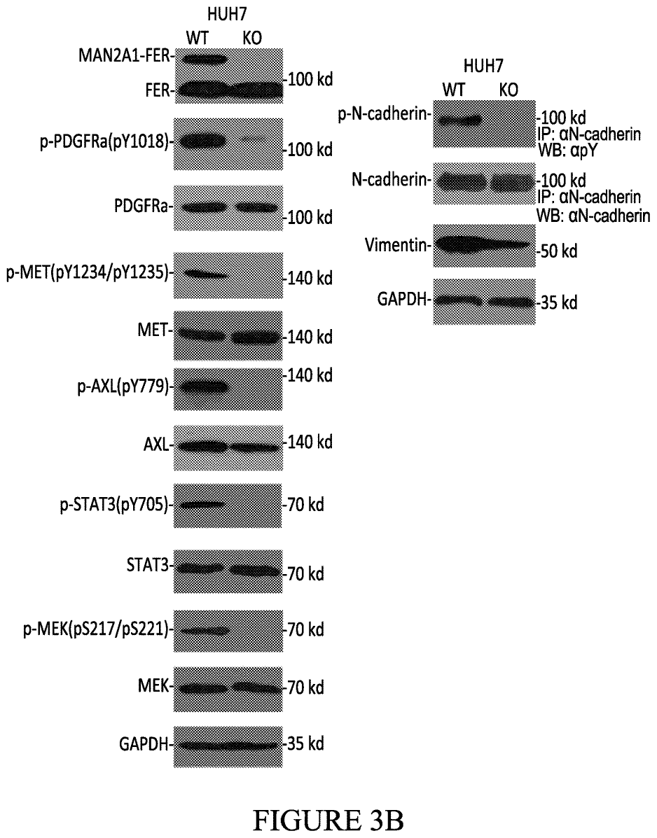

MAN2A1-FER activated signaling cascades of multiple transmembrane proteins. To investigate the impact of ectopic phosphorylation on PDGFRA, HEP3B cells where MAN2A1-FER was negative were transformed with pCDNA4-MAN2A1-FER-FLAG/pCDNA6TO. The induction of expression of MAN2A1-FER-FLAG by tetracycline showed a dramatic increase of phosphorylation of tyrosine 1018 in PDGFRA. This was accompanied by an increase of the phosphorylation of MEK and STAT3 (FIG. 3A). The expression of MAN2A1-FER-FLAG in HEP3B cells also increased the phosphorylation of tyrosine 1234/1235 of MET and tyrosine 779 of AXL and the activations of their common downstream signaling molecules of STAT3 and MEK. To examine whether the removal of MAN2A1-FER had the opposite effect on the activation of PDGFR, MET, and AXL, HUH7ko cells where MAN2A1-FER was disrupted were examined for the phosphorylation of Y1018 of PDGFRA. The results showed a significant decrease of PDGFRA activation based on Y1018 phosphorylation level (FIG. 3B). The downstream signaling molecules MEK and STAT3 also showed deactivation. The deactivation of MET and AXL signaling was also identified. The phosphorylation of N-cadherin by MAN2A1-FER-FLAG in HEP3B cells was accompanied by increased expression of vimentin (FIG. 3A), while disruption of MAN2A1-FER in HUH7ko cells reduced the expression of the same protein (FIG. 3B). These results show that MAN2A1-FER promotes multiple pro-growth and epithelial-mesenchyme transition in liver cancer cells.

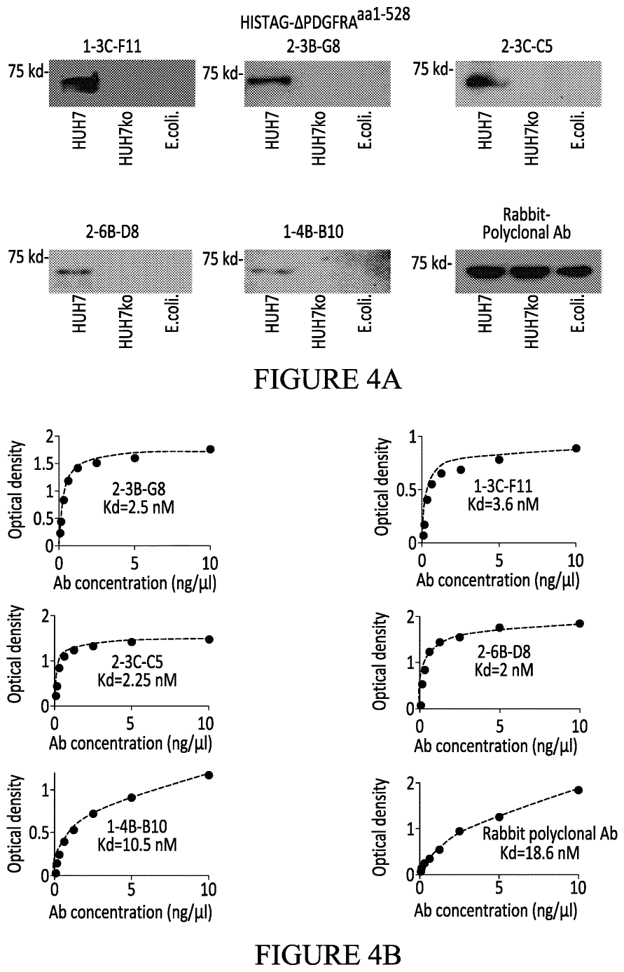

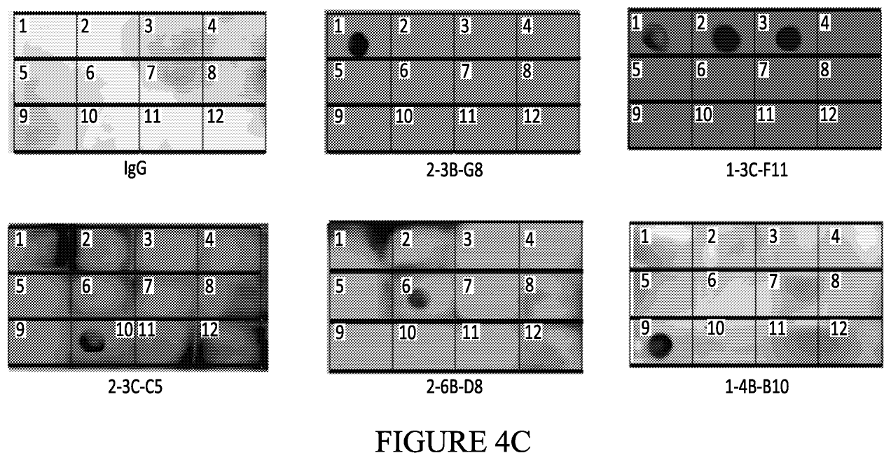

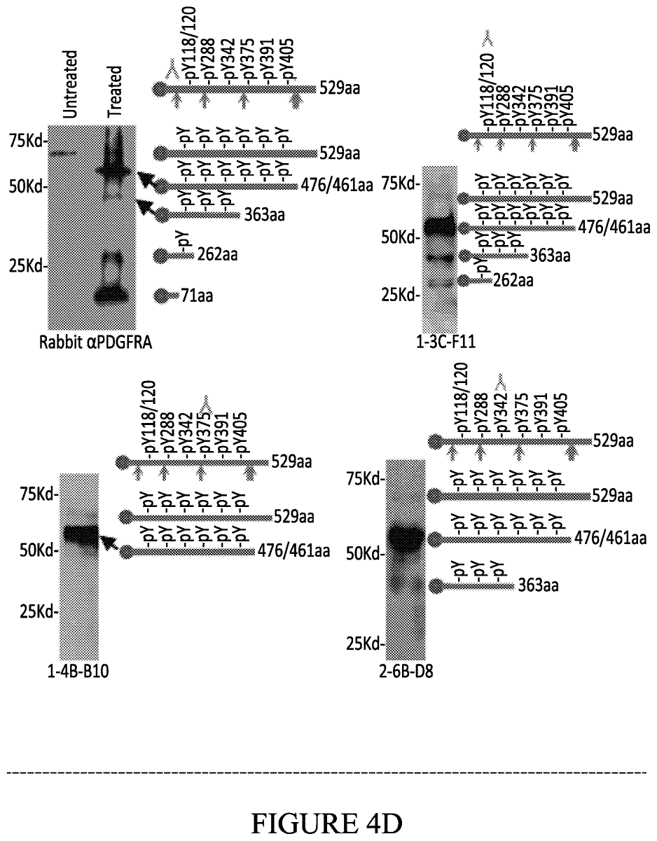

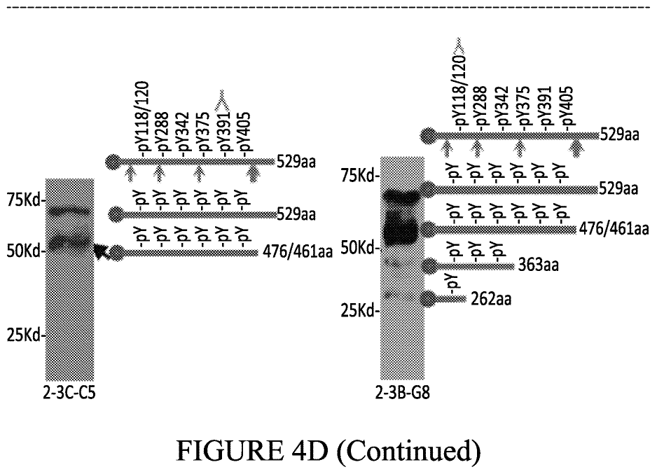

Mouse monoclonal antibody specific for ectopic phosphorylation of PDGFRA. The ectopic phosphorylation in the extracellular domains of growth factor receptors by MAN2A1-FER presents a targeting opportunity against these pathological phosphorylations. Next, the C57B16 mice were immunized with HisTAG-ΔPDGFRA^{aa1-528} (ectopically phosphorylated by MAN2A1-FER) expressed from HUH7 cells. After three rounds of immunization and antibody screening, the antisera positive for HisTAG-ΔPDGFRA^{aa1-528} of HUH7 from one of the five animals were obtained. Hybridoma cells were generated after fusing the spleen lymphocytes from the animal with Sp2/0-Ag14 cells. The hybridoma clones were screened for antibodies positive for HisTAG-ΔPDGFRA^{aa1-528} of HUH7 but negative for HisTAG-ΔPDGFRA^{aa1-528} of HUH7ko or HisTAG-PDGFRA^{aa1-528} of E. coli. As shown in FIG. 4A, five hybridoma clones were found to produce antibodies specific for the ectopically phosphorylated HisTAG-ΔPDGFRA^{aa1-528} from HUH7 cells but unreactive with HisTAG-ΔPDGFRA^{aa1-528} from HUH7ko or E. coli. To examine the binding affinity of these antibodies for the phosphorylated PDGFRA N-terminus, enzyme-linked immunosorbent assays were performed using the antibodies with different concentrations of the HisTAG-ΔPDGFRA^{aa1-528} from HUH7 cells. The results showed that these antibodies have dissociation constant (kd) values ranging from 2 to 10 nM (FIG. 4B), suggesting a high affinity of the antibodies with the antigen.

To identify which phosphotyrosine residue in the extracellular domain of PDGFRA these antibodies bound, a series of phospho-peptides corresponding 6 potential phosphotyrosine targets and their unphosphorylated counterparts were synthesized. As shown in FIG. 4C, the antibody from clone 2-3B-G8 appeared to bind to phospho-peptide (pY118/pY120) corresponding to aa106-130 of PDGFRA. Interestingly, the antibody did not bind the same peptides that contain only pY118 (well 2) or pY120 (well 3), nor the unphosphorylated peptide (well 12). On the other hand, the antibody from 1-3C-F11 clone bound to the same peptide containing pY118/pY120 (well 1), pY118 (well 2), or pY120 (well 3), but not the unphosphorylated control peptide (well 12). The antibody from clone 2-3C-C5 recognized the tyrosine phosphorylated (pY391, well 10) peptide corresponding to aa382-402 of PDGFRA but not the unphosphorylated counterpart (well 11, FIG. 4C). The antibody from clone 2-6B-D8 was specific for phosphotyrosine 342 (well 6) of PDGFRA, while the antibody from clone 1-4B-B10 was for phosphotyrosine 375 (well 9, FIG. 4C). To examine whether these antibodies also recognized the same epitopes in vivo, the HisTAG-ΔPDGFRA^{aa1-528} from HUH7 cells were partially digested with benzoic acid, which specifically cleaved at the tryptophan residue in a protein sequence. The partially digested HisTAG-ΔPDGFRA^{aa1-528} was purified through HisTAG column, resolved in 15% sodium dodecyl sulfate-polyacrylamide gel electrophoresis, and immunoblotted with each of these monoclonal antibodies. As shown in FIG. 4D, all the results in vivo were consistent with those in vitro.

TABLE 4
Tyrosine phosphorylated peptides and their unphosphorylated controls
corresponding to the extracellular domain of PDGFRA.

| Peptide | Position of Y | | |
|---|---|---|---|
| No. | phosphorylation | Peptide Sequence | SEQ ID NO |
| 1 | pY118/pY120 | QTEENELEGRHIpYIpYVPDPDVAFVP | SEQ ID NO.: |
| | | | 115 |
| 2 | pY118/pY120 | QTEENELEGRHIpYIYVPDPDVAFVP | SEQ ID NO.: |
| | | | 116 |
| 3 | pY120 | QTEENELEGRHIYIpYVPDPDVAFVP | SEQ ID NO.: |
| | | | 117 |
| 4 | pY288 | PEATVKDSGDpYECAARQATREVK | SEQ ID NO.: |
| | | | 118 |
| 5 | Y288 | PEATVKDSGDYECAARQATREVK | SEQ ID NO.: |
| | | | 119 |
| 6 | pY342 | VKHFVVEVRApYPPPRISWLKN | SEQ ID NO.: |
| | | | 120 |
| 7 | Y342 | VKHFVVEVRAYPPPRISWLKN | SEQ ID NO.: |
| | | | 121 |
| 8 | Y375 | TDVEKIQEIRYRSKLKLIRAK | SEQ ID NO.: |
| | | | 122 |
| 9 | pY375 | TDVEKIQEIRpYRSKLKLIRAK | SEQ ID NO.: |
| | | | 123 |
| 10 | pY391 | IRAKEEDSGHpYTIVAQNEDAV | SEQ ID NO.: |
| | | | 124 |
| 11 | Y391 | IRAKEEDSGHYTIVAQNEDAV | SEQ ID NO.: |
| | | | 125 |
| 12 | Y118/Y119 | QTEENELEGRHIYIYVPDPDVAFVP | SEQ ID NO.: |
| | | | 126 |

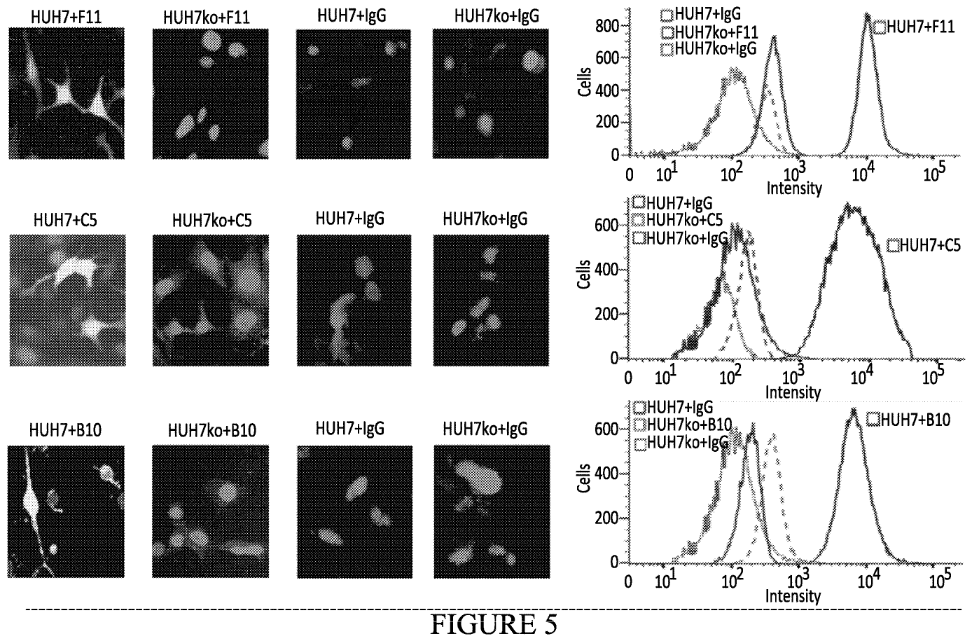

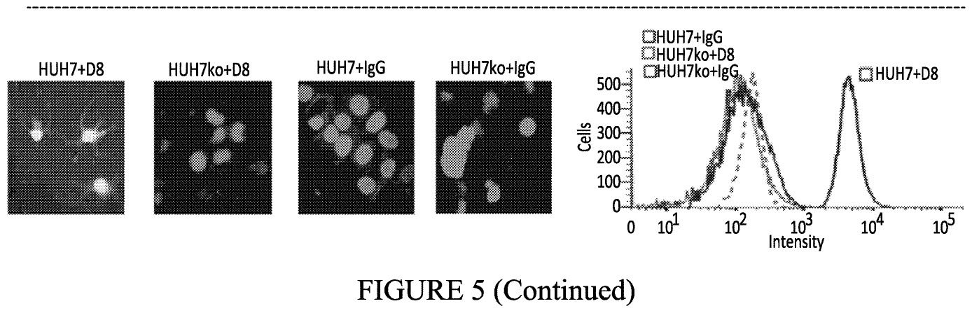

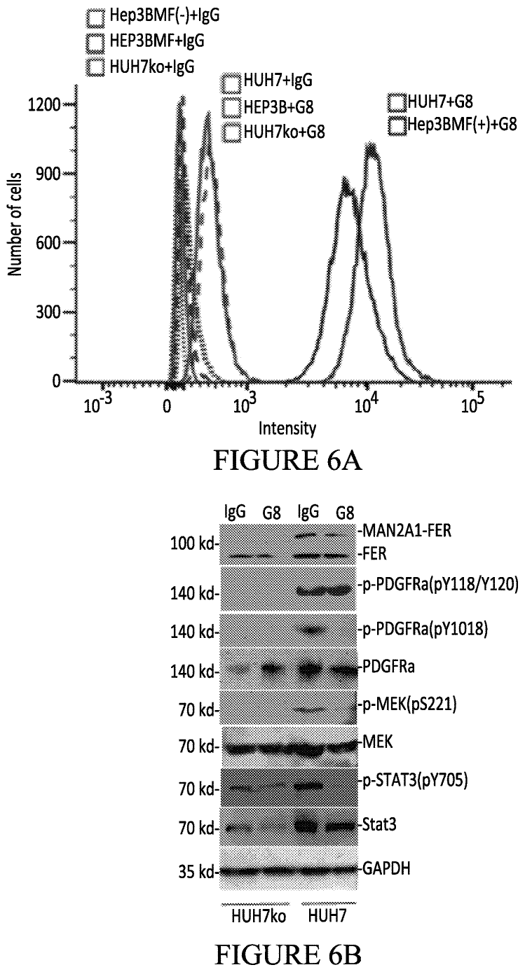

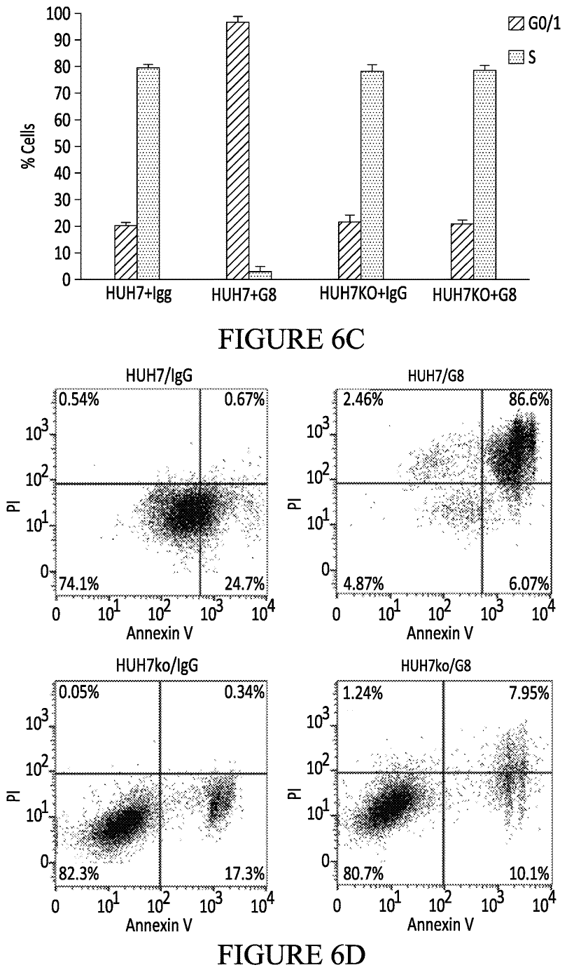

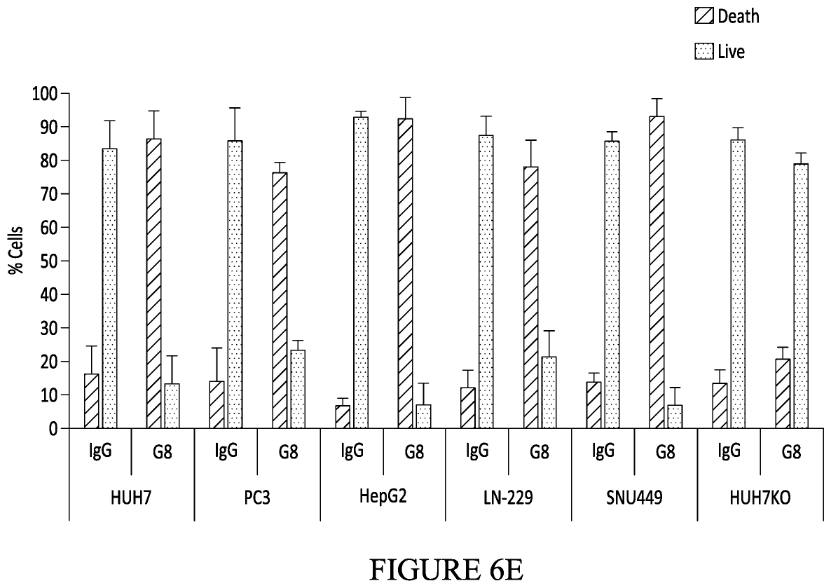

The binding of monoclonal antibodies specific for the ectopically phosphorylated extracellular domain of PDGFRA was MAN2A1-FER fusion dependent. To investigate whether the antibodies specific for the ectopically phosphorylated extracellular domain-bound PDGFRA epitopes in live liver cancer cells, unfixed HUH7 cells were immunostained with antibodies from 1-3C-F11, 2-3C-C5, 1-4B-B10, 2-6B-D8, and 2-3B-G8 (FIG. 5 and FIG. 6A). The results showed strong immunofluorescence staining of most HUH7 cells by all these antibodies. However, the immunostaining activity of these antibodies disappeared when the expression of MAN2A1-FER was disrupted in HUH7 cells, showing that the binding of these antibodies with HUH7 cells was dependent on the expression of MAN2A1-FER fusion protein.

Monoclonal antibody from hybridoma clone 2-3B-G8 induced growth arrest and cell death of HUH7 cells. Antibody 2-3B-G8 stained strongly on HUH7 cells or HEP3B cells transformed with MAN2A1-FER-FLAG (FIG. 6A) but was largely negative on HEP3B cells which were negative for MAN2A1-FER fusion or HUH7ko where MAN2A1-FER was disrupted in genome level. To investigate the impact of 2-3B-G8 antibody on signal transduction mediated by MAN2A1-FER, HUH7 cells were treated with 10 ng/ml 2-3B-G8 antibody for 6 hours. The results showed that treatment of 2-3B-G8 antibody significantly reduced the activation of PDGFRA, MEK, and STAT3 in HUH7 cells in comparison with the controls (FIG. 6B). The impact of 2-3B-G8 antibody on HUH7 cells are concordance with the presence of pY118/pY120 epitope in PDGFRA. In contrast, the impact of 2-3B-G8 antibody on MEK and STAT3 activation was minimal in HUH7ko cells where pY118/pY120 epitope was absent. Cell cycle analyses showed that the treatment of 2-3B-G8 antibody on HUH7 blocked cell entry into the S phase by 25.6 fold (3.1% vs. 79.7%, p<0.001) in comparison with the non-specific IgG controls (FIG. 6C). However, such dramatic impact disappeared when MAN2A1-FER was disrupted in HUH7 cell line, indicating that the growth inhibition by 2-3B-G8 antibody was entirely dependent on MAN2A1-FER expression. To examine the consequence of 2-3B-G8 treatment on cell lines that were positive for MAN2A1-FER, several human cancer cell lines positive for MAN2A1-FER, including HUH7, HEPG2, SNU449, LN-229, PC3 along with HUH7ko were examined for cell death after the treatment of 2-3B-G8 antibody. As shown in FIG. 6D, the treatment of 2-3B-G8 antibody on HUH7 cells induced ~3.7 fold increase of cell death (95.1% vs. 25.5%, p<0.01). However, this impact was largely eliminated in HUH7ko cells (19.3% vs. 17.7%, p=0.27). Similar large increases of cell death were found for other liver cancer cell lines such as HEPG2 (13.1 fold, p<0.01) and SNU449 (6.7 fold, <0.01), prostate cancer cell line PC3 (5.4 fold, p<0.01) and glioblastoma cell line LN-229 (7.6 fold, p<0.01), when treated with 2-3B-G8 antibody (FIG. 6E).

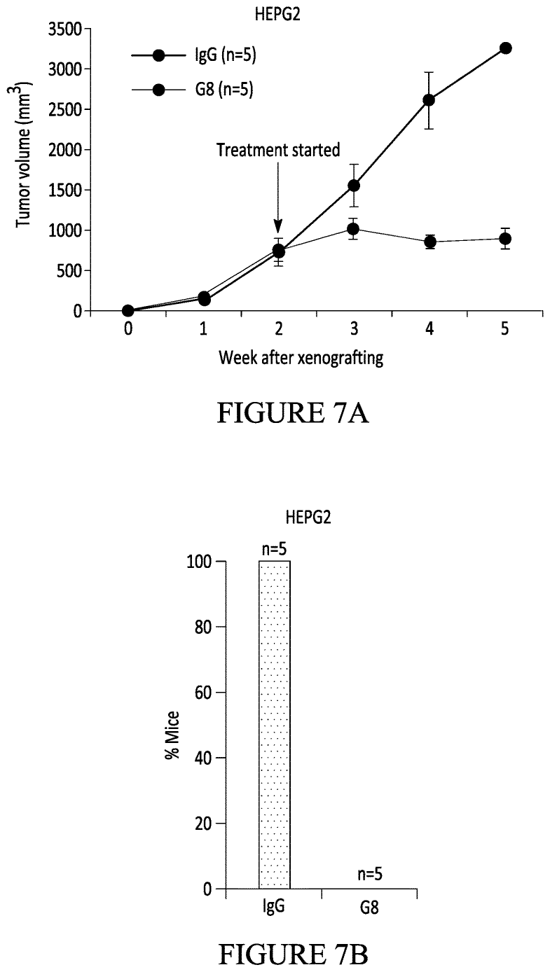

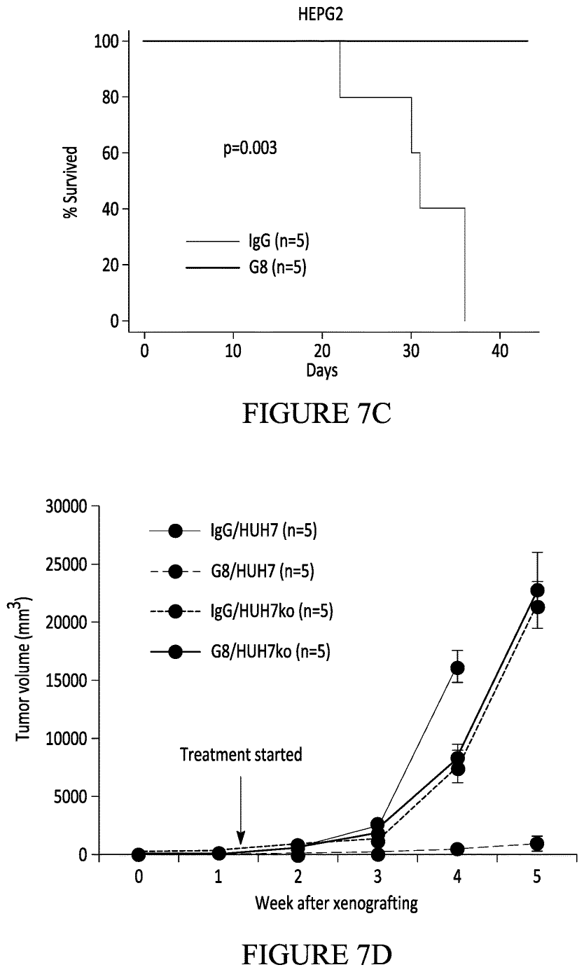

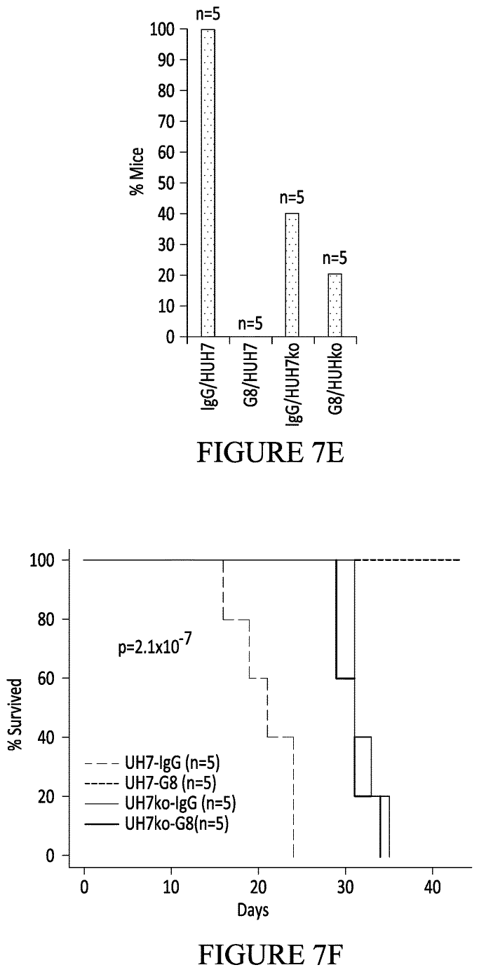

Monoclonal antibody from hybridoma clone 2-3B-G8 alleviated tumor burden, reduced metastases, and decreased mortality of xenografted liver cancer cell lines. To investigate whether 2-3B-G8 antibody had therapeutic value in treating cancer positive for MAN2A1-FER, liver cancer cell line HEPG2 was xenografted into the subcutaneous region of SCID mice. When HEPG2 tumor reached an average size of 752 mm³, the mice were treated with the MMAE conjugated 2-3B-G8 antibody through tail vein injection twice a week. As shown in FIG. 7A, the treatment of 2-3B-G8-MMAE blunted the increase of tumor volume, and produced a mild regression of the tumor size from its peak. In contrast, the IgG-MMAE-treated HEPG2 tumors continued their exponential growth. The 2-3B-G8-MMAE treated animals have no invasion or metastasis, while all the animals of the control group have some metastasis from the xenografted HEPG2 tumor (FIG. 7B). All the control-treated animals died within 5 weeks of xenografting, while all 2-3B-G8-MMAE-treated animals survived the same period (FIG. 7C). When SCID mice xenografted with HUH7 cells were treated with 2-3B-G8-MMAE, the growth of the tumors was also significantly slowed down (FIG. 7D). This contrasted with the exponential nature of tumor growth in the IgG-MMAE control group. However, the inhibition effect of tumor growth by 2-3B-G8-MMAE was largely eliminated after MAN2A1-FER was disrupted. The 2-3B-G8-MMAE treated animals had no metastasis, while all the control group mice had significant metastasis (FIG. 7E). On the other hand, such metastasis prevention by 2-3B-G8-MMAE was not significant in HUH7ko mice. All the HUH7 xenografted SCID mice treated by IgG-MMAE died 35 days after the tumor cell implantation, while all the mice treated by 2-3B-G8-MMAE survived the same period (FIG. 7F). The survival impact by 2-3B-G8-MMAE, however, was not found for HUH7ko cells xenografted animals.

### DISCUSSION

MAN2A1-FER is one of the frequent fusions in human HCC and is also widely present in other human malignancies. The previous report showed that MAN2A1-FER was translocated to the Golgi apparatus. Since the FER kinase domain is exposed in the lumens of the Golgi, it can have physical contact with the extracellular domains of a variety of transmembrane proteins due to the membrane protein glycosylation process brought by the MAN2A1 domain. In this study, MAN2A1-FER phosphorylated the extracellular domains of some critical growth factor receptors and EMT regulator and activated the pro-growth and transformation signaling. This is the first example of the activation of multiple signaling pathways through ectopic phosphorylation by a pathological chimera protein. Such multi-pathway activations have significant implications for liver cancer management and treatment. First, many of the previous observations on growth factor receptor activation in HCC are the results of MAN2A-FER gene fusion due to the high frequency of this fusion genes in the HCC samples. Second, single pathway blockade through small molecules is not sufficient to kill off HCC cancer cells that are positive of MAN2A1-FER because of multiple bypasses of the growth signaling.

PDGFR is one of the crucial growth factor receptors essential for the growth and invasion of liver cancers. At least 6 tyrosine residues in the extracellular domain of PDGFRA were phosphorylated by MAN2A1-FER. The phosphorylation of these tyrosine residues activated the kinase domain of PDGFRA. The phosphorylation of the extracellular domain makes the extracellular domain of PDGFRA more soluble and acidic. These chemical changes in the protein help to unfold the domain secondary structure. That, in turn, induces allosteric change and leads to dimerization of PDGFR and the activation of its kinase domain. Indeed, similar promotion of dimerization and activation of EGFR kinase by MAN2A1-FER through phosphorylation of EGFR tyrosine 88 in the extracellular domain was previously observed.

In cell level, the exposure of phosphotyrosine epitopes of PDGFRA on the cell surface make the cancer cells distinctive from the normal hepatocytes immunologically. Since these epitopes are the results of the enzyme activity of MAN2A1-FER kinase, a large number of pathological phosphotyrosine epitopes of PDGFRA are generated from a few copies of MAN2A1-FER protein. Many more similar phosphotyrosine epitopes are present in other membrane proteins in MAN2A1-FER positive cells because the mannosidase and glycoside hydrolase domains of the fusion protein bring many “to be glycosylated” membrane proteins to the proximity of the FER kinase. Thus, developing immunological interventions against these cancer antigens is an effective means to treat HCC. The five monoclonal antibodies developed in this study are first in their class of immune reagents against tumor-specific antigens that are generated by ectopic phosphorylation. Unlike small molecules for PDGFR, which do not discriminate PDGFR from normal versus cancer cells, these antibodies do not recognize PDGFRA in the benign cells and thus have no impact on benign tissues. As a result, the side-effect of these “drugs” are much less than those of small molecules. This study shows that the antibodies generated against these ectopic epitopes are effective in targeting cancers with high specificity. The immune intervention based on these antibodies lay a foundation for future diagnosis and treatment for liver cancers that are positive for MAN2A1-FER gene fusion, since these antibodies can be humanized to form diagnostic imaging reagents such as fluorine labeled antibody specific for MAN2A1-FER positive cancers, and to form therapeutic reagents such as drug-conjugated/radio-labeled antibodies, chimera antigen receptor T cells, or cancer cell targeting delivery vehicles in the cancer treatment.

### Example 2: Validation of Partially Humanized and Fully Humanized 2-3B-G8 Antibodies

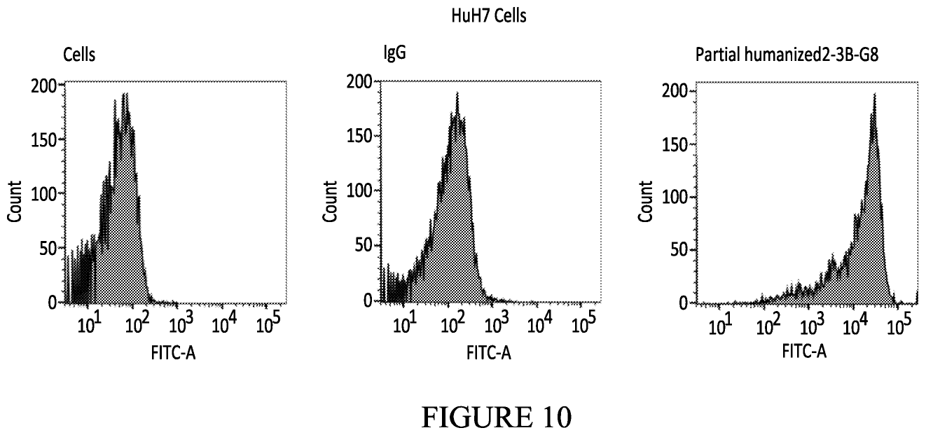

A partially humanized antibody was developed from clone 2-3B-G8. The heavy/light chain protein sequences of the partially humanized antibody correspond to SEQ ID NO.: 13/SEQ ID NO.: 12. Validation was performed in HUH7 cells. HUH7 cells were stained without primary antibody (FIG. 10, left), with non-specific IgG (FIG. 10, middle); or with the partially humanized 2-3B-G8 antibody (FIG. 10, right). Staining of HUH7 cells was greatly increased following contact of cells using the partially humanized 2-3B-G8 antibody in comparison to the cells contacted without primary antibody or with a non-specific primary antibody.

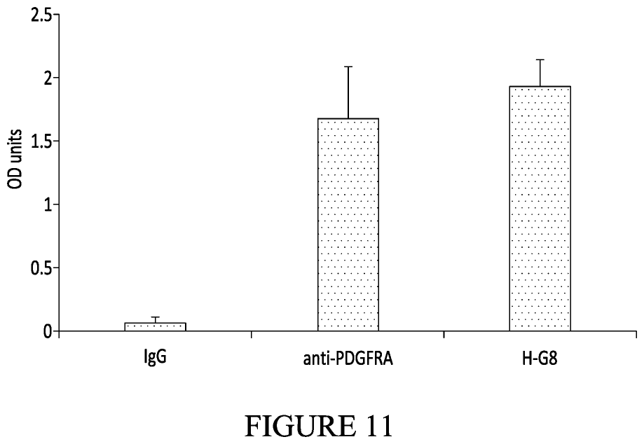

A fully humanized antibody was developed from clone 2-3B-G8. The heavy/light chain protein sequences of the fully humanized antibody correspond to SEQ ID NO.: 13/SEQ ID NO.: 14. Validation was performed by ELISA using phospho-HisTAG-PDGFRA^{aa1-528} Optical densities were low following exposure to non-specific IgG antibody. Optical densities were increased following exposure to antibody specific for the N-terminus of human PDGFRA (anti-PDGFRA) or the fully humanized 2-3B-G8 antibody (FIG. 11).

### Example 3: Evaluation of Antibody-Directed Lipid Nanoparticles

The efficacy of antibody-directed lipid nanoparticles (LNP) are tested using human HCC tumor xenografts in immune-deficient mice (SCID mice). HEPG2 (moderately aggressive) and HUH7 (very aggressive) human HCC cell lines are implanted in mice. Mice are administered humanized 2-3B-G8 antibody specific for pY118/pY120 of PDGFRA or 2-3B-G8-directed LNP. In control experiments, mice are administered human IgG or IgG-directed LNP. To evaluate specificity, mice are implanted with HUH7MFko cells, which have disrupted MAN2A1-FER, and treated with humanized 2-3B-G8, or 2-3B-G8-directed LNP. The LNP comprise a luciferase expression vector payload. In another experiment, the LNP comprise a vector encoding iCasp9/AD1903, which provides expression of caspase-9 following induction with AD1903. In another experiment, the LNP comprise doxorubicin as payload. In another experiment, the LNP comprise monomethyl auristatin E (MMAE) as payload.

In follow-up experiments, immune-competent C57B1 mice are implanted with HEPA1-6 cells transfected with pCDNA4-MAN2A1-FER-FLAG and pCMV-PDGFRA (called Hepa-MF-hPDGFRA cells). The mice are administered humanized 2-3B-G8 antibody specific for pY118/pY120 of PDGFRA, 2-3B-G8-directed LNP, human IgG, or IgG-directed LNP. In another experiment, the LNP comprise spCas9-EGFP vector, which encodes Streptococcus pyogenes Cas9 fused with eGFP, and gRNA specific for STAT3a, PDL1, and/or PDL2. The expression of STAT3, PDL1 and PDL2, are examined through immunoblotting. In another experiment, mice are administered 1) 2-3B-G8-directed LNP comprising spCas9-EGFP vector and gRNA specific for STAT3a, PDL1, and/or PDL2 and 2) 2-3B-G8-directed LNP comprising iCasp9/AD1903 vector. In another experiment, mice are administered 1) 2-3B-G8-directed LNP comprising spCas9-EGFP vector and gRNA specific for STAT3a, PDL1, and/or PDL2 and 2) 2-3B-G8-directed LNP comprising DM1 payload. In another experiment, mice are administered 1) 2-3B-G8-directed LNP comprising DM1 payload or 2) 2-3B-G8-directed LNP comprising iCasp9/AD1903 vector.

In follow-up experiments, the efficacy of 2-3B-G8-directed LNP for human HCC cell lines are tested in vitro. HEPG2, HUH7, and HUH7MFko cells are treated with humanized 2-3B-G8 antibody or 2-3B-G8-directed LNP. In control experiments, mice are administered human IgG or IgG-directed LNP. The LNP comprise an eGFP expression vector payload. In another experiment, the cells are treated with 2-3B-G8-directed LNP comprising iCasp9/AD1903 vector. In another experiment, the LNP comprise a vector encoding iCasp9/AD1903, which provides expression of caspase-9 following induction with AD1903. In another experiment, the LNP comprise doxorubicin as payload. In another experiment, the LNP comprise monomethyl auristatin E (MMAE) as payload. In another experiment, the LNP comprise spCas9-EGFP vector and gRNA specific for STAT3a, PDL1, and/or PDL2. The expression of STAT3, PDL1 and PDL2, are examined through immunoblotting. Flow cytometry are performed to assess 2-3B-G8 guided LNP delivery efficacy. Cell death are assessed through annexin V and propidium iodide flow cytometry. These experiments demonstrate that antibody-directed nanoparticles, e.g., 2-3B-G8-directed LNP, enhance the delivery of nanoparticles to HCC expressing MAN2A1-FER in vitro and in vivo. The antibody-directed nanoparticles additionally enhance the delivery and effectiveness of therapeutics for HCC expressing MAN2A1-FER.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and compositions of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps. Various patents, patent applications, publications, product descriptions, protocols, and sequence accession numbers are cited throughout this application, this present disclosures of which are incorporated herein by reference in their entireties for all purposes.

What is claimed is:

1. An antibody or antigen-binding fragment, wherein the antibody or antigen-binding fragment specifically binds to at least one phosphotyrosine in the extracellular domain of PDGFRA, wherein the light chain variable domain sequence comprises an amino acid sequence that is at least 95% identical to an amino acid sequence selected from the group consisting of SEQ ID NO.: 4, SEQ ID NO.: 15, SEQ ID NO.: 22, and SEQ ID NO.: 32.

2. The antibody or antigen-binding fragment of claim 1, wherein the heavy/light chain protein sequence comprises an amino acid sequence selected from the group consisting of SEQ ID NO.: 6/SEQ ID NO.: 2, SEQ ID NO.: 13/SEQ ID NO.: 12, SEQ ID NO.: 13/SEQ ID NO.: 14, SEQ ID NO.: 24/SEQ ID NO.: 20, SEQ ID NO.: 24/SEQ ID NO.: 28, SEQ ID NO.: 34/SEQ ID NO.: 30, and SEQ ID NO.: 34/SEQ ID NO.: 38.

3. The antibody or antigen-binding fragment of claim 1, wherein the heavy/light chain variable domain sequence comprises an amino acid sequence selected from the group consisting of SEQ ID NO.: 8/SEQ ID NO.: 4 and SEQ ID NO.: 8/SEQ ID NO.: 15.

4. The antibody or antigen-binding fragment of claim 1, wherein the light chain protein sequence comprises an amino acid sequence selected from the group consisting of SEQ ID NO.: 2, SEQ ID NO.: 12, SEQ ID NO.: 14, SEQ ID NO.: 20, SEQ ID NO.: 28, SEQ ID NO.: 30, and SEQ ID NO.: 38.

5. The antibody or antigen-binding fragment of claim 1, wherein the light chain variable domain sequence comprises an amino acid sequence selected from the group consisting of SEQ ID NO.: 4, SEQ ID NO.: 15, SEQ ID NO.: 22, and SEQ ID NO.: 32.

6. An antibody or antigen-binding fragment, wherein the antibody or antigen-binding fragment specifically binds to at least one phosphotyrosine in the extracellular domain of PDGFRA, wherein the antibody or antigen-binding fragment comprises a light chain CDR-1 (CDRL1), light chain CDR-2 (CDRL2), and light chain CDR-3 (CDRL3), wherein:
a) the CDRL1, CDRL2, and CDRL3 comprise amino acid sequences that are at least 95% identical to SEQ ID NO.: 9, SEQ ID NO.: 10, and SEQ ID NO.: 11, respectively;
b) the CDRL1, CDRL2, and CDRL3 comprise amino acid sequences that are at least 95% identical to SEQ ID NO.: 16, SEQ ID NO.: 17, and SEQ ID NO.: 18;
c) the CDRL1, CDRL2, and CDRL3 comprise amino acid sequences that are at least 95% identical to SEQ ID NO.: 25, SEQ ID NO.: 26, and SEQ ID NO.: 27; or
d) the CDRL1, CDRL2, and CDRL3 comprise amino acid sequences that are at least 95% identical to SEQ ID NO.: 35, SEQ ID NO.: 36, and SEQ ID NO.: 37.

7. The antibody or antigen-binding fragment of claim 6, wherein:
a) the CDRL1, CDRL2, and CDRL3 comprise amino acid sequences that are identical to SEQ ID NO.: 9, SEQ ID NO.: 10, and SEQ ID NO.: 11;
b) the CDRL1, CDRL2, and CDRL3 comprise amino acid sequences that are identical to SEQ ID NO.: 16, SEQ ID NO.: 17, and SEQ ID NO.: 18;
c) the CDRL1, CDRL2, and CDRL3 comprise amino acid sequences that are identical to SEQ ID NO.: 25, SEQ ID NO.: 26, and SEQ ID NO.: 27; or
d) the CDRL1, CDRL2, and CDRL3 comprise amino acid sequences that are identical to SEQ ID NO.: 35, SEQ ID NO.: 36, and SEQ ID NO.: 37.

8. The antibody or antigen-binding fragment of claim 1, wherein the antibody or antigen-binding fragment comprises a heavy chain variable domain sequence that is truncated to remove one or more heavy chain complementarity determining regions.

9. The antibody or antigen-binding fragment of claim 1, wherein the antibody or antigen-binding fragment comprises a heavy chain variable domain sequence that is truncated to remove all three heavy chain complementarity determining regions.

10. The antibody or antigen-binding fragment of claim 1, wherein the antibody or antigen-binding fragment comprises one, two, or three heavy chain complementarity determining regions.

11. The antibody or antigen-binding fragment of claim 1, wherein the antibody or antigen-binding fragment does not have heavy chain complementarity determining regions.

12. The antibody or antigen-binding fragment of claim 1, wherein the antibody or antigen-binding fragment specifically binds to at least one phosphotyrosine selected from the list consisting of pY118/pY120, pY118, pY120, pY342, pY375, and pY391.

13. The antibody or antigen-binding fragment of claim 12, wherein the antibody or antigen-binding fragment specifically binds to pY118/pY120.

14. The antibody or antigen-binding fragment of claim 13, wherein the antibody or antigen-binding fragment does not bind to pY118, pY120, pY342, pY375, or pY391.

15. The antibody or antigen-binding fragment of claim 12, wherein the antibody or antigen-binding fragment does not bind to unphosphorylated extracellular domain of PDGFRA.

16. The antibody or antigen-binding fragment of claim 1, wherein the antigen-binding fragment is a Fv, Fab, Fab′, or F(ab′)₂.

17. The antibody or antigen-binding fragment of claim 1, wherein the antibody or antigen-binding fragment is conjugated to an anti-cancer agent.

18. A method of treating cancer, comprising administering a therapeutically effective amount of the antibody or antigen-binding fragment of claim 1, to a subject in need thereof.

19. The method of claim 18, wherein the cancer expresses MAN2A1-FER.

20. The method of claim 18, wherein the cancer is selected from the group consisting of: liver cancer, prostate cancer, brain cancer, glioblastoma multiforme, breast cancer, lung cancer, non-small cell lung cancer, colon cancer, and renal cell carcinoma.
